(19) United States
(12) Patent Application Publication
(10) Pub. No.: US 2014/0377857 A1
(43) Pub. Date: Dec. 25, 2014

(54) INTEGRATED ELECTRO-BIOREACTOR

(71) Applicant: The Regents of the University of California, Oakland, CA (US)

(72) Inventors: James C. Liao, Los Angeles, CA (US); Han Li, Qingdao (CN)

(21) Appl. No.: 14/375,782

(22) PCT Filed: Feb. 15, 2013
(86) PCT No.: PCT/US13/26518

§ 371 Date: Jul. 30, 2014

Related U.S. Application Data
(60) Provisional application No. 61/599,368, filed on Feb. 15, 2012.

(51) Int. Cl. C12M 1/00

(52) U.S. Cl. CPC C12M 21/12; C12M 29/04
USPC 435/297.1

(57) ABSTRACT

The disclosure provides a process and bioreactor that converts CO₂ to higher alcohols (e.g. isobutanol) using electricity as the energy source. This process stores electricity (e.g. from solar energy, nuclear energy, and the like) in liquid fuels that can be used as high octane number gasoline substitutes. Instead of deriving reducing power from photosynthesis, this process derives reducing power from electrically generated mediators, either H₂ or formate. H₂ can be derived from electrolysis of water. Formate can be generated by electrochemical reduction of CO₂. After delivering the reducing power in the cell, formate becomes CO₂ and recycles back. Therefore, the biological CO₂ fixation process can occur in the dark.

### CROSS REFERENCE TO RELATED APPLICATIONS

This application claims priority under 35 U.S.C. §119 to U.S. Provisional Application No. 61/599,368, filed Feb. 15, 2012, the disclosure of which is incorporated herein by reference.

### STATEMENT REGARDING FEDERAL SPONSORSHIP

This invention was made with Government support under Grant No. DE-AR0000085, awarded by the U.S. Department of Energy, Advanced Research Projects Agency. The Government has certain rights in this invention.

### BACKGROUND

Biofuels are an alternative for fossil fuels. For example, isobutanol can be used as a high octane fuel for four-stroke internal combustion engines, as a pure component or in any portion as a mixture with gasoline. It has a high energy density (36 MJ/Kg) and low heat of vaporization (0.43 MJ/Kg), both of which satisfy the requirements (energy density ≧32 MJ/Kg, heat of vaporization <0.5 MJ/Kg) specified by this FOA. The research octane number of isobutanol is 110, which also satisfies the requirement (>85).

### SUMMARY

The disclosure provides a bioreactor useful for the production of biofuels. The disclosure provides an integrated electro-bioreactor that allows simultaneous electrolysis and fermentation in the same tank. Electrolysis can be used to deliver a reducing mediator to a cell; the cell can use the reducing mediator to conduct various reactions, including CO₂ fixation or other redox reactions. However, electrolysis typically generates free radicals, which are toxic to the cells. As such direct integration of electrolysis unit with fermentation is difficult. The disclosure provides method and devices to isolate the anode such that the free radicals can be quenched before reaching the cell. This device allows the simultaneous electrolysis and bioreactor to proceed in the same tank. With this electro-bioreactor, electricity can be directly used to reduce chemicals that can diffuse into the cell to drive reduction of various compounds. One notable application is the electricity-driven reduction of CO₂, as described in further detail below.

The disclosure provides recombinant microorganisms that take advantage of the biological C—C bond formation capability without relying on inefficient photo energy conversion. Instead, reducing power is generated from electricity (including sunlight) to drive the metabolic process that forms C—C bonds necessary for liquid fuel synthesis. Thus, the microorganism of the disclosure utilizes man-made photo conversion and the biological C—C bond synthesis to make liquid fuels. The pathways engineered into microorganisms as described herein utilize electrically generated reducing mediators (H₂ or formate) to drive the “dark reaction” of CO₂ fixation. Both H₂ and formate can be used to reduce NAD(P)+ to NAD(P)H, which is then used as the reducing equivalent in CO₂ reduction, fuel synthesis, and ATP synthesis. Once CO₂ is fixed in a metabolic intermediate, such as pyruvate, it can be diverted to make isobutanol and other biofuels. The biological processes (H₂ or formate utilization, CO₂ fixation, fuel synthesis) can be independently or all engineered into the same cell so long as the pathway comprises CO₂ fixation and utilizes reducing mediators along with the specific biofuel pathway. Furthermore, bioreactors and electrolysis units can be integrated to form an electro-bio reaction unit.

The disclosure provides a recombinant microorganism capable of using H₂ or formate for reduction of CO₂ and wherein the microorganism produces an alcohol selected from the group consisting of 1-propanol, isobutanol, 1-butanol, 2-methyl 1-butanol, 3-methyl 1-butanol and 2-phenylethanol from CO₂ as the carbon source, wherein the alcohol is produced from a metabolite comprising a 2-keto acid. In one embodiment, the microorganism has a naturally occurring H₂ and/or formate reduction pathway and at least one recombinant enzyme for the production of an intermediate in the synthesis of the alcohol. In another embodiment, the microorganism comprises expression of a heterologous or overexpression of an endogenous carbon-fixation enzyme and heterologous or overexpression of a hydrogenase and/or formate dehydrogenase such that the microorganism can utilize H₂ and/or formate as a reducing metabolite. In any of the foregoing embodiments, the alcohol can be isobutanol. In yet another embodiment, the recombinant microorganism is obtained from a Ralstonia sp. parental organism. In another embodiment, the 2-keto acid is selected from the group consisting of 2-ketobutyrate, 2-ketoisovalerate, 2-ketovalerate, 2-keto 3-methylvalerate, 2-keto 4-methyl-pentanoate, and phenylpyruvate. In one embodiment, the microorganism comprises elevated expression or activity of a 2-keto-acid decarboxylase and an alcohol dehydrogenase, as compared to a parental microorganism. In one embodiment, the 2-keto-acid decarboxylase is selected from the group consisting of Pdc6, Aro10, Thi3, Kivd, and Pdc, or homolog thereof. In yet another embodiment, the 2-keto-acid decarboxylase is encoded by a nucleic acid sequence derived from a gene selected from the group consisting of PDC6, ARO10, THI3, kivd, and pdc, or homolog thereof. In a specific embodiment, the 2-keto-acid decarboxylase is encoded by a nucleic acid sequence derived from the kivd gene, or homolog thereof. In one embodiment, the alcohol dehydrogenase is Adh2, or homolog thereof. In another embodiment, the alcohol dehydrogenase is encoded by a nucleic acid sequence derived from the ADH2 gene, or homolog thereof. In another embodiment, the microorganism is selected from a genus of Escherichia, Corynebacterium, Lactobacillus, Lactococcus, Salmonella, Enterobacter, Enterococcus, Erwinia, Pantoea, Morganella, Pectobacterium, Proteus, Ralstonia, Serratia, Shigella, Klebsiella, Citrobacter, Saccharomyces, Dekkera, Klyveromyces, and Pichia. In one embodiment, not only does the organism comprise a pathway for utilizing H₂ or formate but the organism also has a modification in the biosynthetic pathway for the production of an amino acid to produce the alcohol. The microorganism can also have reduced ethanol production capability compared to a parental microorganism. For examples, the microorganism comprises a reduction or inhibition in the conversion of acetyl-coA to ethanol. The microorganism can comprise a reduction of an ethanol dehydrogenase thereby providing a reduced ethanol production capability. In specific embodiments of any of the foregoing the microorganism produces greater than 100 mg/L of isobutanol in 40 hours from sugar. In other specific embodiments of any of the foregoing, the microorganism produces greater than 150 mg/L of 3-methyl-1-butanol in 40 hours from sugar. In another embodiment, the microorganism produces 120 mg/L of isobutanol or 180 mg/L of 3-methyl-1-butanol. In one embodiment, the mircroorganis comprising a knockout of a gene encoding an enzyme for the production of PHB.

The disclosure provides an integrated bioreactor comprising (a) an anode; (b) a cathode; (c) a container comprising at least one wall and having at least one opening, wherein the anode and cathode are disposed within the container; (d) a liquid permeable separator, wherein the separator surrounds the anode defining an anode space, wherein the separator substantially confines free-radicals produced at the anode within the anode space; (e) at least one fluid inlet extending through the opening of the container into the container. In one embodiment, the at least one fluid inlet comprises at least 2 inlets. In yet another embodiment, the at least one fluid inlet is fluidly connected to a CO₂ sparger. In one embodiment, the separator comprises porous ceramic. In yet another embodiment, the bioreactor further comprises an aqueous media suitable for growth of a microorganism. In yet a further embodiment of any of the foregoing, the bioreactor further comprises a recombinant microorganism comprising: (i) a formate dehydrogenase capable of oxidizing formate and producing NADH or NADPH; and (ii) a heterologous enzyme selected from a ketoacid decarboxylase, an NADPH dependent aldehyde/alcohol dehydrogenase and a combination thereof, wherein the recombinant microorganism produces an alcohol selected from the group consisting of isobutanol, 1-butanol, 1-propanol, 2-methyl-1-butanol, 3-methyl-1-butanol and 2-phenylethanol from a 2-keto acid intermediate. In one embodiment, the formate dehydrogenase is heterologous. In another embodiment, the recombinant microorganism comprises a trans-hydrogenase. In yet another embodiment, the recombinant microorganism is a chemoautotrophic microorganism. In yet another embodiment, the recombinant microorganism is a lithoautotrophic microorganism. In yet another embodiment, the bioreactor further comprises a recombinant microorganism comprising: (i) a membrane and/or soluble hydrogenase capable of oxidizing formate and producing NADH or NADPH; and (ii) a heterologous enzyme selected from a ketoacid decarboxylase, an NADPH dependent aldehyde/alcohol dehydrogenase and a combination thereof, wherein the recombinant microorganism produces an alcohol selected from the group consisting of isobutanol, 1-butanol, 1-propanol, 2-methyl-1-butanol, 3-methyl-1-butanol and 2-phenylethanol from a 2-keto acid intermediate. In a further embodiment, the membrane and/or soluble hydrogenase is heterologous. In yet another embodiment, the recombinant microorganism comprises a trans-hydrogenase. In yet other embodiment, the recombinant microorganism is a chemoautotrophic microorganism. In yet another embodiment, the recombinant microorganism is a lithoautotrophic microorganism. In one embodiment, the microorganism comprises a carbon fixing enzyme. In a further embodiment, the carbon fixing enzyme is heterologous to the organism. In one embodiment, a biosynthetic pathway for the production of an amino acid in the organism is modified for production of the alcohol. In one embodiment, the 2-keto acid intermediate is selected from the group consisting of 2-ketobutyrate, 2-ketoisovalerate, 2-ketovalerate, 2-keto 3-methylvalerate, 2-keto 4-methyl-pentanoate, and phenylpyruvate. In another embodiment, the microorganism comprises reduced ethanol production capability compared to a parental microorganism. In yet another embodiment, the microorganism comprises a reduction or inhibition in the conversion of acetyl-coA to ethanol. In a further embodiment, the recombinant microorganism comprises a reduction of an ethanol dehydrogenase thereby providing a reduced ethanol production capability. In yet a further embodiment, the ethanol dehydrogenase is an adhE, homolog or variant thereof. In yet another embodiment, the microorganism comprises a deletion or knockout of an adhE, homolog or variant thereof. In another embodiment, the microorganism comprises expression or elevated expression of an enzyme in a biochemical pathway that converts pyruvate to alpha-keto-isovalerate. In one embodiment, the microorganism comprises elevated expression or activity of a 2-keto-acid decarboxylase and an alcohol dehydrogenase, as compared to a parental microorganism. In one embodiment, the 2-keto-acid decarboxylase is selected from the group consisting of Pdc, Pdc1, Pdc5, Pdc6, Aro10, Thi3, Kivd, and KdcA, a homolog or variant of any of the foregoing, and a polypeptide having at least 60% identity to any one of the foregoing and having 2-keto-acid decarboxylase activity. In another embodiment, the 2-keto-acid decarboxylase is encoded by a polynucleotide having at least 60% identity to a nucleic acid selected from the group consisting of pdc, pdc1, pdc5, pdc6, aro10, thi3, kivd, kdcA, a homolog or variant of any of the foregoing, or a fragment thereof and wherein the polynucleotide encodes a polypeptide having 2-keto acid decarboxylase activity. In yet another embodiment, the alcohol dehydrogenase is selected from the group consisting of Adh1, Adh2, Adh3, Adh4, Adh5, Adh6, Sfa1, a homolog or variant of any of the foregoing, and a polypeptide having at least 60% identity to any one of the foregoing and having alcohol dehydrogenase activity. In yet a further embodiment, the alcohol dehydrogenase is encoded by a polynucleotide having at least 60% identity to a nucleic acid selected from the group consisting of an adh1, adh2, adh3, adh4, adh5, adh6, sfa1 gene, and a homolog of any of the foregoing and wherein the polynucleotide encodes a protein having 2-alcohol dehydrogenase activity. In one embodiment, the recombinant microorganism comprises one or more deletions or knockouts in a gene encoding an enzyme that catalyzes the conversion of acetyl-coA to ethanol, catalyzes the conversion of pyruvate to lactate, catalyzes the conversion of fumarate to succinate, catalyzes the conversion of acetyl-coA and phosphate to coA and acetyl phosphate, catalyzes the conversion of acetyl-coA and formate to coA and pyruvate, condensation of the acetyl group of acetyl-CoA with 3-methyl-2-oxobutanoate (2-oxoisovalerate), isomerization between 2-isopropylmalate and 3-isopropylmalate, catalyzes the conversion of alpha-keto acid to branched chain amino acids, synthesis of Phe, Tyr, Asp or Leu, catalyzes the conversion of pyruvate to acetyl-coA, catalyzes the formation of branched chain amino acids, catalyzes the formation of alpha-ketobutyrate from threonine, catalyzes the first step in methionine biosynthesis, catalyzes the conversion of acetoacetyl-CoA to 3-hydroxy-butyryl-Coa, catalyzes the conversion of 3-hydroxy-butyryl-CoA to PHB, and catalyzes the catabolism of threonine. In another embodiment, the recombinant microorganism comprises one or more gene deletions selected from the group consisting of adhE, ldhA, frdBC, fnr, pta, pflB, leuA, leuB, leuC, leuD, ilvE, tyrB, poxB, ilvB, ilvI, ilvA, metA, tdh, phaA, phaB, phaC, homologs of any of the foregoing and naturally occurring variants of any of the foregoing. In yet still another embodiment, the microorganism comprises a genotype selected from the group consisting of: (a) a deletion or knockout selected from the group consisting of ΔadhE, ΔldhA, ΔfrdB, ΔfrdC, Δfnr, Δpta, ΔpflB, ΔleuA, ΔilvE, ΔpoxB, ΔilvA, ΔphaA, ΔphaB, ΔphaC and any combination thereof and comprising an expression or increased expression of kdc, ilvC, ilvD and adh2 wherein the microorganism produces isobutanol; and (b) a deletion or knockout selected from the group consisting of ΔadhE, ΔldhA, ΔfrdB, ΔfrdC, Δfnr, Δpta, ΔpflB, ΔilvE, ΔtyrB, ΔphaA, ΔphaB, ΔphaC and any combination thereof and comprising an expression or increased expression of kdc, LeuABCD, and adh2 wherein the microorganism produces 3-methyl 1-butanol. In one embodiment the microorganism has a naturally occurring H₂ and/or formate reduction pathway and at least one recombinant enzyme for the production of an intermediate in the synthesis of the alcohol. In another embodiment, the microorganism comprises expression of a heterologous or overexpression of an endogenous carbon-fixation enzyme and heterologous or overexpression of a hydrogenase and/or formate dehydrogenase such that the microorganism can utilize H₂ and/or formate as a reducing metabolite. In yet another embodiment, the microorganism comprises elevated expression or activity of: (a) an acetohydroxy acid synthase; (b) an acetohydroxy acid isomeroreductase; (c) a dihydroxy-acid dehydratase; (d) a 2-keto-acid decarboxylase; and (e) an alcohol dehydrogenase; as compared to a parental microorganism, and wherein the recombinant microorganism comprises at least one enzyme that can oxidize H₂ or formate to provide free electrons to reduce NAD to NADH or NADP to NADPH, and wherein the organism comprises a carbon fixing pathway that utilizes CO₂ as a carbon source and wherein the organism comprises at least one gene knockout or disruption encoding an enzyme selected from the group consisting of an ethanol dehydrogenase, a lactate dehydrogenase, a fumarate reductase, a phosphate acetyltransferase, a formate acetyltransferase, beta-ketothiolase (phaA), NADPH-linked acetoacetyl coenzyme A (acetyl-CoA) reductase (phaB), and PHB synthase (phaC) and any combination thereof, wherein the recombinant microorganism produces isobutanol. In another embodiment, the recombinant microorganism comprises elevated expression or activity of: (a) an acetolactate synthase; (b) an acetohydroxy acid isomeroreductase; (c) a dihydroxy-acid dehydratase; (d) a 2-keto-acid decarboxylase; and (e) an alcohol dehydrogenase; as compared to a parental microorganism, and wherein the recombinant microorganism comprises at least one enzyme that can oxidize H₂ or formate to provide free electrons to reduce NAD to NADH or NADP to NADPH, and wherein the organism comprises a carbon fixing pathway that utilizes CO₂ as a carbon source and wherein the organism comprises at least one gene knockout or disruption encoding an enzyme selected from the group consisting of an ethanol dehydrogenase, a lactate dehydrogenase, a fumarate reductase, a phosphate acetyltransferase, a formate acetyltransferase, beta-ketothiolase (phaA), NADPH-linked acetoacetyl coenzyme A (acetyl-CoA) reductase (phaB), and PHB synthase (phaC) and any combination thereof, wherein the recombinant microorganism produces isobutanol. In yet another embodiment, the microorganism comprises elevated expression or activity of: (a) acetohydroxy acid synthase or acetolactate synthase; (b) acetohydroxy acid isomeroreductase; (c) dihydroxy-acid dehydratase; (d) 2-isopropylmalate synthase; (e) isopropylmalate isomerase; (f) beta-isopropylmalate dehydrogenase; (g) 2-keto-acid decarboxylase; and (h) alcohol dehydrogenase; as compared to a parental microorganism, and wherein the recombinant microorganism comprises at least one enzyme that can oxidize H2 or formate to provide free electrons to reduce NAD to NADH or NADP to NADPH, and wherein the organism comprises a carbon fixing pathway that utilizes CO2 as a carbon source and wherein the organism comprises at least one gene knockout or disruption encoding an enzyme selected from the group consisting of an ethanol dehydrogenase, a lactate dehydrogenase, a fumarate reductase, a phosphate acetyltransferase, a formate acetyltransferase, beta-ketothiolase (phaA), NADPH-linked acetoacetyl coenzyme A (acetyl-CoA) reductase (phaB), and PHB synthase (phaC) and any combination thereof.

The disclosure provides a bioreactor for producing biofuels from a recombinant microorganism capable of using H₂ or formate for reduction of CO₂ the recombinant microorganism comprising a recombinant microorganism of the disclosure, the bioreactor comprising a porous divider that provides a tortuous diffusion path for a growth inhibitor chemical, wherein the divider isolates an anode and cathode from a recombinant microorganism. In one embodiment, the growth inhibitor chemical is a reactive oxygen species and/or nitric oxide. In another embodiment, the porous divider comprises a membrane or solid porous material. In a specific embodiment, the divider comprise ceramic.

The disclosure also provides a method of producing a biofuel, comprising culturing a microorganism of any of the foregoing embodiments under conditions and in the presence or a suitable carbon source and reducing agent and isolating the biofuel. In one embodiment, the biofuel is isobutanol. In another embodiment, the reducing agent is formate or H₂. In yet a further embodiment, the microorganism is obtained from a Ralstonia sp. parental organism.

The disclosure also provides a bioreactor system comprising a source of H₂ or formate, a source of energy to generate H₂ or a combination thereof, a source of CO₂ and a recombinant microorganism of the disclosure. In one embodiment, the disclosure can comprise a light source for photosynthesis.

### DESCRIPTION OF THE FIGURES

FIG. 1A-C shows the design of Ralstonia eutropha cells as the biocatalyst in the process of electricity storage. (a) Schematic presentation of the energy conversion and carbon flow route of the overall process. CBB cycle, Calvin-Benson-Bassham cycle; ETC, electron transportation chain; MBH, membrane-bound hydrogenase; SH, soluble hydrogenase; FDH, formate dehydrogenase. (b) Engineered metabolic pathways from CO₂ to fuels in the context of the host's metabolic network. RuBP, Ribulose-1,5-bisphosphate; 3PGA, 3-phospho-D-glycerate; 2PGA, 2-phospho-D-glycerate; PEP, phosphoenolpyruvate; PHB, poly[R-(−)-3-hydroxybutyrate]; AHAS, acetohydroxy-acid synthase; KDC, 2-keto-acid decarboxylase; ADH, alcohol dehydrogenase. (c) Shows a general schematic of the overall system of the disclosure.

FIG. 2A-G shows the construction of a synthetic isobutanol and 3-methyl-1-butanol production pathway in Ralstonia eutropha. (a) isobutanol and isobutyraldehyde formation by the synthetic Ehrlich cassette. The 2-ketoacid decarboxylase (KDC) encoded by kivd of Lactococcus lactis was overexpressed in combination with different alcohol dehydrogenases (ADHs) encoded by adhA (L. lactis), adh2 (Saccharomyces cerevisiae), and yqhD (Escherichia coli), respectively. (b) Heterotrophic isobutanol and 3-methyl-1-butanol (3MB) production from 4 g/L fructose in German minimal medium using H16, LH75, and LH67 strains transformed with a plasmid harboring the kivd and yqhD overexpression cassette. LH106 is the strain resulted from LH75 transformed with the kivd and yqhD plasmid. LH74 is the strain resulted from LH67 transformed with the kivd and yqhD plasmid. (c) Construction of LH75 strain. Integration of the phaC1 promoter in front of the R. eutropha ilvBHC operon and ilvD gene to enhance branched-chain amino acid biosynthesis. (d) Construction of LH67 strain. Integration of alsS (Bacillus subtilis), ilvC (E. coli), and ilvD (E. coli) in R. eutropha genome. The AHAS (acetohydroxy-acid synthase, encoded by ilvBH or alsS) (e), IlvC (f), and IlvD (g) specific activities in vitro as measured using cell extract of wildtype H16, LH75 and LH67. Error bars indicate standard deviation (n=3).

FIG. 3A-C shows autotrophic higher alcohol production by the engineered Ralstonia strain. (a) Construction of the production strain LH74D. (b) Biofuel production performance by LH74D from CO₂ using electrolysis generated H₂ as the sole energy source. (c) Biofuel production performance by LH74D using formic acid as the sole carbon and energy source. Error bars indicate standard deviation (n=3).

FIG. 4A-F shows an integrated electro-microbial process for biofuel production from electricity and CO₂. (a) Schematic presentation showing the in situ electrochemical CO₂ reduction (and H₂O splitting) coupled with biofuel production by the engineered Ralstonia eutropha strain. (b) Transient inhibitory effect of in situ electrolysis on the growth of E. coli cells. (c) The induction of Ralstonia katG, sodC, and NorA promoters in electrolysis conditions. The katG, sodC, and NorA promoters are induced by hydrogen peroxide (H₂O₂), superoxide free radicals (O²⁻) and nitric oxide (NO), respectively. The promoters are used to drive the expression of the lacZ reporter gene. And the promoter activities are measured by the β-galactosidase assay. Error bars indicate standard deviation (n=3). (d) The configuration of the electromicrobial bioreactor. The cathode and the anode form concentric cylinders. The porous ceramic cup separates the two electrodes. (e) Biofuel production by the LH74 strain in the integrated electro-microbial process. Error bars indicate standard deviation (n=3). (f) shows a bioreactor of the disclosure.

FIG. 5 depicts a nucleic acid sequence (SEQ ID NO:1) derived from a kivd gene encoding a kdc polypeptide having 2-keto-acid decarboxylase activity.

FIG. 6 depicts a nucleic acid sequence (SEQ ID NO:3) derived from a PDC6 gene encoding a polypeptide having 2-keto-acid decarboxylase activity.

FIG. 7 depicts a nucleic acid sequence (SEQ ID NO:5) derived from an ARO10 gene encoding a polypeptide having 2-keto-acid decarboxylase activity.

FIG. 8 depicts a nucleic acid sequence (SEQ ID NO:7) derived from a THI3 gene encoding a polypeptide having 2-keto-acid decarboxylase activity.

FIG. 9 depicts a nucleic acid sequence (SEQ ID NO:9) derived from a pdc gene encoding a polypeptide having 2-keto-acid decarboxylase activity.

FIG. 10 depicts a nucleic acid sequence (SEQ ID NO:11) derived from an ADH2 gene encoding a polypeptide having alcohol dehydrogenase activity.

FIG. 11 depicts a nucleic acid sequence (SEQ ID NO:13) derived from an ilvI gene encoding a polypeptide having acetolactate synthase large subunit activity.

FIG. 12 depicts a nucleic acid sequence (SEQ ID NO:15) derived from an ilvH gene encoding a polypeptide having acetolactate synthase small subunit activity.

FIG. 13 depicts a nucleic acid sequence (SEQ ID NO:17) derived from an ilvC gene encoding a polypeptide having acetohydroxy acid isomeroreductase activity.

FIG. 14 depicts a nucleic acid sequence (SEQ ID NO:19) derived from an ilvD gene encoding a polypeptide having dihydroxy-acid dehydratase activity.

FIG. 15 depicts a nucleic acid sequence (SEQ ID NO:21) derived from an ilvA gene encoding a polypeptide having threonine dehydratase activity.

FIG. 16 depicts a nucleic acid sequence (SEQ ID NO:23) derived from a leuA gene encoding a polypeptide having 2-isopropylmalate synthase activity.

FIG. 17 depicts a nucleic acid sequence (SEQ ID NO:25) derived from a leuB gene encoding a polypeptide having beta-isopropylmalate dehydrogenase activity.

FIG. 18 depicts a nucleic acid sequence (SEQ ID NO:27) derived from a leuC gene encoding a polypeptide having isopropylmalate isomerase large subunit activity.

FIG. 19 depicts a nucleic acid sequence (SEQ ID NO:29) derived from a leuD gene encoding a polypeptide having isopropylmalate isomerase small subunit activity.

FIG. 20 depicts a nucleic acid sequence (SEQ ID NO:31) derived from a cimA gene encoding a polypeptide having alpha-isopropylmalate synthase activity.

FIG. 21 depicts a nucleic acid sequence (SEQ ID NO:33) derived from an ilvM gene encoding a polypeptide having acetolactate synthase large subunit activity.

FIG. 22 depicts a nucleic acid sequence (SEQ ID NO:35) derived from an ilvG gene encoding a polypeptide having acetolactate synthase small subunit activity.

FIG. 23 depicts a nucleic acid sequence (SEQ ID NO:37) derived from an ilvN gene encoding a polypeptide having acetolactate synthase large subunit activity.

FIG. 24 depicts a nucleic acid sequence (SEQ ID NO:39) derived from an ilvB gene encoding a polypeptide having acetolactate synthase small subunit activity.

FIG. 25 depicts a nucleic acid sequence (SEQ ID NO:41) derived from an adhE2 gene encoding a polypeptide having alcohol dehydrogenase activity.

FIG. 26 depicts a nucleic acid sequence (SEQ ID NO:43) derived from a Li-cimA gene encoding a polypeptide having alpha-isopropylmalate synthase activity.

FIG. 27 depicts a nucleic acid sequence (SEQ ID NO:45) derived from a Li-leuC gene encoding a polypeptide having isopropylmalate isomerase large subunit activity.

FIG. 28 depicts a nucleic acid sequence (SEQ ID NO:47) derived from a Li-leuD gene encoding a polypeptide having isopropylmalate isomerase small subunit activity.

FIG. 29 depicts a nucleic acid sequence (SEQ ID NO:49) derived from a Li-leuB gene encoding a polypeptide having beta-isopropylmalate dehydrogenase activity.

FIG. 30 depicts a nucleic acid sequence (SEQ ID NO:51) derived from a pheA gene encoding a polypeptide having chorismate mutase P/prephenate dehydratase activity.

FIG. 31 depicts a nucleic acid sequence (SEQ ID NO:53) derived from a TyrA gene encoding a polypeptide having chorismate mutase T/prephenate dehydratase activity.

FIG. 32 depicts a nucleic acid sequence (SEQ ID NO:55) derived from an alsS gene encoding a polypeptide having acetolactate synthase activity.

FIG. 33A-B depicts a nucleic acid sequence (SEQ ID NO:57) of the operon fdsGBACD which encodes Ralstonia eutropha H16 soluble formate dehydrogenase complex.

FIG. 34 depicts a nucleic acid sequence (SEQ ID NO:63) of the operon hoxKGZ, which encodes Ralstonia eutropha H16 membrane-bound hydrogenase complex.

FIG. 35A-B depicts a nucleic acid sequence (SEQ ID NO:67) of operon hoxFUYH which encodes Ralstonia eutropha H16 soluble hydrogenase complex.

### DETAILED DESCRIPTION

As used herein and in the appended claims, the singular forms “a,” “and,” and “the” include plural referents unless the context clearly dictates otherwise. Thus, for example, reference to “a microorganism” includes a plurality of such microorganisms and reference to “the polypeptide” includes reference to one or more polypeptides known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Photovoltaic cells harvest energy from sunlight and generate electricity with relatively high energy efficiencies, typically ranging from 10 to 20%. However, due to the diffuse and intermittent nature of solar energy, the electricity produced by photovoltaics needs to be efficiently stored. The current methods of electricity storage via batteries suffer from low energy density, which generally ranges between 0.1-0.7 MJ/kg (or 0.5-2.0 MJ/L). Alternatively, electrolytic water splitting stores electrical energy in chemical bonds in H₂ molecules with high efficiencies. However, H₂ utilization in the transportation sector faces many engineering challenges. Compared to H₂, formic acid would be a favorable energy carrier at the interface between electrolysis and microbial cells. Electrochemical production of formic acid from CO₂ and H₂O has been extensively studied and can achieve relatively high current efficiencies.

The solar electricity-powered water splitting in effect achieves the “light reaction” of biological photosynthesis in that they both convert solar energy to chemical reducing energy, in the form of H₂.

Some lithoautotrophic microorganism can utilize H₂ to generate NADH and ATP and to power CO₂ fixation in the CBB cycle, the same series of reactions in the “dark reaction” of photosynthesis. The fixation of CO₂ into longer chain chemicals suitable for use as liquid fuels requires (1) formation of C—C bond, and (2) reduction of carbon. In plants and photosynthetic microorganisms, CO₂ fixation (the dark reaction) is coupled with the light reaction of photosynthesis, which produces the reducing power (NADPH) and energy (ATP). However, in various photosynthetic systems light penetration in culture environments can be limiting, reducing efficiency and fuel production.

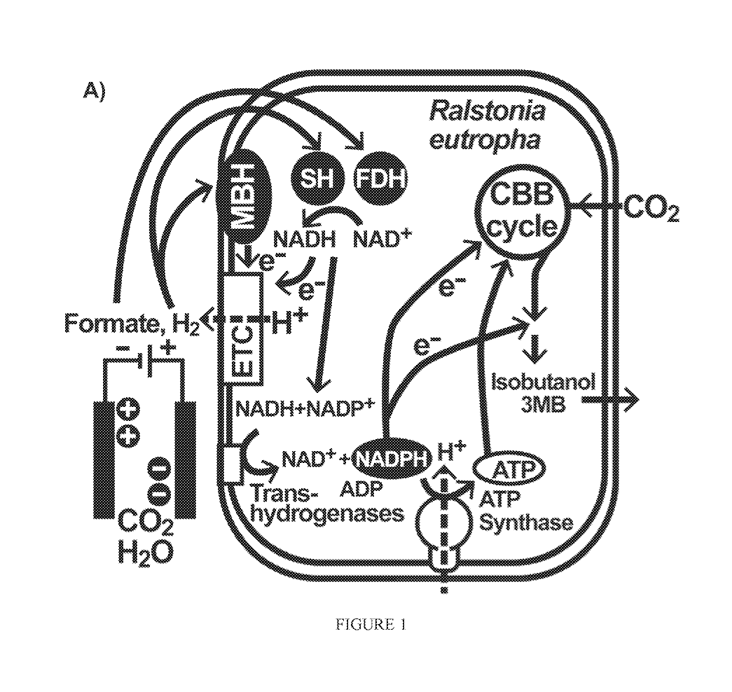

Nature has evolved organisms that have decoupled the photosynthesis process required for producing reducing power. A group of microbes derive energy and reducing power from chemicals (chemoautotrophs) such as formate, or inorganics (lithoautotrophs) such as H₂, to drive CO₂ fixation. Examples of these organisms include Ralstonia (formerly Alcaligenes) and Xanthobacter. In particular, Ralstonia eutropha has been extensively studied for the production of polyhydroxyalkanoate (PHA) industrially. It is metabolically active and versatile, and grows reasonably fast. During lithotrophic growth, molecular H₂ is oxidized by a membrane-bound hydrogenase (MBH) and a soluble hydrogenase (SH), and formate is metabolized by a soluble formate dehydrogenase (FDH; encoded by SEQ ID NO:57) to provide R. eutropha with the reducing power, which then drives the Calvin-Benson-Bassham (CBB) cycle and other metabolic pathways (FIG. 1A). Ralstonia can use either H₂ or formate to drive CO₂ fixation through the Calvin-Benson-Bassham (CBB) cycle. These organisms have hydrogenases and formate dehydrogenase to derive NAD(P)H from H₂ and formate, respectively. Thus, the NAD(P)H and ATP that are needed to drive CO₂ fixation are obtained either via the CBB or rTCA cycles. For example, NADH can be derived from H₂ via hydrogenases or formate via formate dehydrogenases. NADH can then be converted to NADPH via transhydrogenases. ATP is generated via the electron transport chain using O₂ as the terminal electron acceptor.

Formate is highly soluble and is readily converted to both carbon dioxide and NADH in a stoichiometric ratio by formate dehydrogenase in the cells, circumventing the poor mass transfer issue of both CO₂ and H₂ as gas substrates. However, the high solubility of formic acid increases the cost of product separation from electrochemical process. If not separated effectively, accumulated formate can be decomposed at the anode, reducing the yield of the process. As such, an integrated process featuring simultaneous electrochemical formate production and biological formate utilization is desirable, since the costly product separation could be circumvented and no formate accumulation would occur. When producing compounds more reduced than formate, such as higher alcohols, more reducing power than CO₂ is required. Thus, excess CO₂ will be released by the microbes, which provide dissolved CO₂ in the vicinity of the working electrode to be reduced electrochemically. Using the product of CBB cycle as a precursor, carbon chains with various lengths, conformations, and functionalities can be synthesized. Therefore, a hybrid process comprised of the man-made “light reaction” to generate H₂ or formate, and the biological “dark reaction” to store electricity in the C—C bonds of liquid fuels, bioreactors useful for liquid fuel production, and recombinant microorganisms are provided by the disclosure.

The disclosure provides an integrated process for production of liquid fuel from electricity includes (1) metabolic engineering of a photoautotrophic, chemoautotrophic or lithoautotrophic organism to produce liquid fuels, (2) electrochemical production of a reducing agent such as H₂ or formate using, e.g., photovoltaics from water or CO₂, respectively, and (3) eliminating the adverse effect of electrolysis on microbial cells.

The disclosure provides a process that utilizes electrically generated reducing power (H₂ or formate) as an electron donor to drive the biological CO₂ reduction process. The H₂ or formate can be generated by electrolysis, which can be conducted in an integrated electro-biological process so that the electrolysis rate can match the biological rate. Since the biological consumption of H₂ or formate is relatively small compared to electrolysis, the latter can run at a low current density and thereby increase the efficiency of electrolysis. By reducing the rate of electrolysis to match the biological rate, the current efficiency increased. Another major cost of electrolysis is product purification. In this integrated electro-bio process, the product (H₂ or formate) is introduced directly into the bioreactor with minimal or no purification to separate water.

H₂ and formate are used as exemplary reducing mediators by recombinant microorganism of the disclosure. H₂ can be transferred to the microbes, and the reducing power can be extracted by hydrogenase to drive the CO₂ fixation process. Formate can also be taken up by cells and produce NAD(P)H and CO₂ by formate dehydrogenase. NAD(P)H is then used to drive CO₂ fixation. O₂ is chosen as the terminal electron acceptor, as it is most environmentally friendly.

In addition, H₂ and formate under low O₂ conditions also reduces the oxidative loss (a.k.a. photorespiration) of ribulose-1,5-bisphosphate carboxylase oxygenase (RuBisCO), the enzyme used by the CBB cycle for CO₂ assimilation. The oxidative loss is an intrinsic problem of RuBisCO, and defies millions of years of evolution and decades of protein engineering. Since we need O₂ as an electron acceptor for generating ATP, low O₂ condition is ideal

This disclosure demonstrates that alternative reducing processes, other than photosynthesis light reactions, can be used. For example, H₂, formate and electricity can be used instead of photosynthesis to deliver chemical reducing power to drive CO₂ fixation using the Calvin-Benson-Bassham (CBB) cycle and the biosynthesis of higher alcohols such as, for example, isobutanol and 3-methyl-1-butanol (3MB).

The overall reaction of CO₂ fixation to isobutanol via the CBB cycle is calculated as follows:

6CO₂+12NADPH+14ATP→Isobutanol+12NADP+14 ADP+2CO₂

The ATP expenditure is slightly better than the CO₂ production to glucose on a per carbon basis.

As one aspect of the disclosure, the disclosure provides recombinant microorganisms and engineered metabolic pathways for microbial production of higher alcohols. These pathways can be engineered into various microbial host cells as identified elsewhere herein, but include, for example, E. coli, Saccharomyces cerevisiae, Bacillus subtilis, Clostridia, Ralstonia (formerly Alcaligenes), Xanthobacter and Corynebacteria. The disclosure describes, in one embodiment, the engineering of lithoautotrophic microorganism, Ralstonia eutropha, as the production organism, which can fix CO₂ in the dark using H₂ or formate as the energy source, to generate branched chain alcohols such as isobutanol and 3-methyl-1-butanol (3MB), as the target products. Isobutanol and 3MB have energy densities of 36.1 and 37.7 MJ/kg (or 29.0 and 30.5 MJ/L), respectively, which are two orders of magnitude higher than that of batteries.

The disclosure provides methods and compositions for the production of higher alcohols using a culture of microorganisms that utilizes CO₂ as a carbon source and utilizes a non-light or light and non-light produced reducing agent for production of NADPH (e.g., chemoautotrophs, lithoautotrophes, photoautotrophs and any combination thereof). For example, the cyanobacterium, S. elongates, can be engineered to accept H₂ and formate as electron donors, and to decouple the CBB cycle from the light reaction. An advantage of cyanobacteria is that they can also harvest sun light and thus can use photosynthesis wherever light is available and use reducing mediator whenever or wherever light is unavailable. This strategy allows the organism to use solar energy directly or indirectly through mediators and solves the problem of large light area requirement of photosynthesis. Another advantage of cyanobacteria is that synthesis of isobutanol and isobutyraldehyde can be achieved in relatively high productivity.

For example, CO₂ is converted to pyruvate, which is then converted to isobutanol via the keto acid pathway (FIG. 1B). AlsS (from B. subtilis) and ilvCD (from E. coli), and kivd (from Lactococcus lactis) are the most effective in producing isobutanol and isobutyraldehyde, from keto acids and can be readily expressed in multiple organisms. These genes, among others, can be used to achieve isobutanol production.

Although the utilization of H₂ and formate as an electron donor to drive CO₂ fixation has been described in lithoautotrophic and chemoautotrophic organisms, these organisms are poorly characterized and no attempts have been reported to alter their metabolic pathways to produce fuels. The disclosure uses as examples three organisms, cyanobacteria Synechococcus elongatus, Ralstonia eutropha, and Rhodopseudomonas palustris as engineered organism to demonstrate the invention. Cyanobacteria cannot fix CO₂ in the dark, and no attempts were reported to engineer cyanobacteria to utilize H₂ or formate as an energy source. Ralstonia have been used to produce polyhydroxyalkanoate (PHA), which is a biodegradable polymer, from sugars. This organism is metabolically versatile, and can utilize H₂ and formate as an electron source for CO₂ fixation. But no attempt has been made to use CO₂ for synthesis of chemicals, polymers, or fuels in this organism. Rh. palustris is also metabolically versatile and can fix CO₂ using the CBB pathway.

The CBB cycle is the most common and best studied pathway for CO₂ fixation. However, its energy expenditure is the highest, because it uses the high energy phospho-group to activate intermediates. Other competing pathways include the Wood-Ljundahl (reductive acetyl coA) pathway, the reductive TCA cycle, the 3-hydroxypropionate (3HP)glyoxylate cycle, and the 3HP/4-hydroxybutyrate (4HP) cycle.

The overall reducing equivalent requirement and ATP equivalent requirement of each pathway are summarized in Table 1. Note that these pathways all have the same requirement for reducing equivalent, as it is dictated by the chemical structures of the substrate and the product. However, CBB and 3HP/glyoxylate are the most energy intensive, while the reductive TCA and Wood-Ljundahl pathways are most energy efficient. If the P/O ratio is assumed to be 2, the total reducing equivalent required by using CBB, pathway is 19, while the reduced TCA or Wood-Ljundahl pathways use 14 and 13 total reducing equivalents, respectively. The energy saving by using these more efficient pathways amounts to 26-30%.

Since all known Woods-Ljungdahl pathway enzymes are oxygen-sensitive, while some rTCA enzymes are oxygen-tolerant (Table 2), the rTCA cycle was chosen as the alternative CO₂ fixation pathway. This allows the use of O₂ as the electron sink, while maintaining an energy efficiency that is similar to the Woods-Ljungdahl pathway. However, aerobic rTCA organisms (Hydrogenobacter) are thermophiles and difficult to manipulate.

Once the rTCA cycle is reconstructed in E. coli, the host can be further engineered to synthesize isobutanol and to utilize H₂ or formate as an electron donor.

TABLE 1
Reducing equivalent “[H₂]” and ATP equivalent “~P” needed for
each CO₂ fixing pathway. “[H₂]” represents a two-electron donor,
such as NAD(P)H, Flavin-H₂, or 2 reduced Ferredoxins.
Total “[H₂]” = “[H₂]” + “~P”/2, with an assumption
that P/O ratio equals 2.

| Pathways | CO₂ | H₂CO₃ | “[H₂]” | “~P” | Total “[H₂]” |
|---|---|---|---|---|---|
| CBB | 6 | 0 | 12 | 14 | 19 |
| 3HP/glyoxylate | 0 | 6 | 12 | 14 | 19 |
| 3HP/4HB | 2 | 4 | 12 | 12 | 18 |
| reductive TCA | 6 | 0 | 12 | 4 | 14 |
| Wood-Ljundahl | 6 | 0 | 12 | 2 | 13 |

However, other pathways are typically used by thermophiles (Table 2).

TABLE 2
Comparison of different CO₂ fixation organisms

| | | litho/chemo | existing | growth | | O2 | doubling | | genetic | |
|---|---|---|---|---|---|---|---|---|---|---|
| Pathways | Organisms | autotrophic? | electon donor | temp | | sensitive? | time | | tools | Comments |
| CBB | | | | | | | | | | |
| | Synechococcus | to be engineered | photosynthesis | 30 | C. | no | 4 | h | available | produce isobtuanol |
| | elongatus | | | | | | | | | |
| | Ralstonia | yes | H2, Formate | 30 | C. | no | 5-10 | h | available | produce PHA |
| | eutropha | | | | | | | | | |
| Reductive TCA | | | | | | | | | | |
| | Hydrogenobacter | yea | H2 | 70 | C. | no | 15 | h | no | low density culture |
| | thermophilus | | | | | | | | | |
| | Chlorobium | yes | thiosulfate | 26-29 | C. | yes | 15-20 | h | no | low density culture |
| | limicola | | | | | | | | | |
| Wood-Ljundahl | | | | | | | | | | |
| | Moorella | yes | H2, formate | 55-60 | C. | somewhat | 15-20 | h | no | low density culture |
| | thermoacetica | | | | | | | | | |

For the above reasons, suitable hosts includes, for example, cyanobacteria, S. elongates and R. eutropha. R. eutropha can already use H₂ and formate as electron donors for CO₂ fixation, and has been used industrially for PHA synthesis. Its growth rate is acceptable and genetic tools are available. The isobutanol pathway genes (FIG. 1B) can be expressed in R. eutropha to produce isobutanol from CO₂ and H₂ and formate. S. elongates has been used for isobutanol production from CO₂ with high productivity. S. elongates can be engineered to use H₂ or formate as electron donors by expressing hydrogenase and formate dehydrogenase. The organism can also be engineered to further inactivate innate regulations that coordinate the light reaction with the dark reaction. The resulting organism can use either light or electron mediators (H₂ or formate) to drive isobutanol production from CO₂.

In one embodiment of the disclosure, the CBB pathway genes in a recombinant microorganism are amplified and deregulated so that they are not subject to transcription level or protein level control. The use of electron mediators in low O₂ environment also reduces photorespiration of Rubisco, which is a major efficiency loss in photosynthesis.

FIG. 1A shows a CO₂ fixation pathway to produce pyruvate via the CBB cycle. FIG. 1B shows a general pathway for production of isobutanol from pyruvate in a recombinant microorganism. Further, the metabolite 2-ketoisovalerate can be produced by a recombinant microorganism metabolically engineered to express or over-express enzymes encoded by ilvIHCD genes. This metabolite can then be used in the production of isobutanol or 3-methyl 1-butanol.

The rTCA cycle shares many enzymes with the oxidative TCA cycle, with the exception of four irreversible enzymes, namely ATP-citrate lyase (ACL), pyruvate:ferredoxin oxidoreductase (POR), 2-oxoglutarate:ferredoxin oxidoreductases (OGOR) and isocitrate dehydrogenase (ICDH). A soluble fumarate reductase (FRD), rather than a membrane-bound fumarate reductase as is found in E. coli, has been proposed to be functional in the rTCA cycle in Hydrogenobacter and thus might also be required to reverse the oxidative TCA cycle. The rTCA cycle does not use high-energy phosphate bonds to activate its carbon intermediates, and therefore, its energy cost is much lower. To produce one mole of pyruvate, it requires only 2 moles of ATP, in addition to reducing power. The disclosure also provides recombinant organisms overexpressing the irreversible enzymes and utilizing the reversible enzymes in E. coli, to reverse the direction of the TCA cycle in E. coli. Indeed, it has been reported that the oxidative and reductive TCA cycles coexist in the symbiont of the deep-sea tube worm Riftia pachyptila (Fisher and Girguis, 2007) and can be coordinated under a different physiological status.

The rTCA cycle is a common mechanism used by bacteria dwelling in hot springs and deep-sea thermal vents (Fisher and Girguis, 2007; Hall et al., 2008). While the rTCA cycle is more commonly seen in anaerobic bacteria, it also exists in aerobic bacteria, such as Hydrogenobacter thermophilus TK-6 (Shiba et al., 1985). Because of this oxygen tolerance, the rTCA cycle genes in E. coli can be cloned to take advantage of the well-characterized and highly active E. coli metabolic systems. S. elongatus does not utilize H₂ or formate as an electron donor. As described herein, S. elongatus can be engineered to utilize these electron sources and alter its innate regulation networks to fix CO₂ in the dark. On the other hand, Ra. eutropha and Rh. palustris are able to utilize H₂ or formate as electron sources to fix CO₂ in the dark. As further described herein, the microorganism can be engineered to channel the metabolic flux to isobutanol in an efficient way.

The proteobacterium Ralstonia eutropha possesses two energy-linked (NiFe) hydrogenases: a membrane hydrogenase and a cytoplasmic hydrogenase. The membrane hydrogenase is involved in electron transport-coupled phosphorylation through coupling to the respiratory chain, whereas the cytoplasmic hydrogenase is able to reduce NAD⁺ to generate reducing equivalents (Schink et al., Biochim. Biophys. Acta 567:315-324, 1979; Schneider et al. Biochim. Biophys. Acta 452:66-80, 1976, each of which is incorporated herein by reference in its entirety). The genes encoding the two hydrogenases are clustered in two separate operons together with regulatory genes involved in hydrogenase biosynthesis on megaplasmid pHG1 (Schultz et al. Science 302:624-627, 2003; Schwartz et al. J. Bacteriol. 180:3197-3204, 1998, each of which is incorporated herein by reference in its entirety). A third hydrogenase was identified in R. eutropha and classified as belonging to the subclass of H₂-sensing (NiFe) hydrogenases (Kleihues et al., J. Bacteriol. 182:2716-2724, 2000, incorporated herein by reference in its entirety). The third hydrogenase is stable in presence of O₂, CO, and C₂H₂. The rate of hydrogen oxidation of this third hydrogenase is one to two orders of magnitude lower than that of standard membrane and cytoplasmic hydrogenase. The third hydrogenase contains an active size similar to the initial two hydrogenases. This third hydrogenase is encoded by the hoxB and hoxC genes (large and small subunit, respectively). The hyp genes (hypA1B1F1CDEX) are responsible for the maturation of the third hydrogenase in R. eutropha are located between the membrane hydrogenase genes and hoxA.

Oxygen-tolerant hydrogenases have been identified in Bradyrhizobium japonicum (Black et al., 1994), Ra. eutropha (Buhrke et al., 2005; Lenz and Friedrich, 1998), Rhodobacter capsulatus (Elsen et al., 1996; Vignais et al., 2002), Thiocapsa roseopersicina (Kovacs et al., 2005), and Rh. palustris (Rey et al., 2006). Significant heterologous activity of one these hydrogenases has been reported in Synechococcus elongatus PCC7002, with the chromosomal integration of the soluble hydrogenase and accessory maturation proteins of Ra. eutropha (Xu, 2009).

In a specific embodiment, a microorganism which naturally contains a CO₂ fixation enzyme and an ability to use H₂ or formate for reduction is engineered to produce an alcohol. In one embodiment, the alcohol is isobutanol. In another embodiment, the recombinant microorganism is engineered from a Ralstonia sp. to contain a pathway comprising the enzymes and conversion set forth in the following tables. The following tables set forth reaction pathways for various recombinant microorganism of the disclosure including a list of exemplary genes and homologs and organism source.

E. coli has three hydrogenases, of which at least one hydrogenase has been shown to be reversible (Maeda et al., 2007). By using the native reversible hydrogenase of E. coli under high pressure of hydrogen in the culture or by overexpressing hydrogenases from other species (eg. Ra eutropha), we can harness the power of hydrogenase to use hydrogen as an energy source.

Examples of microorganisms that utilize CO₂ as a carbon source include photoautotrophs, chemoautotrophs and lithoautotrophs. In some embodiments, the methods and compositions of the disclosure comprise a single culture or co-culture of autotrophs, photoautotrophs and a photoheterotroph or a photoautotroph and a microorganism that cannot utilize CO₂ as a carbon source.

In any of the embodiments described herein, the microorganism can be a chemoautotrophs, photoautotroph or lithoautotroph comprising the ability to reduce CO₂ in the dark to a metabolite that can be used for producing a biofuel. In yet another embodiment, the microorganism of any of the foregoing comprises an innate ability to fix CO₂ using H₂ or formate as a source for the production of NAD(P)H. In another embodiment, the microorganism is R. eutropha. In yet another embodiment, the disclosure provides a recombinant microorganism that has been engineered to utilize H₂ or formate as an electron donor for producing NAD(P)H and fixing CO₂. For example, S. elongatus does not utilize H₂ or formate as an electron donor; accordingly in this embodiment, the recombinant microorganisms comprises an engineered pathway (e.g., comprising a hydrogenase or formate dehydrogenase) to utilize H₂ or formate as an electron donor. For example, a microorganism of the disclosure can be engineered to express a formate dehydrogase having at least 50-100% identified to a polypeptide encoded by a sequence of SEQ ID NO:57 and having formate dehydrogenase activity. Alternatively, or in addition, the recombinant microorganism of the disclosure can be engineered to express a hydrogenase having at least 50-100% identity to a polypeptide encoded by SEQ ID NO: 63 or 67 and having hydrogenase activity. In one embodiment, S. elongatus is engineered to utilize these electron sources and alter its innate regulation networks to fix CO₂ in the dark. E. coli, for example, has three hydrogenases, of which at least one hydrogenase has been shown to be reversible. By using the native reversible hydrogenase of E. coli under high pressure of hydrogen in the culture or by overexpressing hydrogenases from other species (e.g., Ra. eutropha), E. coli can be engineered to harness the power of hydrogenase to use hydrogen as an energy source. In another embodiment, the microorganism is engineered to fix CO₂ (e.g., by engineering into the organism a CO₂ fixation enzyme such as RuBisCo or a homolog thereof). In this latter embodiment, the E. coli can be further engineered to express a CO₂ fixation enzyme and enzymes for the production of a desired biofuel.

Ribulose-1,5-bisphosphate carboxylase oxygenase, most commonly known by the shorter name RuBisCO, is an enzyme (EC 4.1.1.39) that is used in the Calvin cycle to catalyze the first major step of carbon fixation, a process by which the atoms of atmospheric carbon dioxide are made available to organisms in the form of energy-rich molecules such as sucrose. RuBisCO catalyzes either the carboxylation or the oxygenation of ribulose-1,5-bisphosphate (also known as RuBP) with carbon dioxide or oxygen.

RuBisCO is one of the most abundant proteins on Earth. Accordingly, a number of homologs and variants of RuBisCO have been identified and generated. RuBisCo usually consists of two types of protein subunit, called the large chain (L, about 55,000 Da) and the small chain (S, about 13,000 Da). The enzymatically active substrate (ribulose 1,5-bisphosphate) binding sites are located in the large chains that form dimers in which amino acids from each large chain contribute to the binding sites. A total of eight large-chain dimers and eight small chains assemble into a larger complex of about 540,000 Da. In some proteobacteria and dinoflagellates, enzymes consisting of only large subunits have been found.

Magnesium ions (Mg²⁺) are needed for enzymatic activity. Correct positioning of Mg²⁺ in the active site of the enzyme involves addition of an “activating” carbon dioxide molecule (CO₂) to a lysine in the active site (forming a carbamate). Formation of the carbamate is favored by an alkaline pH. The pH and the concentration of magnesium ions in the fluid compartment (in plants, the stroma of the chloroplast) increases in the light.

During carbon fixation, the substrate molecules for RuBisCO are ribulose 1,5-bisphosphate, carbon dioxide and water. RuBisCO can also allow a reaction to occur with molecular oxygen (O₂) instead of carbon dioxide (CO₂).

When carbon dioxide is the substrate, the product of the carboxylase reaction is a highly unstable six-carbon phosphorylated intermediate known as 3-keto-2-carboxyarabinitol 1,5-bisphosphate, which decays into two molecules of glycerate 3-phosphate. The 3-phosphoglycerate can be used to produce larger molecules such as glucose. When molecular oxygen is the substrate, the products of the oxygenase reaction are phosphoglycolate and 3-phosphoglycerate. Phosphoglycolate initiates a sequence of reactions called photorespiration, which involves enzymes and cytochromes located in the mitochondria and peroxisomes. In this process, two molecules of phosphoglycolate are converted to one molecule of carbon dioxide and one molecule of 3-phosphoglycerate, which can reenter the Calvin cycle. Some of the phosphoglycolate entering this pathway can be retained by plants to produce other molecules such as glycine. Some plants, many algae, and photosynthetic bacteria have overcome this limitation by devising means to increase the concentration of carbon dioxide around the enzyme, including C4 carbon fixation, crassulacean acid metabolism and using pyrenoid.

RuBisCO is usually active only during the day because ribulose 1,5-bisphosphate is not being produced in the dark, due to the regulation of several other enzymes in the Calvin cycle. In addition, the activity of RuBisCO is coordinated with that of the other enzymes of the Calvin cycle.

In plants and some algae, another enzyme, RuBisCO activase is used in the formation of the carbamate in the active site of RuBisCO. Ribulose 1,5-bisphosphate (RuBP) substrate binds more strongly to the active sites lacking the carbamate and markedly slows down the “activation” process. In the light, RuBisCO activase promotes the release of the inhibitory RuBP from the catalytic sites. CA1P binds tightly to the active site of carbamylated RuBisCO and inhibits catalytic activity. In the light, RuBisCO activase also promotes the release of CA1P from the catalytic sites. After the CA1P is released from RuBisCO, it is rapidly converted to a non-inhibitory form by a light-activated CA1P-phosphatase.

The removal of the inhibitory RuBP, CA1P, and the other inhibitory substrate analogs by activase requires the consumption of ATP. This reaction is inhibited by the presence of ADP, and, thus, activase activity depends on the ratio of these compounds in the chloroplast stroma. Furthermore, in most plants, the sensitivity of activase to the ratio of ATP/ADP is modified by the stromal reduction/oxidation (redox) state through another small regulatory protein, thioredoxin. In this manner, the activity of activase and the activation state of RuBisCO can be modulated in response to light intensity and, thus, the rate of formation of the ribulose 1,5-bisphosphate substrate.

In cyanobacteria, inorganic phosphate (Pᵢ) participates in the coordinated regulation of photosynthesis. Pᵢ binds to the RuBisCO active site and to another site on the large chain where it can influence transitions between activated and less active conformations of the enzyme. Activation of bacterial RuBisCO might be particularly sensitive to Pᵢ levels which can act in the same way as RuBisCO activase in higher plants.

The disclosure provides, in some embodiments, recombinant microorganisms that utilize upregulated RuBisCO to promote carbon fixation and alcohol production in photosynthetic organism as described herein, while comprising a recombinant non-light engineered redox pathway for NADPH production and utilization. For example, to maintain CBB gene expression at a high level, key enzymes such as RuBisCO, phosphoribulokinase (PRK), and glyceraldehyde-3-phosphate dehydrogenase (GAPDH) can also be constitutively overexpressed.

In order to engineer an organism of the disclosure to utilize formate as a reducing agent, formate dehydrogenases (FDHs) can be heterologously expressed in this certain microorganism. FDHs have been proven to be the most promising candidate for the development of NAD+ regeneration systems in organic synthesis for production of high-added-value products largely due to their wide pH-optimum (pH 6.0-9.0) and to the nonreversibility of enzymes (Burton, 2003; Hummel and Kula, 1989; Shaked et al., 1980; Wichmann and Vasic-Racki, 2005). Of the FDHs that have been studied, one from Candida boidinii is the most commonly used for the development of NAD+ regeneration systems (Ohshima et al., 1985). Studies on C. boidinii FDH have identified mutations that confer altered cofactor specificity (Rozzell, 2004), improved catalytic activity (Slusarczyk, 2003), and enhanced chemical stability (Slusarczyk, 2003; Felber, 2001).

Several FDHs have been integrated into the NSI site of S. elongatus PCC7942. The genes that encode the wild type and D195S/Y196H double mutant FDH from C. boidinii and the FDH from M. thermoacetica were each cloned into the NSI− targeting vector, under the IPTG-inducible Ptrc promoter. The D195S/Y196H double mutation was utilized because it results in a FDH with altered cofactor specificity from NAD(H) to NADP(H). The FDH gene from Moorella thermoacetica, encoded by Moth_{—}2314, has been indicated to encode for an enzyme with formate:NADP+ oxidoreductase activity. This enzyme was chosen because of its cofactor preference.

In addition to the FDHs, other genes were also heterologously expressed to optimize formate utilization. To ensure efficient formate uptake, a formate transporter encoded by focA from E. coli was also overexpressed. Furthermore, to specifically generate NADPH from formate oxidization, several transhydrogenases including pntAB and udhA from E. coli have been introduced in combination with wild type NAD+-dependent C. boidinii FDH. By using enzymatic assays of crude cyanobacterial cell lysates, as well as HPLC measurements of formate consumption in flask culture, co-expression of E. coli focA, C. boidinii wild type FDH, and E. coli pntAB enable S. elongatus to consume formate at a significant rate.

On the other hand, Ra. eutropha and Rh. palustris are able to utilize H₂ or formate as electron sources to fix CO₂ in the dark. In these organisms, a biofuel production pathway that converts pyruvate or other suitable intermediate into a biofuel (e.g., isobutanol) is engineered into these microorganisms.

As a chemolithoautotroph, Ra. eutropha is able to derive its energy and reducing power from inorganic compounds or elements, such as H₂ or formate, to drive CO₂ fixation through the CBB cycle. Ra. autropha is metabolically active and versatile, grows reasonably fast, and has been extensively studied for industrial production of polyhydroxyalkanoate (PHA) (Cramm, 2009; Pohlmann et al., 2006; Steinbüchel, 1992). Because of these characteristics, Ra. eutropha is a potential host for the conversion of CO₂ to isobutanol using H₂ or formate.

Ra. eutropha employs native hydrogen utilization pathways when it undergoes chemoautotrophic growth. Two types of hydrogen utilization pathways run in parallel to fuel the CO₂-fixing CBB cycle with ATP and NADPH: A membrane-bound hydrogenase (MBH), which oxidizes H₂ and feeds electrons into the respiratory chain to generate ATP; and also a soluble hydrogenase (SH), which directly uses NAD(P)+ as an electron acceptor to produce NAD(P)H at the expense of H₂. In addition, several transhydrogenases convert NADH into NADPH in order to meet the NADPH needs required by the CBB cycle (Cramm, 2009; Pohlmann et al., 2006). Ra. eutropha hydrogenases belong to a family of [NiFe] bidirectional hydrogenases. However, unlike most of the members in the family, which are sensitive to very low oxygen concentrations, Ra. eutropha hydrogenases are relatively oxygen tolerant, consistent with the aerobic physiological nature of this organism. This provides a great advantage and flexibility for strain manipulation and process optimization.

Similarly, formate can serve as both an electron donor and carbon source to sustain autotrophic growth of Ra. eutropha. A membrane-bound formate dehydrogenase oxidizes formate and transports the electrons into respiratory chain; and a soluble formate dehydrogenase uses NAD+ as the electron acceptor. The CO₂ produced from formate oxidization is then assimilated (Cramm, 2009; Pohlmann et al., 2006).

CO₂ is fixed through the CBB cycle in Ra. eutropha to pyruvate. To generate biofuels, genes that “hijack” the amino acid synthesis pathways can be used. For example, alsS from B. subtilis, ilvCD and yqhD from E. coli, and kivd from L. lactis (FIG. 5) can be engineered in Ra. eutropha to achieve autotrophic isobutanol synthesis.

To enhance isobutanol production efficiency, competing pathways that dissipate reducing equivalence or drain carbon flux need to be eliminated. In Ra. eutropha, a prominent example would be the PHA production pathway. The cells can naturally accumulate up to about 70% PHA (of the cell mass), even in autotrophic conditions with CO₂ and H₂ as substrates (Tanaka et al., 1995), which utilizes a large portion of carbon source and NADPH pools. Fortunately, the PHA production pathway is very well known and genetic manipulation tools to perform knock-out studies are available.

Rh. palustris is able to sense redox status and ATP levels, and is thus able to change metabolic modes according to changes in culture conditions (Larimer et al., 2004). The regulation mechanism is complicated and still not fully characterized. However, experimental evidence has shown that single-gene deletions of cbbRRS results in a significant reduction in total RuBisCO activity, which indicates that the cbbRRS is essential for RuBisCO expression (Romagnoli and Tabita, 2006). Therefore, in order to improve or maintain CBB cycle activity during different metabolic conditions, cbbRRS can be upregulated by overexpression or modify the PAS domains of cbbR to make it more efficient in catalyzing the phosphorylation cascade. This would hopefully result in the deregulation of the CBB cycle so that CBB cycle efficiency is improved in dark conditions.

The rTCA cycle shares many enzymes with the oxidative TCA cycle, with the exception of four irreversible enzymes, namely ATP-citrate lyase (ACL), pyruvate:ferredoxin oxidoreductase (POR), 2-oxoglutarate:ferredoxin oxidoreductases (OGOR) and isocitrate dehydrogenase (ICDH). In addition, a soluble fumarate reductase (FRD), rather than a membrane-bound fumarate reductase that is found in E. coli, is proposed to be functional in the rTCA cycle in Hydrogenobacter and thus can be useful to reverse the oxidative TCA cycle. Thus, to reverse the direction of the TCA cycle in E. coli, the following genes are heterologously express that encode for ACL, POR, OGOR, ICDH, and possibly FRD.

The rTCA cycle genes in Hydrogenobacter thermophilus TK-6 are the useful targets to clone into E. coli due to the fact that H. thermophilus utilizes its rTCA cycle under aerobic conditions. Despite the fact that these thermophilic enzymes are being expressed in mesophilic hosts, previous studies have shown that POR and OGOR from H. thermophilus are functional in E. coli (Ikeda et al., 2010; Yamamoto et al., 2010). In addition, heterologous expression of ACL from the thermophilic green sulfur bacteria, Chlorobium tepidum, results in activity in E. coli. Well-established enzyme assays (Ikeda et al., 2010; Yamamoto et al., 2010) will be used to test the activities of the overexpressed enzymes in vitro.

In addition, it is possible to test the enzyme activity by functional complementation. This complementation strategy is dependent upon the fact that the activity of phosphoenolpyruvate carboxykinase (Pck), NAD+-malate dehydrogenase (MaeA), NADP+-malate dehydrogenase (MaeB), or POR are necessary for growth when acetate is the sole carbon source (Oh et al., 2002). In E. coli, Pck, MaeA, and MaeB all have a role in the synthesis of pyruvate during gluconeogenic growth using acetate as the carbon source. Once POR is actively expressed, we can then overexpress both ACL and POR in the pckA maeA maeB mutant and then grow this strain with 2-ketoglutarate or glutamate as the carbon source. Growth will be observed if ACL is able to synthesize acetyl-CoA and POR is able to synthesize pyruvate for gluconeogenic growth.

Since the rTCA enzymes from H. thermophilus are thermophilic, these enzymes can be mutated for enhanced activity at 37 C. To do so, the functional complementation strategy described above can also serve as a selection strategy for directed evolution. Error-prone PCR will be used to generate mutations in the rTCA genes individually, and the library of the protein variants will be transformed into the pckA maeA maeB triple mutants. The more active mutants will support faster growth. Thus, after a few rounds of growth enrichment isolates of single colonies can be obtained to assay for enzymatic activity. The whole process will be repeated until sufficient activities of these enzymes are evolved.

The remaining genes necessary for the reconstitution of the rTCA cycle can be supplied by the reversible enzymes of the oxidative TCA cycle. These genes are regulated by multiple transcription networks, including the ArcA, Fnr, and cAMP-CRP systems. The regulatory pathways can be altered to ensure that the necessary genes are expressed and functional under electro-autotrophic conditions. In addition, the pckA maeA maeB mutant is expected to reduce the decarboxylation of TCA cycle intermediates and thus favor the rTCA direction.

For example, enzymes of Scheme I, below, may be engineered into these organisms to allow them to produce a biofuel from pyruvate. In yet another embodiment, competing pathways, such as the PHA or PHB pathway in R. eutropha, may be disrupted to improve the bioavailability of metabolites for the production of a biofuel (e.g., by increasing pyruvate levels). A metabolic feature of R. entropha is that it is one of the best-known natural polyhydroxyalkanoate (PHA) hyper-producers. PHA such as poly[R-(−)-3-hydroxybutyrate] (PHB) is produced as a storage compound and also as the metabolic sink for carbon and reducing equivalents. When PHB synthesis is disrupted, large amounts of pyruvate (the upstream substrate of PHB biosynthetic pathway) is secreted out of the cells, suggesting that the overall metabolic network is well-suited for pushing carbon and reducing power through this pathway at the pyruvate node. Thus, the keto acid pathways for isobutanol and 3MB production are well-positioned to channel both pyruvate and NADPH into biofuel production as the new metabolic sink.

In one embodiment, the disclosure provides a recombinant photoautotroph, chemoautotroph or lithoautotroph that has been engineered to produce a biofuel (e.g., isobutanol or 3-methyl-1-butanol) comprising overexpression an endogenous or expressing or over expressing a heterologous enzyme. The recombinant microorganism may further comprise a reduction or elimination of a competing pathway, wherein the reduction or elimination increases pyruvate production or other intermediate metabolites in biofuel production. In one embodiment, the lithoautotroph has a reduction or elimination in the production of poly[R-(−)-3-hydroxybutyrate] (PHB). For example, in one embodiment a polyhydroxyalkanoate synthase (E.C. 2.3.1.-) activity is reduced or eliminated thus redirecting the metabolic flux to an accumulation of pyruvate. In another embodiment, the organism comprises a reduction or elimination of activity of an enzyme selected from the group consisting of PhaA, PhaB, PhaC and any combination thereof (see, e.g., Scheme II).

In one embodiment, a recombinant microorganism of the disclosure comprises a chemoautotroph or lithoautotroph and a pathway as set forth in Scheme I and may further include a knockout of one or more enzymes in the pathway depicted in Scheme II. For example, a recombinant microorganism may comprise one or more heterologous enzymes identified as (1)-(9) or may include overexpression of one or more enzymes identified as (1)-(9) or a combination of one or more heterologous enzymes and overexpression of one or more endogenous enzymes identified as (1)-(9). Exemplary enzymes are also identified in Scheme I, but it will be recognized by one of skill in the art that homologs of the enzymes or modified or engineered enzymes may be used so long as they are capable of the conversion identified in Scheme I.

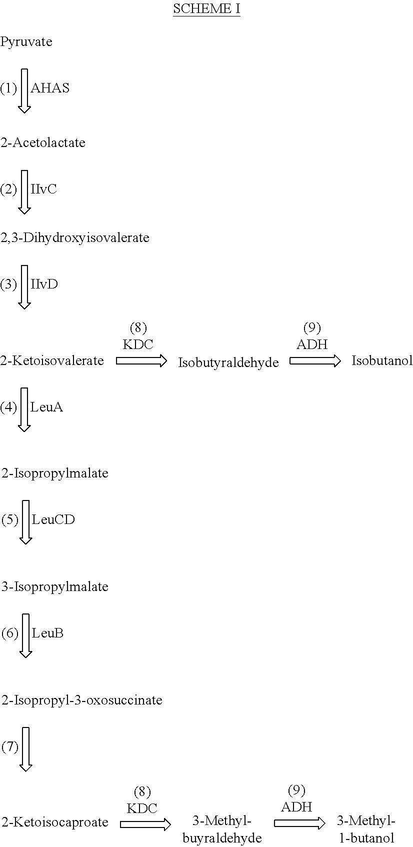

As mentioned above, a microorganism of the disclosure may inherently have a pathway that competes with a particular metabolite in the production of an alcohol (e.g., isobutanol or 3-methyl-1-butanol). Scheme II depicts one such pathway that is found in certain chemoautotrophs, photoautotrophs and lithoautotrophs (e.g., R. eutropha). Thus, a recombinant microorganism of the disclosure may comprise one or more knockouts of enzymes identified as (10)-(12) in Scheme II. Exemplary enzymes are also identified in Scheme II, but it will be recognized by one of skill in the art that homologs of the enzymes are encompassed so long as they are capable of the conversion identified in Scheme II. As will be readily apparent to one of skill in the art, the knocking out of one or more enzymes (10)-(12) of Scheme II will increase the level of pyruvate since the pyruvate can no longer be metabolized as set forth in Scheme II. The pyruvate is then readily available for metabolism using the pathway of Scheme I thereby increasing the metabolic flux to generate isobutanol, 3-methyl-1-butanol and related alcohols and intermediates. A metabolic feature of R. entropha is that it is one of the best-known natural polyhydroxyalkanoate (PHA) hyper-producers. PHA such as poly[R-(−)-3-hydroxybutyrate] (PHB) is produced as a storage compound and also as the metabolic sink for carbon and reducing equivalents. When PHB synthesis is disrupted, large amounts of pyruvate (the upstream substrate of PHB biosynthetic pathway) is secreted out of the cells, suggesting that the overall metabolic network is well-suited for pushing carbon and reducing power through this pathway at the pyruvate node. Thus, the keto acid pathways for isobutanol and 3MB production are well-positioned to channel both pyruvate and NADPH into biofuel production as the new metabolic sink.

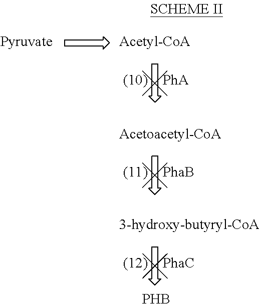

To achieve high titer levels of isobutanol production, it is beneficial to isolate a mutant that has a higher tolerance to isobutanol. The gram-negative Ra. eutropha appears to have comparable solvent tolerance to that of E. coli. Given the previous success in developing and characterizing E. coli strains that can tolerate up to 8 g/L isobutanol, similar mutagenesis approaches can be utilized in addition to solvent challenging selection. Furthermore, based on high-throughput genomic DNA sequencing of the solvent tolerant strains generated by our group as well as others, rational strain engineering approaches may also become available.

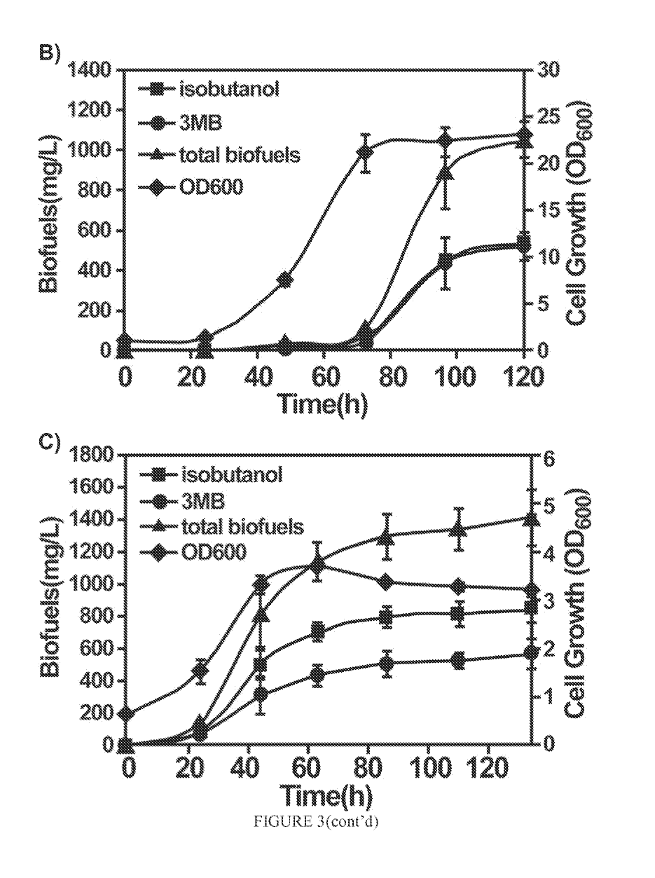

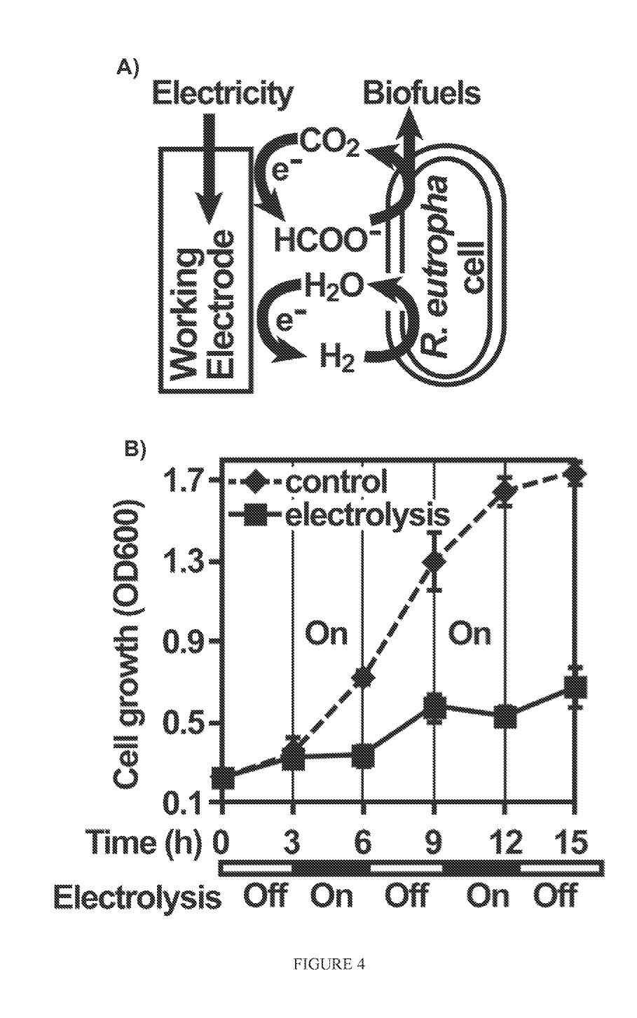

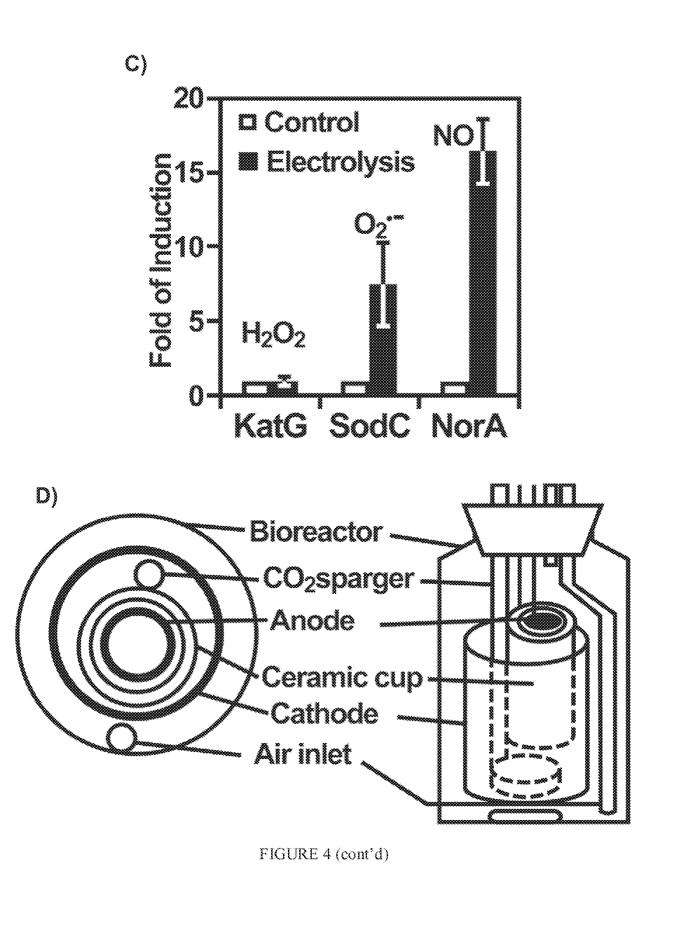

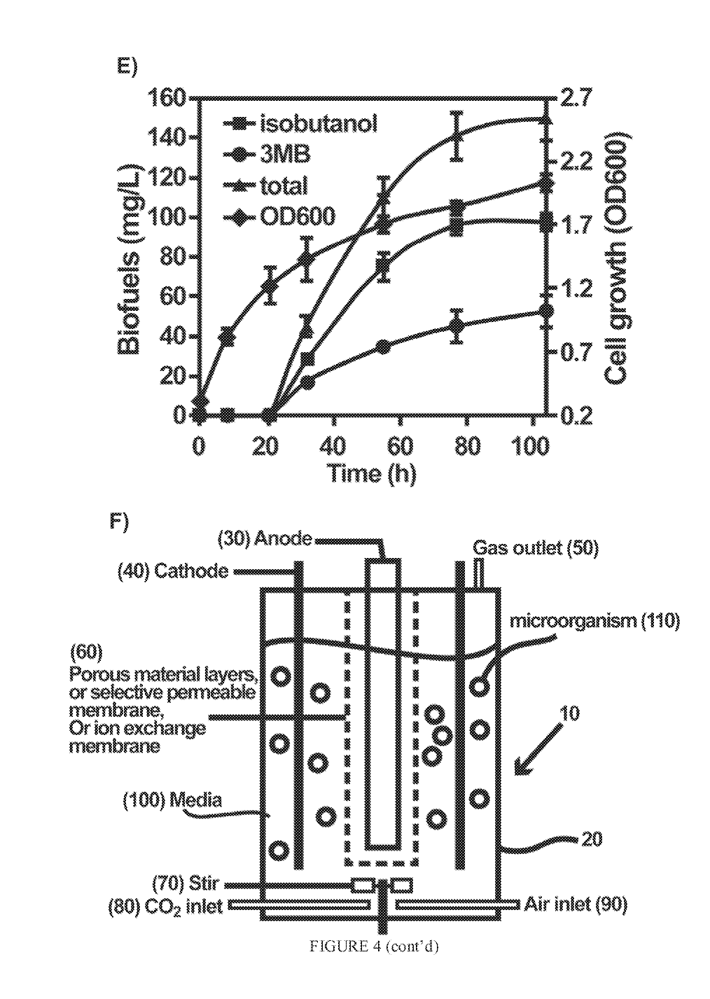

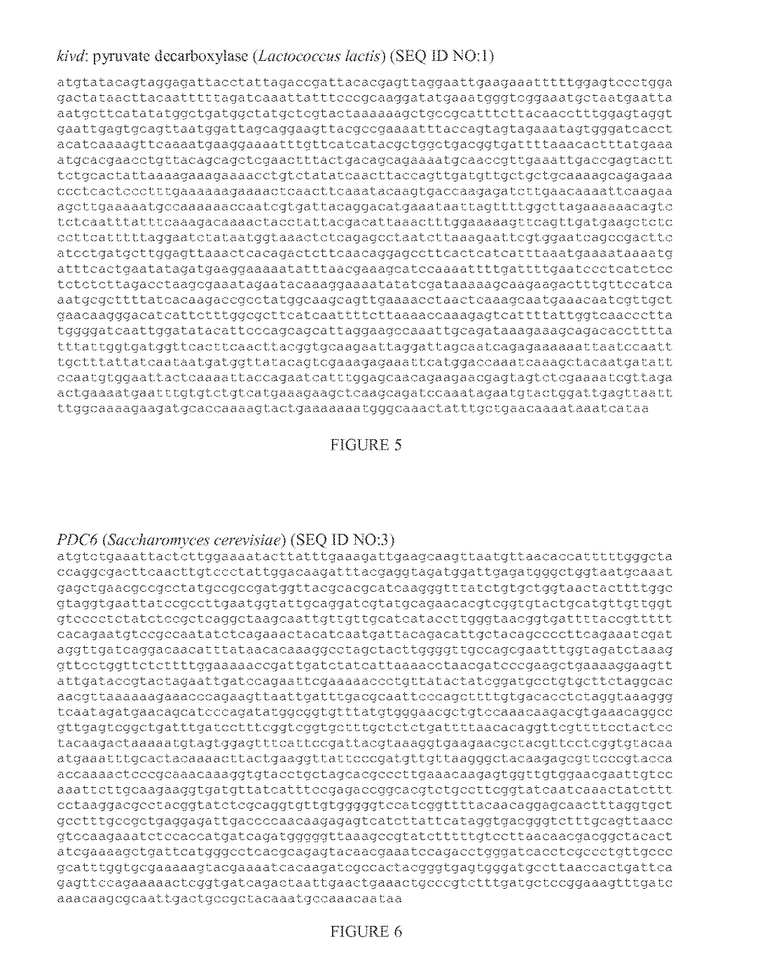

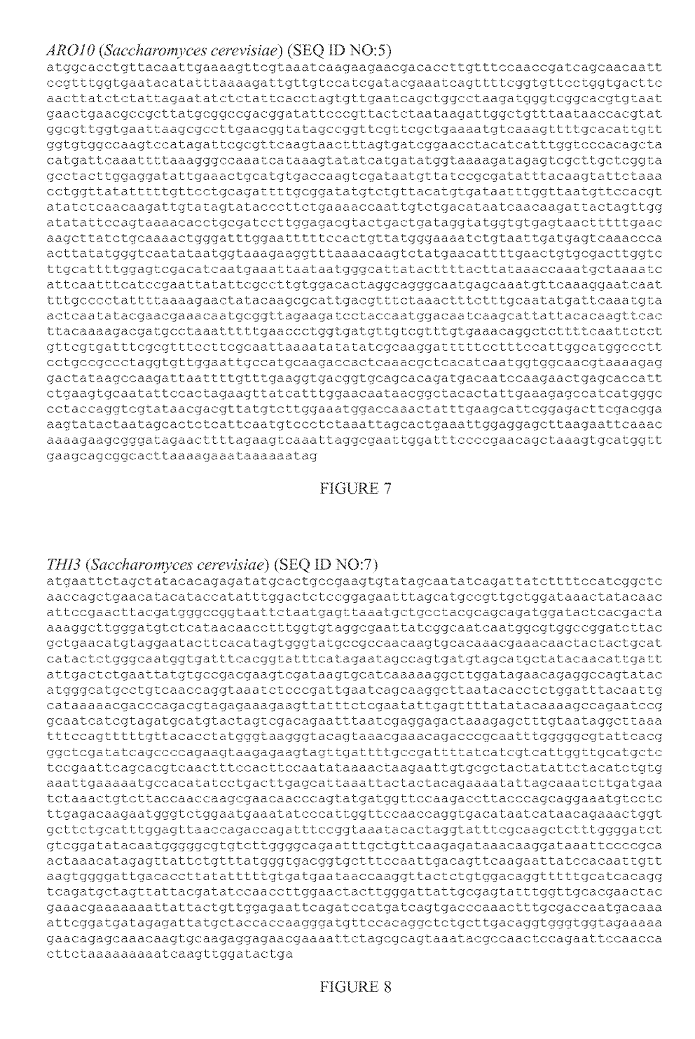

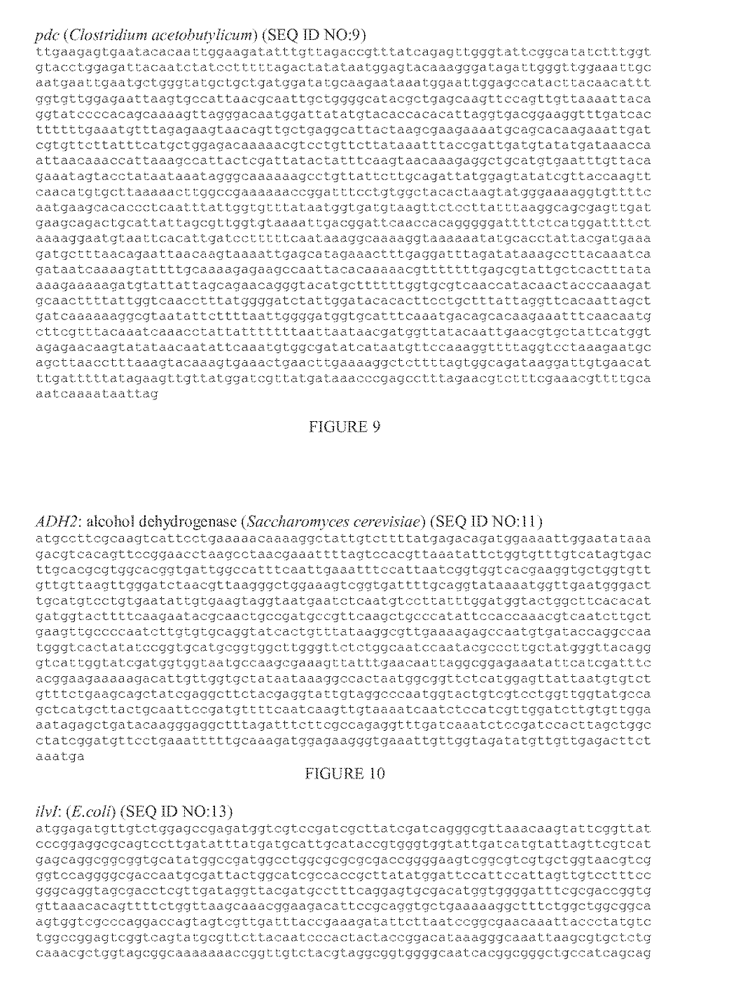

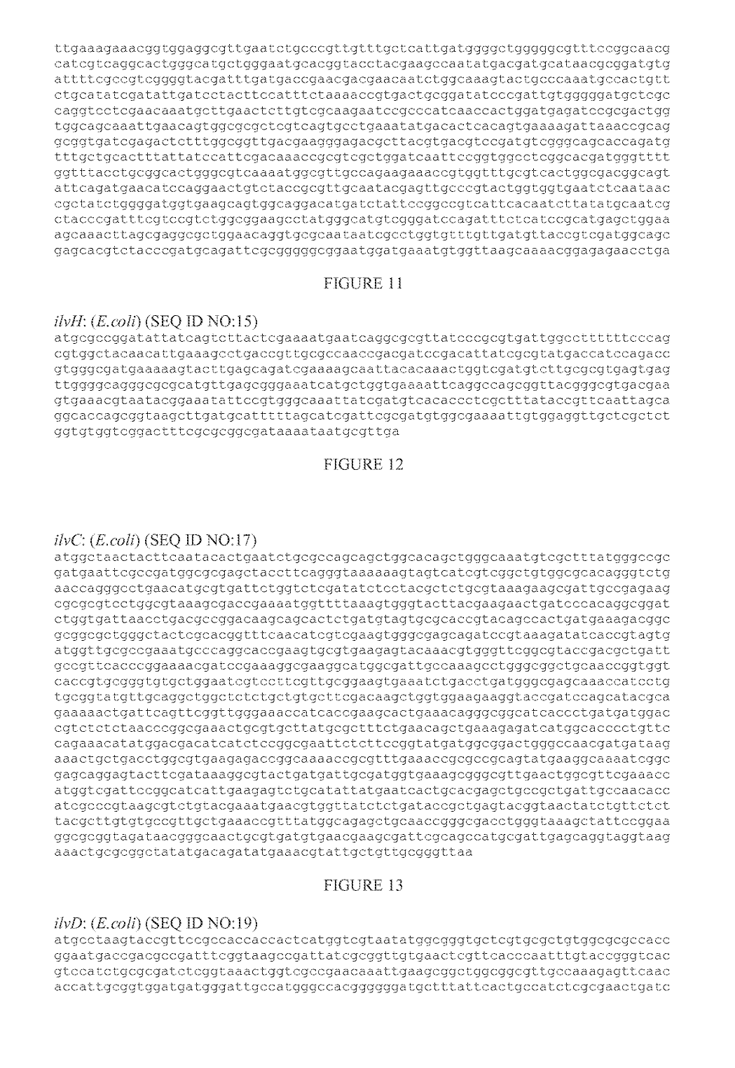

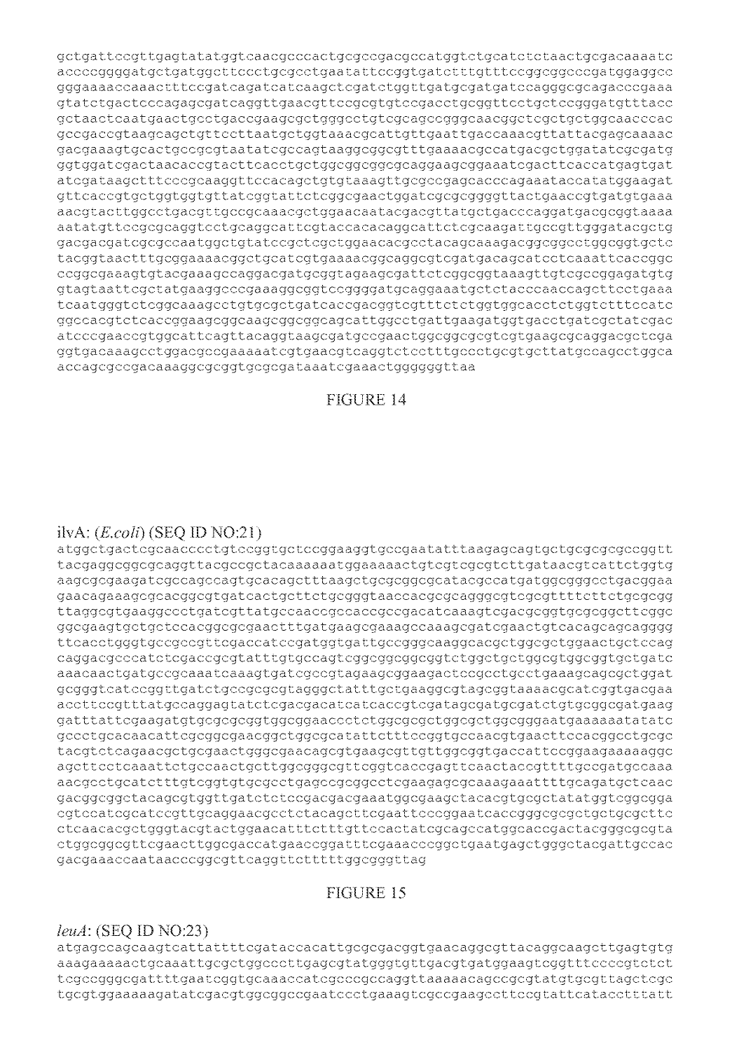

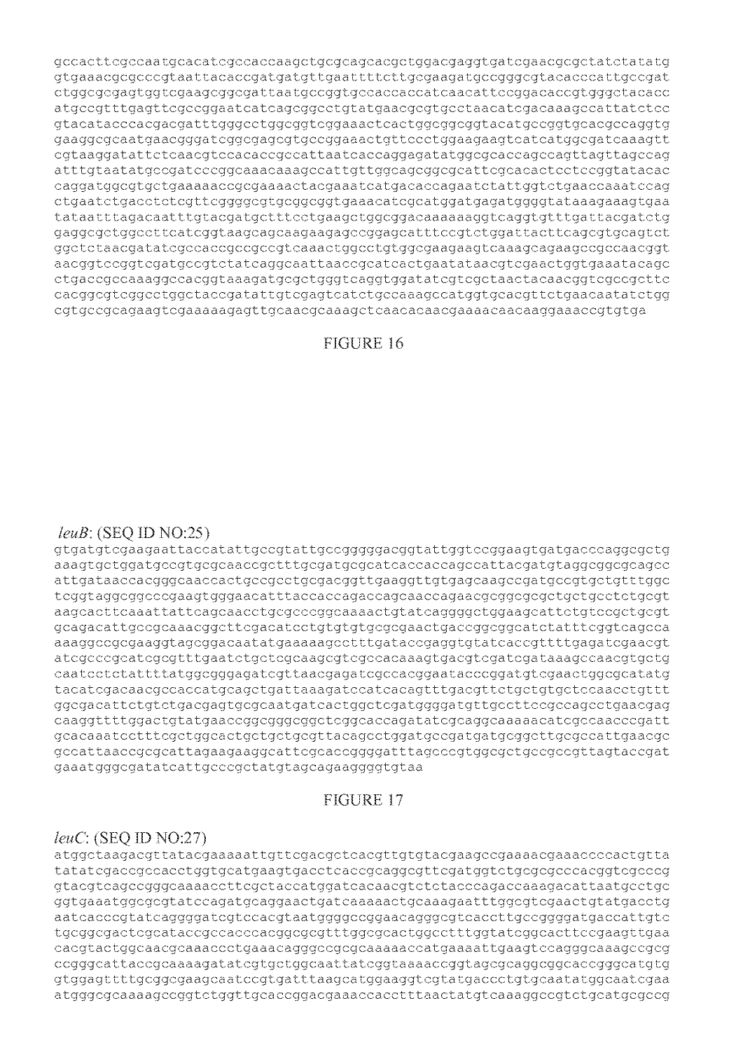

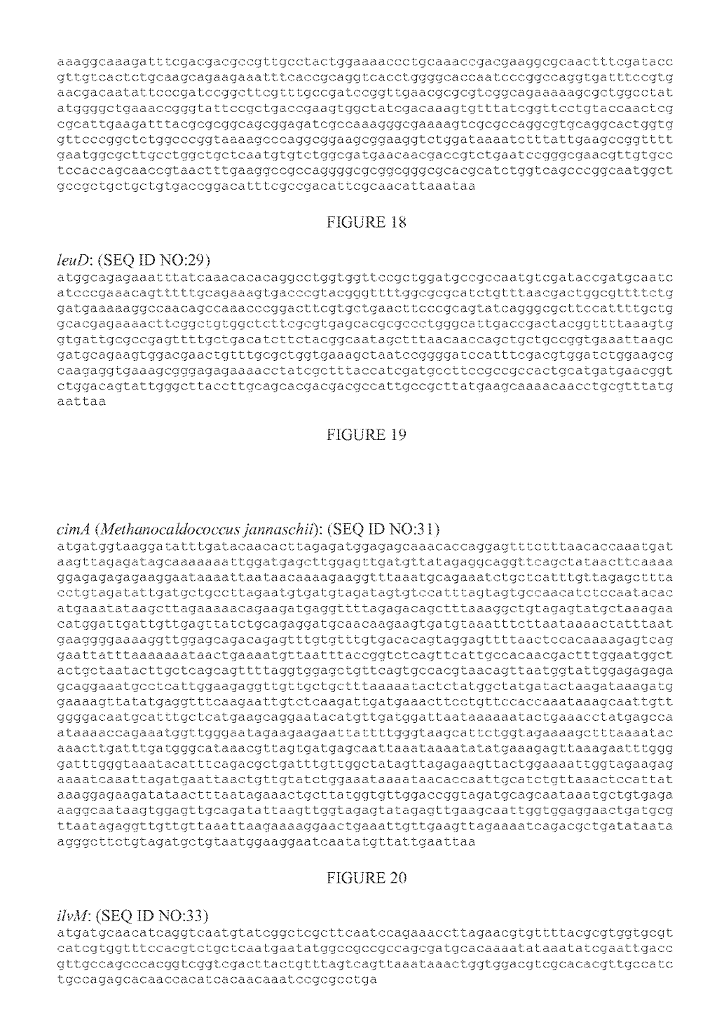

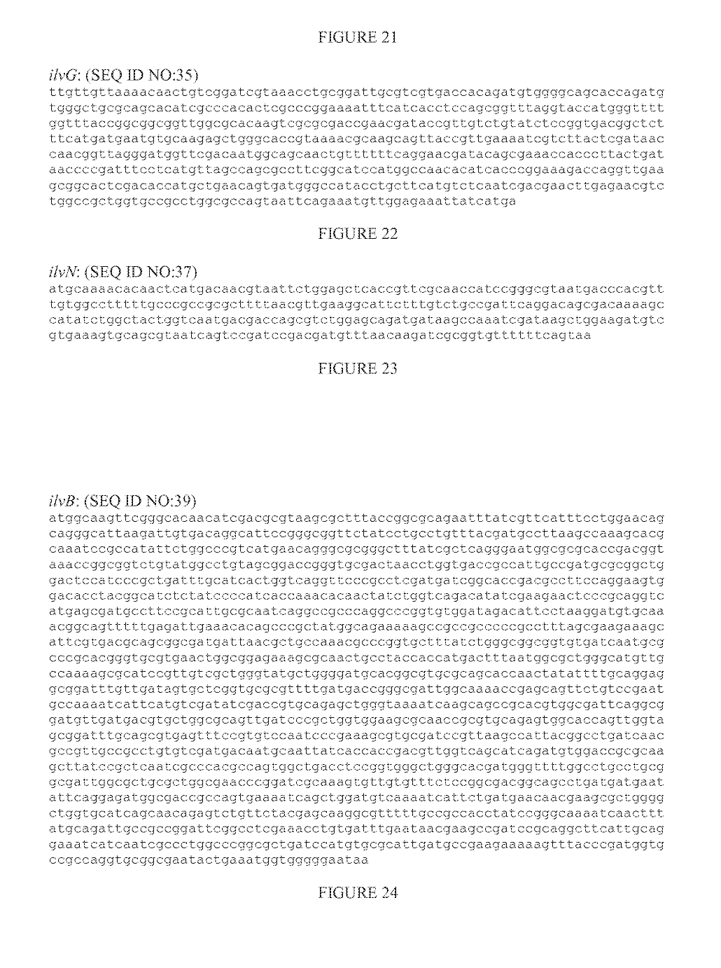

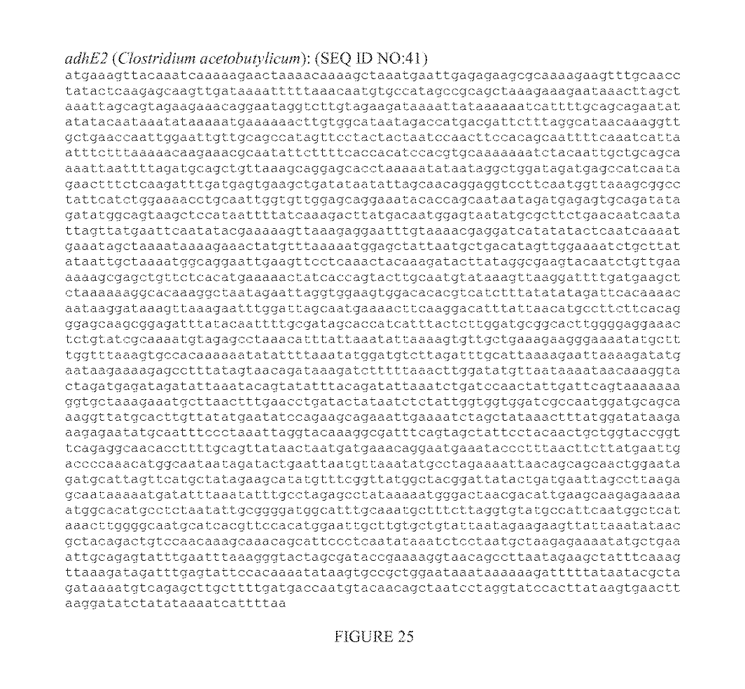

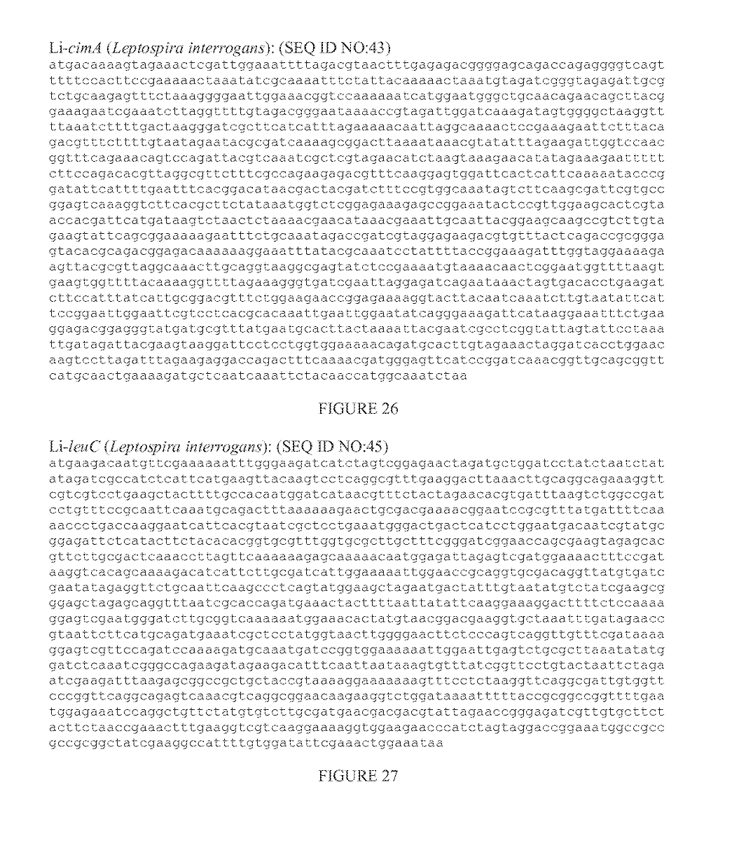

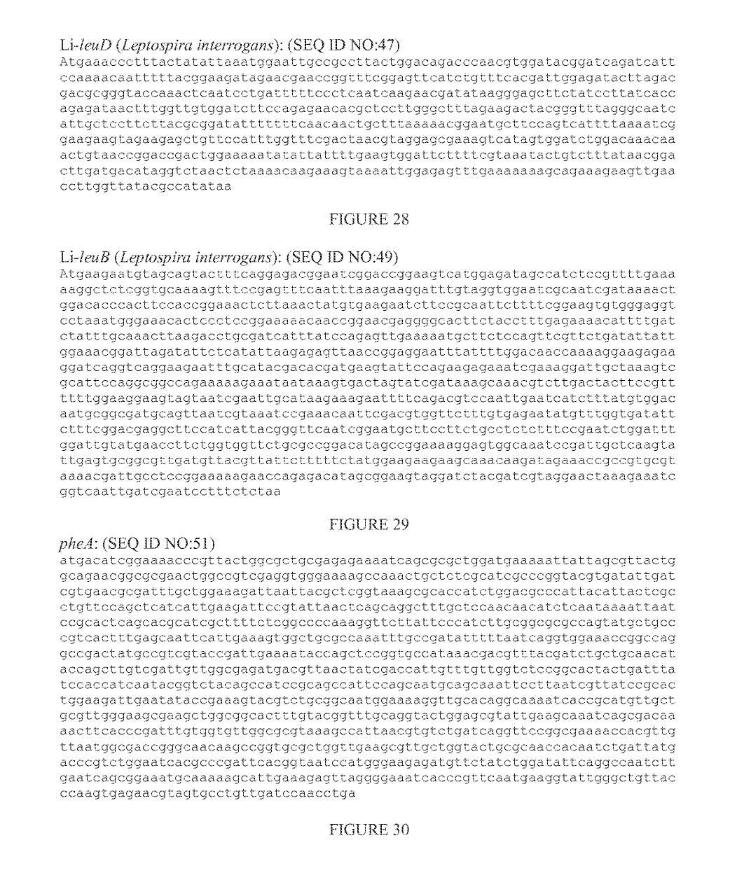

The disclosure also provides a bioreactor and bioreactor system for higher alcohol production. An integrated bioreactor (10) comprises an anode (30), a cathode (40), a container (20) comprising at least one wall and having at least one opening (25), wherein the anode (30) and cathode (40) are disposed within the container (20). A liquid permeable separator (60), surrounds the anode (30) defining an anode space (35), wherein the separator (60) substantially confines free-radicals produced at the anode (30) within the anode space (35). The bioreactor (10) comprises at least one fluid inlet (e.g., 50, 80, 90) extending into the container. As shown in FIG. 4F, a bioreactor of the disclosure (10) comprises a container (20), which is a typical fermentation vat, cell culture container and the like. For example, the container (20) can comprise metal, plastic, glass and the like. The container (20) comprises at least one wall and at least one opening for delivery of cells, electrodes, wires, etc. The container (20) can be of any size, for example, for laboratory research it can be of a size to hold milliliters to liters. For large batch production the container can be of a size to hold tens, hundreds or thousands of liters of media (100).

FIG. 4F also depicts microorganisms (110) which can be any of the recombinant microorganisms described herein for production of a desired alcohol (e.g., isobutanol or 3-methyl-1-butanol). Microorganisms (110) are suspended and cultured in media (100).

The disclosure also depicts an anode (30) and cathode (40). The anode and cathode are arranged to permit, for example, water splitting (H₂O→H₂ and O₂) and/or the production of formate. There are thousands of descriptions of various water splitting systems comprising anodes and cathodes including semiconductive materials, conductive membranes etc. Such systems can be run using solar energy, light energy and electrical energy. Depicted in FIG. 4F is a general schematic showing two electrodes (30 and 40) separated by a porous material layer, selectively permeable membrane or ion exchange membrane (60). As mentioned briefly above, the ion exchange membrane may comprise the anode and cathode embedded in the membrane itself. In such instances the anode portion is directed away from the microorganisms and the cathode portion is directed towards the microorganisms.

Porous material (60) can be any material that prevents or inhibits the passage of free radical oxide species from the anode portion towards the cathode portion comprising the microorganisms. For example, the porous material (60) is liquid permeable but is selectively permeable or torteous such that free radical/oxygen species cannot easily permeate or pass through the porous material (60). The porous material may be a polymer, solid, glass, ceramic and the like.

The bioreactor (10) also comprises at least one inlet for delivery or removal of gaseous or other fluids. For example, off gases from microorganism metabolism can be removed through gas outlet (50). In addition, CO2 can be delivered by CO₂ inlet (80). As described above CO₂ is the carbon source for alcohol production. In addition, air inlet (90) may be used to delivery O₂ or other desirable gases including H₂. A stirrer (70) can be included and may be controlled magnetically or by direct action to maintain suspension of microorganism species.

Accordingly, the disclosure provides a bioreactor and further provides metabolically engineered microorganisms comprising biochemical pathways for the production of higher alcohols including isobutanol, 1-butanol, 1-propanol, 2-methyl-1-butanol, 3-methyl-1-butanol and 2-phenylethanol from a suitable substrate. A metabolically engineered microorganism of the disclosure comprises one or more recombinant polynucleotides within the genome of the organism or external to the genome within the organism to, for example, provide a pathway as set forth in Scheme I. The microorganism can comprise a reduction, disruption or knockout of a gene found in the wild-type organism and/or introduction of a heterologous polynucleotide.

In one embodiment, the disclosure provides a recombinant microorganism comprising elevated expression of at least one target enzyme as compared to a parental microorganism or encodes an enzyme not found in the parental organism. In another or further aspect, the microorganism comprises a reduction, disruption or knockout of at least one gene encoding an enzyme that competes with a metabolite necessary for the production of a desired higher alcohol product. The recombinant microorganism produces at least one metabolite involved in a biosynthetic pathway for the production of an alcohol such as, for example, isobutanol or 3-methyl-1-butanol. In general, the recombinant microorganisms comprises at least one recombinant metabolic pathway that comprises a target enzyme and may further include a reduction in activity or expression of an enzyme in a competitive biosynthetic pathway or to improve metabolic flux down a desired pathway. The pathway acts to modify a substrate or metabolic intermediate in the production of an alcohol such as, for example, isobutanol or 3-methyl-1-butanol. The target enzyme is encoded by, and expressed from, a polynucleotide derived from a suitable biological source. In some embodiments, the polynucleotide comprises a gene derived from a bacterial or yeast source and recombinantly engineered into the microorganism of the disclosure.

In another embodiment a method of producing a recombinant microorganism that converts a suitable carbon substrate to e.g., 1-propanol, isobutanol, 1-butanol, 2-methyl 1-butanol, 3-methyl 1-butanol or 2-phenylethanol is provided. The method includes transforming a microorganism with one or more recombinant polynucleotides encoding polypeptides that include, for example, acetohydroxy acid synthase (e.g., ilvIH operon), acetohydroxy acid isomeroreductase (e.g., ilvC), dihydroxy-acid dehydratase (e.g., ilvD), 2-keto-acid decarboxylase (e.g., PDC6, ARO10, THI3, kivd, or pdc), 2-isopropylmalate synthase (e.g., leuA), beta-isopropylmalate dehydrogenase (e.g., leuB), isopropylmalate isomerase (e.g., leuCD operon), threonine dehydratase (e.g., ilvA), alpha-isopropylmalate synthase (e.g., cimA), beta-isopropylmalate dehydrogenase (e.g., leuB), isopropylmalate isomerase (e.g., leuCD operon), threonine dehydratase (e.g., ilvA), acetolactate synthase (e.g., ilvMG or ilvNB), acetohydroxy acid isomeroreductase (e.g., ilvC), dihydroxy-acid dehydratase (e.g., ilvD), beta-isopropylmalate dehydrogenase (e.g., leuB), chorismate mutase P/prephenate dehydratase (e.g., pheA), chorismate mutase T/prephenate dehydrogenase (e.g., tyrA), 2-keto-acid decarboxylase (e.g., kivd, PDC6, or THI3), and alcohol dehydrogenase activity. Polynucleotides that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a polynucleotide, such as the addition of a non-functional or non-coding sequence, is a conservative variation of the basic nucleic acid. The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

In another embodiment a method for producing e.g., 1-propanol, isobutanol, 1-butanol, 2-methyl 1-butanol, 3-methyl 1-butanol or 2-phenylethanol is provided. The method includes culturing a recombinant microorganism as provided herein in the presence of a suitable substrate and under conditions suitable for the conversion of the substrate to 1-propanol, isobutanol, 1-butanol, 2-methyl 1-butanol, 3-methyl 1-butanol or 2-phenylethanol. The alcohol produced by a microorganism provided herein can be detected by any method known to the skilled artisan. Such methods include mass spectrometry. Culture conditions suitable for the growth and maintenance of a recombinant microorganism provided herein are described in the Examples below. The skilled artisan will recognize that such conditions can be modified to accommodate the requirements of each microorganism.

Appropriate culture conditions are conditions of culture medium pH, ionic strength, nutritive content, etc.; temperature; oxygen/CO₂/nitrogen content; humidity; and other culture conditions that permit production of the compound by the host microorganism, i.e., by the metabolic action of the microorganism. Appropriate culture conditions are well known for microorganisms that can serve as host cells.

As mentioned above, various microorganisms can be manipulated/engineered to produce an alcohol as described herein. It is understood that a range of microorganisms can be modified to include a recombinant metabolic pathway suitable for the production of e.g., 1-propanol, isobutanol, 1-butanol, 2-methyl 1-butanol, 3-methyl 1-butanol or 2-phenylethanol and which couple a “light-reaction” or a “non-light-reaction” that utilize H₂ or formate for producing reducing intermediates in the production of the alcohol. It is also understood that various microorganisms can act as “sources” for genetic material encoding target enzymes suitable for use in a recombinant microorganism provided herein. The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt ((NaCl)); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

Photoautotrophic bacteria are typically Gram-negative rods which obtain their energy from sunlight through the processes of photosynthesis. In this process, sunlight energy is used in the synthesis of carbohydrates, which in recombinant photoautotrophs can be further used as intermediates in the synthesis of biofuels. In other embodiment, the photoautotrophs serve as a source of carbohydrates for use by non-photosynthetic microorganism (e.g., recombinant E. coli) to produce biofuels by a metabolically engineered microorganism. Certain photoautotrophs called anoxygenic photoautotrophs grow only under anaerobic conditions and neither use water as a source of hydrogen nor produce oxygen from photosynthesis. Other photoautotrophic bacteria are oxygenic photoautotrophs. These bacteria are typically cyanobacteria. They use chlorophyll pigments and photosynthesis in photosynthetic processes resembling those in algae and complex plants. During the process, they use water as a source of hydrogen and produce oxygen as a product of photosynthesis.

Cyanobacteria include various types of bacterial rods and cocci, as well as certain filamentous forms. The cells contain thylakoids, which are cytoplasmic, plate like membranes containing chlorophyll. The organisms produce heterocysts, which are specialized cells believed to function in the fixation of nitrogen compounds.

The term “recombinant microorganism” and “recombinant host cell” are used interchangeably herein and refer to microorganisms that have been genetically modified to express or over-express endogenous polynucleotides, or to express non-endogenous sequences, such as those included in a vector, or which have a reduction in expression of an endogenous gene. The polynucleotide generally encodes a target enzyme involved in a metabolic pathway for producing a desired metabolite as described above. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

The term “alcohol” includes for example 1-propanol, isobutanol, 1-butanol, 2-methyl 1-butanol, 3-methyl 1-butanol or 2-phenylethanol. The term “1-butanol” or “n-butanol” generally refers to a straight chain isomer with the alcohol functional group at the terminal carbon. The straight chain isomer with the alcohol at an internal carbon is sec-butanol or 2-butanol. The branched isomer with the alcohol at a terminal carbon is isobutanol, and the branched isomer with the alcohol at the internal carbon is tert-butanol. In one embodiment, the alcohol is isobutanol or 3-methyl-1-butanol.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

As used herein, the term “metabolically engineered” or “metabolic engineering” involves rational pathway design and assembly of biosynthetic genes, genes associated with operons, and control elements of such polynucleotides, for the production of a desired metabolite, such as a 2-keto acid or higher alcohol, in a microorganism. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one aspect, where the polynucleotide is xenogenetic to the host organism, the polynucleotide can be codon optimized.

A “metabolite” refers to any substance produced by metabolism or a substance necessary for or taking part in a particular metabolic process. A metabolite can be an organic compound that is a starting material (e.g., glucose or pyruvate), an intermediate (e.g., 2-keto acid) in, or an end product (e.g., isobutanol, 3-methyl 1-butanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

Exemplary metabolites include pyruvate, isobutanol, 3-methyl 1-butanol and 2-keto acids. As depicted in FIG. 1, exemplary 2-keto acid intermediates include 2-ketoisovalerate and 2-ketoisocaproate. The exemplary 2-keto acids shown in FIG. 1 may be used as metabolic intermediates in the production of isobutanol and 3-methyl 1-butanol. For example, the metabolite 2-ketoisovalerate can be produced by a recombinant microorganism metabolically engineered to express or over-express acetohydroxy acid synthase enzymes encoded by, for example, ilvIHCD genes. This metabolite can then be used in the production of isobutanol or 3-methyl 1-butanol. The metabolite pyruvate can be used to produce 2-ketoisovalerate and 2-ketoisocaproate by a recombinant microorganism.

Recombinant microorganisms provided herein can express a plurality of target enzymes involved in pathways for the production of, for example, isobutanol or 3-methyl 1-butanol from using a suitable carbon substrate. The disclosure can utilize parental organisms with heterologous polynucleotides to promote the biosynthetic pathway for the production of biofuels. In one embodiment, Ralstonia eutropha is used as the parental microorganism for isobutanol or 3-methyl-1-butanol production. In other embodiments, the disclosure provides a recombinant microorganism that comprises a heterologous CO₂ fixation enzyme (e.g., RuBisCo) and one or more enzymes that can convert H₂ or formate to NAD(P)H such as a formate dehydrogenase or a membrane bound hydrogenase or soluble hydrogenase that oxidize H₂ and formate and reduce NAD and/or NADP.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite or the reduction or elimination of the production of an undesired metabolite. In an illustrative embodiment, the introduction of genetic material into a parental microorganism results in a new or modified ability to produce an alcohol such as isobutanol or 3-methyl 1-butanol. The genetic material introduced into the parental microorganism contains gene(s), or parts of genes, coding for one or more of the enzymes involved in a biosynthetic pathway for the production of an alcohol and may also include additional elements for the expression and/or regulation of expression of these genes, e.g. promoter sequences.

An engineered or modified microorganism can also include in the alternative or in addition to the introduction of a genetic material into a host or parental microorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produce a new or greater quantities of an intracellular metabolite, improve the flux of a metabolite down a desired pathway, and/or reduce the production of undesirable by-products).

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any CO₂ or a biomass derived sugar, but also intermediate and end product metabolites used in a pathway associated with a metabolically engineered microorganism as described herein. A “biomass derived sugar” includes, but is not limited to, molecules such as glucose, sucrose, mannose, xylose, and arabinose. The term biomass derived sugar encompasses suitable carbon substrates ordinarily used by microorganisms, such as 6 carbon sugars, including but not limited to glucose, lactose, sorbose, fructose, idose, galactose and mannose all in either D or L form, or a combination of 6 carbon sugars, such as glucose and fructose, and/or 6 carbon sugar acids including, but not limited to, 2-keto-L-gulonic acid, idonic acid (IA), gluconic acid (GA), 6-phosphogluconate, 2-keto-D-gluconic acid (2 KDG), 5-keto-D-gluconic acid, 2-ketogluconatephosphate, 2,5-diketo-L-gulonic acid, 2,3-L-diketogulonic acid, dehydroascorbic acid, erythorbic acid (EA) and D-mannonic acid.

The disclosure demonstrates that the expression of one or more heterologous polynucleotide or over-expression of one or more heterologous polynucleotide encoding a polypeptide having ketoacid decarboxylase and a polypeptide having alcohol dehydrogenase in the presence of a polypeptide having α-isopropylmalate synthase, a polypeptide having β-isopropylmalate dehydrogenase, a polypeptide having α-isopropylmalate isomerase, a polypeptide having threonine dehydratase, a polypeptide having homoserine dehydrogenase activity, a polypeptide having homoserine kinase activity, and a polypeptide having threonine synthase activity to produce isobutanol or 3MB.

For example, the disclosure demonstrates that with over-expression of the heterologous kivd or kdc, adh2, ilvI, IlvH, IlvC, IlvD, leuA, leuB, leuC, and/or leuD (or a Leu operon, e.g., leuABCD), isobutanol and 3-methyl-1-butanol can be produced.

It should be understood that various microorganisms inherently comprise parts of a useful pathway, but not the whole pathway leading to biofuel production. For example, photoautotrophs comprise enzymes that can fix CO₂, but utilize light reactions for generating the necessary reducing metabolites. In such instances it would be unnecessary to engineer the organism to provide an enzyme that fixes CO₂ because the organism inherently does so; however, the organism would be engineered to express enzymes the convert the “fixed” CO₂ in the form of pyruvate to the desired alcohol or to include enzymes to convert H₂ or formate to NAD(P)H.

Accordingly, provided herein are recombinant microorganisms that produce isobutanol and in some aspects may include the elevated expression of target enzymes such as acetohydroxy acid synthase (e.g., ilvIH operon), acetohydroxy acid isomeroreductase (e.g., ilvC), dihydroxy-acid dehydratase (e.g., ilvD), 2-keto-acid decarboxylase (e.g., PDC6, ARO10, THI3, kivd, kdc, or pdc), and alcohol dehydrogenase (e.g., ADH2). The microorganism may further include the deletion or inhibition of expression of an ethanol dehydrogenase (e.g., an adhE), ldh (e.g., an ldhA), frd (e.g., an frdB, an frdC or an frdBC), fnr, leuA, ilvE, poxB, ilvA, pflB, phaA, phaB, phaC, pta gene, or any combination thereof, to increase the availability of pyruvate or reduce enzymes that compete for a metabolite in a desired biosynthetic pathway. In some aspects the recombinant microorganism may include the elevated expression of acetolactate synthase (e.g., alsS), acteohydroxy acid isomeroreductase (e.g., ilvC), dihydroxy-acid dehydratase (e.g., ilvD), 2-keto acid decarboxylase (e.g., PDC6, ARO10, TH13, kivd, kdc, or pdc), and alcohol dehydrogenase (e.g., ADH2). With reference to alcohol dehydrogenases, although ethanol dehydrogenase is an alcohol dehydrogenase, the synthesis of ethanol is undesirable as a by-product in the biosynthetic pathways. Accordingly, reference to an increase in alcohol dehydrogenase activity or expression in a microorganism specifically excludes ethanol dehydrogenase activity.

Also provided are recombinant microorganisms that produce 3-methyl 1-butanol and may include the elevated expression of target enzymes such as acetolactate synthase (e.g., alsS), acetohydroxy acid synthase (e.g., ilvIH), acetolactate synthase (e.g., ilvMG) or (e.g., ilvNB), acetohydroxy acid isomeroreductase (e.g., ilvC), dihydroxy-acid dehydratase (e.g., ilvD), 2-isopropylmalate synthase (leuA), isopropylmalate isomerase (e.g., leuC, D or leuCD operon), beta-isopropylmalate dehydrogenase (e.g., leuB), 2-keto-acid decarboxylase (e.g., kivd, kdc, PDC6, or THI3), and alcohol dehydrogenase (e.g., ADH2).

As previously noted the target enzymes described throughout this disclosure generally produce metabolites. For example, threonine dehydratase can be encoded by a polynucleotide derived from an ilvA gene. Acetohydroxy acid synthase can be encoded by a polynucleotide derived from an ilvIH operon. Acetohydroxy acid isomeroreductase can be encoded by a polynucleotide derived from an ilvC gene. Dihydroxy-acid dehydratase can be encoded by a polynucleotide derived from an ilvD gene. 2-keto-acid decarboxylase can be encoded by a polynucleotide derived from a PDC6, ARO10, THI3, kivd, kdc, and/or pdc gene. Alcohol dehydrogenase can be encoded by a polynucleotide derived from an ADH2 gene. Additional enzymes and exemplary genes are described throughout this document. Homologs of the various polypeptides and polynucleotides can be derived from any biologic source that provides a suitable polynucleotide encoding a suitable enzyme. Homologs, for example, can be identified by reference to various databases.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a function enzyme activity using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

It is understood that polynucleotides include “genes” and that nucleic acid molecules include “vectors” or “plasmids.” For example, a polynucleotide encoding a keto thiolase can be encoded by an atoB gene or homolog thereof, or a fadA gene or homolog thereof. Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence. The term “nucleic acid” or “recombinant nucleic acid” refers to polynucleotides such as deoxyribonucleic acid (DNA), and, where appropriate, ribonucleic acid (RNA). The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, reference to a kivd gene includes homologs (e.g., pdc6, aro10, thI3, pdc, kdcA, pdc1, pdc5) from other organisms encoding an enzyme having substantially similar enzymatic activity, as well as genes having at least 30, 40, 50, 60, 70, 80, 85, 90, 95, 98, or 99% identity to the referenced gene and which encodes an enzyme having substantially similar enzymatic activity as the referenced gene. For example, pyruvate decarboxylase of Kluyveromyces lactis has 37% identity to Kivd at the amino acids level; kivd and thI3 are 32% identical at the nucleic acid level; Alcohol dehydrogenase of Schizosaccharomyces pombe has 52% identity to ADH2 of Saccharomyces cerevisiae at the amino acid sequence level; S. cerevisiae adh2 and Lactococcus Lactis adh are 49% identical; KIVD (Lactococcus lactis) and PDC6 (Saccharomyces cerevisiae) share 36% identity (Positives=322/562 (57%), Gaps=24/562 (4%)); KIVD (Lactococcus lactis and THI3 (Saccharomyces cerevisiae) share 32% identity (Positives=307/571 (53%), Gaps=35/571 (6%)); kivd (Lactococcus lactis) and ARO10 (Saccharomyces cerevisiae) share 30% identikit (Positives=296/598 (49%), Gaps=65/598 (10%)); ARO10 (Saccharomyces cerevisiae) and PDC6 (Saccharomyces cerevisiae) share 34% identity (Positives=320/616 (51%), Gaps=61/616 (9%)); ARO10 (Saccharomyces cerevisiae) and THI3 (Saccharomyces cerevisiae) share 30% identity (Positives=304/599 (50%), Gaps=48/599 (8%)); ARO10 (Saccharomyces cerevisiae) and Pyruvate decarboxylase (Clostridium acetobutylicum ATCC 824) share 30% identity (Positives=291/613 (47%), Gaps=73/613 (11%)); PDC6 ((Saccharomyces cerevisiae) and THI3 (Saccharomyces cerevisiae) share 50% identikit (Positives=402/561 (71%), Gaps=17/561 (3%)); PDC6 (Saccharomyces cerevisiae) and Pyruvate decarboxylase (Clostridium acetobutylicum ATCC 824) share 38% identity (Positives=328/570 (57%), Gaps=30/570 (5%)); and THI3 (Saccharomyces cerevisiae) and Pyruvate decarboxylase (Clostridium acetobutylicum ATCC 824) share 35% identity (Positives=284/521 (54%), Gaps=25/521 (4%)). Sequence for each of the genes and polypeptides/enzymes listed herein can be readily identified using databases available on the World-Wide-Web (see, e.g., http:(//)eecoli.kaist.ac.krmain.html). In addition, the amino acid sequence and nucleic acid sequence can be readily compared for identity using commonly used algorithms in the art.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

The term “operon” refers two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that has been genetically modified but which does not express or over-express a target enzyme e.g., an enzyme involved in the biosynthetic pathway for the production of a desired metabolite such as 1-propanol, isobutanol, 1-butanol, 2-methyl 1-butanol, 3-methyl 1-butanol or 2-phenylethanol. For example, a wild-type microorganism can be genetically modified to express or over express a first target enzyme such as thiolase. This microorganism can act as a parental microorganism in the generation of a microorganism modified to express or over-express a second target enzyme e.g., hydroxybutyryl CoA dehydrogenase. In turn, the microorganism modified to express or over express e.g., thiolase and hydroxybutyryl CoA dehydrogenase can be modified to express or over express a third target enzyme e.g., crotonase. Accordingly, a parental microorganism functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme in to a parental microorganism.

A “protein” or “polypeptide”, which terms are used interchangeably herein, comprises one or more chains of chemical building blocks called amino acids that are linked together by chemical bonds called peptide bonds. An “enzyme” means any substance, composed wholly or largely of protein, that catalyzes or promotes, more or less specifically, one or more chemical or biochemical reactions. The term “enzyme” can also refer to a catalytic polynucleotide (e.g., RNA or DNA). A “native” or “wild-type” protein, enzyme, polynucleotide, gene, or cell, means a protein, enzyme, polynucleotide, gene, or cell that occurs in nature.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

Those of skill in the art will recognize that, due to the degenerate nature of the genetic code, a variety of DNA compounds differing in their nucleotide sequences can be used to encode a given amino acid sequence of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate embodiments of the disclosure.

The disclosure provides nucleic acid molecules in the form of recombinant DNA expression vectors or plasmids, as described in more detail below, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

Provided herein are methods for the heterologous expression of one or more of the biosynthetic genes involved in 1-propanol, isobutanol, 1-butanol, 2-methyl 1-butanol, 3-methyl 1-butanol, and/or 2-phenylethanol biosynthesis and recombinant DNA expression vectors useful in the method. Thus, included within the scope of the disclosure are recombinant expression vectors that include such nucleic acids. The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (tip), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of PKS and/or other biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The following table and the disclosure provide non-limiting examples of genes and homologs for each gene having polynucleotide and polypeptide sequences available to the skilled person in the art.

TABLE 3
Depicts recombinant pathways for the production of various higher alcohols (″+″ =
expression, increase expression or activity/″−″ = reduced expression or activity or knockout*).

| | | | | | | | 3-M-1- | 2-M-1- |
|---|---|---|---|---|---|---|---|---|
| | | | 1-butanol | 1-butanol | 1-propanol | 1-propanol | butanol | butanol |
| | Exemplary | | (via L- | (via | (via L- | (via | (via | (via L- |
| Enzyme | Gene(s) | isobutanol | threonine) | pyruvate) | threonine) | pyruvate) | pyruvate) | threonine) |
| Ethanol | adhE | − | − | − | − | − | − | − |
| Dehydrogenase | | | | | | | | |
| Lactate | ldhA | − | − | − | − | − | − | − |
| Dehydrogenase | | | | | | | | |
| Fumarate reductase | frdBC | − | | | | | − | − |
| | fnr | − | | | | | − | − |
| acetate kinase | ackA | − | − | − | − | − | − | − |
| Phosphate | pta | − | − | − | − | − | − | − |
| acetyltransferase | | | | | | | | |
| Formate | pflB | − | | | | | − | − |
| acetyltransferase | | | | | | | | |
| α-isopropylmalate | leuA | − | + | + | | | + | |
| synthase | | | | | | | | |
| β-isopropylmalate | leuB | | + | + | − | + | + | |
| dehydrogenase, | | | | | | | | |
| α-isopropylmalate | leuC | | + | + | | + | + | |
| isomerase | | | | | | | | |
| α-isopropylmalate | leuD | | + | + | | + | | |
| isomerase | | | | | | | | |
| BCAA | ilvE | − | | | | | − | |
| aminotransferase | | | | | | | | |
| tyrosine | tyrB, | | | | | | − | |
| aminotransferase | tyrAT | | | | | | | |
| pyruvate | poxB | − | − | − | − | − | | |
| dehydrogenase | | | | | | | | |
| acetolactate synthase | ilvB | | − | − | − | − | | |
| acetolactate synthase | ilvI, aisS | | − | − | − | − | | |
| threonine | ilvA, tdcB | − | + | + | + | + | | + |
| dehydratase | | | | | | | | |
| homoserine | metA | | − | − | − | − | | − |
| transsuccinylase | | | | | | | | |
| L-threonine 3- | tdh | | − | − | − | − | | − |
| dehydrogenase | | | | | | | | |
| acetohydroxy acid | ilvHI, | + | | | | | + | + |
| synthase | ilvNB, | | | | | | | |
| | ilvGM, | | | | | | | |
| | alsS | | | | | | | |
| acetohydroxy acid | ilvC, ilv5 | + | | | | | + | + |
| isomeroredutase | | | | | | | | |
| dihydroxy-acid | ilvD, ilv3 | + | | | | | + | + |
| dehydrates | | | | | | | | |
| 2-ketoacid | pdc6, | + | + | + | + | + | + | + |
| decarboxylase | aro10, | | | | | | | |
| | thI3, kivd, | | | | | | | |
| | pdc, kdcA, | | | | | | | |
| | pdc1, pdc5 | | | | | | | |
| alcohol | adh1, | + | + | + | + | + | + | + |
| dehydrogenase | adh2, | | | | | | | |
| | adh3, | | | | | | | |
| | adh4, | | | | | | | |
| | adh5, | | | | | | | |
| | adh6, sfa1 | | | | | | | |
| citramalate synthase | cimA | | | + | | + | | |

*knockout or a reduction in expression are optional in the synthesis of the product, however, such knockouts increase various substrate intermediates and improve yield.

Tables 4-5 set forth reaction pathways for various recombinant microorganism of the disclosure including a list of exemplary genes and homologs and organism source.

TABLE 4
Isobutanol production pathway (via pyruvate)

Reaction 1
pyruvate -> 2-acetolactate
Genes
ilvHI(E. coli), ilvNB(E. coli), ilvGM(E. coli), alsS(Bacillus subtilis) or
homologs thereof
Reaction 2
2-acetolactate -> 2,3-dihydroxy-isovalerate
Genes
ilvC(E. coli) or homologs thereof
Reaction 3
2,3-dihydroxy-isovalerate -> 2-keto-isovalerate
Genes
ilvD(E. coli) or homologs thereof
Reaction 4
2-keto-isovalerate -> isobutaylaldehyde
Genes
kivd(Lactococcus lactis), kdcA(Lactococcus lactis), PDC1(Saccharomyces
cerevisiae), PDC5(Saccharomyces cerevisiae), PDC6(Saccharomyces
cerevisiae) THI3(Saccharomyces cerevisiae), ARO10(Saccharomyces
cerevisiae) or homologs thereof
Reaction 5
isobutryraldehyde -> isobutanol
Genes
ADH1(Saccharomyces cerevisiae), ADH2(Saccharomyces cerevisiae),
ADH3(Saccharomyces cerevisiae), ADH4(Saccharomyces cerevisiae),
ADH5(Saccharomyces cerevisiae), ADH6(Saccharomyces cerevisiae),
SFA1(Saccharomyces cerevisiae) or homologs thereof

TABLE 5
3-methyl-1-butanol production pathway (via pyruvate)

Reaction 1
pyruvate -> 2-acetolactate
Gene
ilvHI(E. coli), ilvNB(E. coli), ilvGM(E. coli), alsS(Bacillus subtilis) or
homologs thereof
Reaction 2
2-acetolactate -> 2,3-dihydroxy-isovalerate
Genes
ilvC(E. coli) or homologs thereof
Reaction 3
2,3-dihydroxy-isovalerate -> 2-keto-isovalerate
Genes
ilvD(E. coli) or homologs thereof
Reaction 4
2-keto-isovalerate -> 2-isopropylmalate
Genes
leuA(E. coli) or homologs thereof
Reaction 5
2-isopropylmalate -> 3-isopropylmalate
Genes
leuCD(E. coli) or homologs thereof
Reaction 6
3-isopropylmalate -> 2-isopropyl-3-oxosuccinate
Genes
leuB(E. coli) or homologs thereof
Reaction 7
2-isopropyl-3-oxosuccinate -> 2-ketoisocaproate
Gene
(spontaneous)
Reaction 8
2-ketoisocaproate -> 3-methylbutyraldehyde
Genes
kivd(Lactococcus lactis), kdcA(Lactococcus lactis), PDC1(Saccharomyces
cerevisiae), PDC5(Saccharomyces cerevisiae), PDC6(Saccharomyces
cerevisiae) THI3 (Saccharomyces cerevisiae), ARO10(Saccharomyces
cerevisiae) or homologs thereof
Reaction 9
3-methylbutyraldehyde -> 3-methyl-1-butanol
Genes
ADH1(Saccharomyces cerevisiae), ADH2(Saccharomyces cerevisiae),
ADH3(Saccharomyces cerevisiae), ADH4(Saccharomyces cerevisiae),
ADH5(Saccharomyces cerevisiae), ADH6(Saccharomyces cerevisiae),
SFA1(Saccharomyces cerevisiae) or homologs thereof

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

Ethanol Dehydrogenase (also referred to as Aldehyde-alcohol dehydrogenase) is encoded in E. coli by adhE. adhE comprises three activities: alcohol dehydrogenase (ADH); acetaldehydeacetyl-CoA dehydrogenase (ACDH); pyruvate-formate-lyase deactivase (PFL deactivase); PFL deactivase activity catalyzes the quenching of the pyruvate-formate-lyase catalyst in an iron, NAD, and CoA dependent reaction. Homologs are known in the art (see, e.g., aldehyde-alcohol dehydrogenase (Polytomella sp. Pringsheim 198.80) gi|40644910|emb|CAD42653.2|(40644910); aldehyde-alcohol dehydrogenase (Clostridium botulinum A str. ATCC 3502) gi|148378348|ref|YP_{—}001252889.1|(148378348); aldehyde-alcohol dehydrogenase (Yersinia pestis CO92) gi|16122410|ref|NP_{—}405723.1|(16122410); aldehyde-alcohol dehydrogenase (Yersinia pseudotuberculosis IP 32953) gi|51596429|ref|YP_{—}070620.1|(51596429); aldehyde-alcohol dehydrogenase (Yersinia pestis CO92) gi|115347889|emb|CAL20810.1|(115347889); aldehyde-alcohol dehydrogenase (Yersinia pseudotuberculosis IP 32953) gi|51589711|emb|CAH21341.1|(51589711); Aldehyde-alcohol dehydrogenase (Escherichia coli CFT073) gi|26107972|gb|AAN80172.1|AE016760_{—}31 (26107972); aldehyde-alcohol dehydrogenase (Yersinia pestis biovar Microtus str. 91001) gi|45441777|ref|NP_{—}993316.1|(45441777); aldehyde-alcohol dehydrogenase (Yersinia pestis biovar Microtus str. 91001) gi|45436639|gb|AAS62193.1|(45436639); aldehyde-alcohol dehydrogenase (Clostridium perfringens ATCC 13124) gi|110798574|ref|YP_{—}697219.1|(110798574); aldehyde-alcohol dehydrogenase (Shewanella oneidensis MR-1)gi|24373696|ref|NP_{—}717739.1|(24373696); aldehyde-alcohol dehydrogenase (Clostridium botulinum A str. ATCC 19397) gi|153932445|ref|YP_{—}001382747.1|(153932445); aldehyde-alcohol dehydrogenase (Yersinia pestis biovar Antigua str. E1979001) gi|165991833|gb|EDR44134.1|(165991833); aldehyde-alcohol dehydrogenase (Clostridium botulinum A str. Hall) gi|153937530|ref|YP_{—}001386298.1|(153937530); aldehyde-alcohol dehydrogenase (Clostridium perfringens ATCC 13124) gi|110673221|gb|ABG82208.1|(110673221); aldehyde-alcohol dehydrogenase (Clostridium botulinum A str. Hall) gi|152933444|gb|ABS38943.1|(152933444); aldehyde-alcohol dehydrogenase (Yersinia pestis biovar Orientalis str. F1991016) gi|165920640|gb|EDR37888.1|(165920640); aldehyde-alcohol dehydrogenase (Yersinia pestis biovar Orientalis str. IP275)gi|165913933|gb|EDR32551.1|(165913933); aldehyde-alcohol dehydrogenase (Yersinia pestis Angola) gi|162419116|ref|YP_{—}001606617.1|(162419116); aldehyde-alcohol dehydrogenase (Clostridium botulinum F str. Langeland) gi|153940830|ref|YP_{—}001389712.1|(153940830); aldehyde-alcohol dehydrogenase (Escherichia coli HS) gi|157160746|ref|YP_{—}001458064.1|(157160746); aldehyde-alcohol dehydrogenase (Escherichia coli E24377A) gi|157155679|ref|YP_{—}001462491.1|(157155679); aldehyde-alcohol dehydrogenase (Yersinia enterocolitica subsp. enterocolitica 8081) gi|123442494|ref|YP_{—}001006472.1|(123442494); aldehyde-alcohol dehydrogenase (Synechococcus sp. JA-3-3Ab) gi|86605191|ref|YP_{—}473954.1|(86605191); aldehyde-alcohol dehydrogenase (Listeria monocytogenes str. 4b F2365) gi|46907864|ref|YP_{—}014253.1|(46907864); aldehyde-alcohol dehydrogenase (Enterococcus faecalis V583) gi|29375484|ref|NP_{—}814638.1|(29375484); aldehyde-alcohol dehydrogenase (Streptococcus agalactiae 2603V/R) gi|22536238|ref|NP_{—}687089.1|(22536238); aldehyde-alcohol dehydrogenase (Clostridium botulinum A str. ATCC 19397) gi|152928489|gb|ABS33989.1|(152928489); aldehyde-alcohol dehydrogenase (Escherichia coli E24377A) gi|157077709|gb|ABV17417.1|(157077709); aldehyde-alcohol dehydrogenase (Escherichia coli HS) gi|157066426|gb|ABV05681.1|(157066426); aldehyde-alcohol dehydrogenase (Clostridium botulinum F str. Langeland) gi|152936726|gb|ABS42224.1|(152936726); aldehyde-alcohol dehydrogenase (Yersinia pestis CA88-4125) gi|149292312|gb|EDM42386.1|(149292312); aldehyde-alcohol dehydrogenase (Yersinia enterocolitica subsp. enterocolitica 8081) gi|122089455|emb|CAL12303.1|(122089455); aldehyde-alcohol dehydrogenase (Chlamydomonas reinhardtii) gi|92084840|emb|CAF04128.1|(92084840); aldehyde-alcohol dehydrogenase (Synechococcus sp. JA-3-3Ab) gi|86553733|gb|ABC98691.1|(86553733); aldehyde-alcohol dehydrogenase (Shewanella oneidensis MR-1) gi|24348056|gb|AAN55183.1|AE015655_{—}9(24348056); aldehyde-alcohol dehydrogenase (Enterococcus faecalis V583) gi|29342944|gb|AAO80708.1|(29342944); aldehyde-alcohol dehydrogenase (Listeria monocytogenes str. 4b F2365) gi|46881133|gb|AAT04430.1|(46881133); aldehyde-alcohol dehydrogenase (Listeria monocytogenes str. 1/2a F6854) gi|47097587|ref|ZP_{—}00235115.1|(47097587); aldehyde-alcohol dehydrogenase (Listeria monocytogenes str. 4b H7858) gi|47094265|ref|ZP_{—}00231973.1|(47094265); aldehyde-alcohol dehydrogenase (Listeria monocytogenes str. 4b H7858) gi|47017355|gb|EAL08180.1|(47017355); aldehyde-alcohol dehydrogenase (Listeria monocytogenes str. 1/2a F6854) gi|47014034|gb|EAL05039.1|(47014034); aldehyde-alcohol dehydrogenase (Streptococcus agalactiae 2603V/R) gi|22533058|gb|AAM98961.1|AE014194_{—}6(22533058)p; aldehyde-alcohol dehydrogenase (Yersinia pestis biovar Antigua str. E1979001) gi|166009278|ref|ZP_{—}02230176.1|(166009278); aldehyde-alcohol dehydrogenase (Yersinia pestis biovar Orientalis str. IP275) gi|165938272|ref|ZP_{—}02226831.1|(165938272); aldehyde-alcohol dehydrogenase (Yersinia pestis biovar Orientalis str. F1991016) gi|165927374|ref|ZP_{—}02223206.1|(165927374); aldehyde-alcohol dehydrogenase (Yersinia pestis Angola) gi|162351931|gb|ABX85879.1|(162351931); aldehyde-alcohol dehydrogenase (Yersinia pseudotuberculosis IP 31758) gi|153949366|ref|YP_{—}001400938.1|(153949366); aldehyde-alcohol dehydrogenase (Yersinia pseudotuberculosis IP 31758) gi|152960861|gb|ABS48322.1|(152960861); aldehyde-alcohol dehydrogenase (Yersinia pestis CA88-4125) gi|149365899|ref|ZP_{—}01887934.1|(149365899); Acetaldehyde dehydrogenase (acetylating) (Escherichia coli CFT073) gi|26247570|ref|NP_{—}753610.1|(26247570); aldehyde-alcohol dehydrogenase (includes: alcohol dehydrogenase; acetaldehyde dehydrogenase (acetylating) (EC 1.2.1.10) (acdh); pyruvate-formate-lyase deactivase (pfl deactivase)) (Clostridium botulinum A str. ATCC 3502) gi|148287832|emb|CAL81898.1|(148287832); aldehyde-alcohol dehydrogenase (Includes: Alcohol dehydrogenase (ADH); Acetaldehyde dehydrogenase (acetylating) (ACDH); Pyruvate-formate-lyase deactivase (PFL deactivase)) gi|71152980|sp|P0A9Q7.2|ADHE_{—}ECOLI(71152980); aldehyde-alcohol dehydrogenase (includes: alcohol dehydrogenase and acetaldehyde dehydrogenase, and pyruvate-formate-lyase deactivase (Erwinia carotovora subsp. atroseptica SCRI1043) gi|50121254|ref|YP_{—}050421.1|(50121254); aldehyde-alcohol dehydrogenase (includes: alcohol dehydrogenase and acetaldehyde dehydrogenase, and pyruvate-formate-lyase deactivase (Erwinia carotovora subsp. atroseptica SCRI1043) gi|49611780|emb|CAG75229.1|(49611780); Aldehyde-alcohol dehydrogenase (Includes: Alcohol dehydrogenase (ADH); Acetaldehyde dehydrogenase (acetylating) (ACDH)) gi|19858620|sp|P33744.3|ADHE_CLOAB(19858620); Aldehyde-alcohol dehydrogenase (Includes: Alcohol dehydrogenase (ADH); Acetaldehyde dehydrogenase (acetylating) (ACDH); Pyruvate-formate-lyase deactivase (PFL deactivase)) gi|71152683|sp|P0A9Q8.2|ADHE_ECO57(71152683); aldehyde-alcohol dehydrogenase (includes: alcohol dehydrogenase; acetaldehyde dehydrogenase (acetylating); pyruvate-formate-lyase deactivase (Clostridium difficile 630) gi|126697906|ref|YP_{—}001086803.1|(126697906); aldehyde-alcohol dehydrogenase (includes: alcohol dehydrogenase; acetaldehyde dehydrogenase (acetylating); pyruvate-formate-lyase deactivase (Clostridium difficile 630) gi|115249343|emb|CAJ67156.1|(115249343); Aldehyde-alcohol dehydrogenase (includes: alcohol dehydrogenase (ADH) and acetaldehyde dehydrogenase (acetylating) (ACDH); pyruvate-formate-lyase deactivase (PFL deactivase)) (Photorhabdus luminescens subsp. laumondii TTO1) gi|37526388|ref|NP_{—}929732.1|(37526388); aldehyde-alcohol dehydrogenase 2 (includes: alcohol dehydrogenase; acetaldehyde dehydrogenase) (Streptococcus pyogenes str. Manfredo) gi|134271169|emb|CAM29381.1|(134271169); Aldehyde-alcohol dehydrogenase (includes: alcohol dehydrogenase (ADH) and acetaldehyde dehydrogenase (acetylating) (ACDH); pyruvate-formate-lyase deactivase (PFL deactivase)) (Photorhabdus luminescens subsp. laumondii TTO1) gi|36785819|emb|CAE14870.1|(36785819); aldehyde-alcohol dehydrogenase (includes: alcohol dehydrogenase and pyruvate-formate-lyase deactivase (Clostridium difficile 630) gi|126700586|ref|YP_{—}001089483.1|(126700586); aldehyde-alcohol dehydrogenase (includes: alcohol dehydrogenase and pyruvate-formate-lyase deactivase (Clostridium difficile 630) gi|115252023|emb|CAJ69859.1|(115252023); aldehyde-alcohol dehydrogenase 2 (Streptococcus pyogenes str. Manfredo) gi|139472923|ref|YP_{—}001127638.1|(139472923); aldehyde-alcohol dehydrogenase E (Clostridium perfringens str. 13) gi|18311513|ref|NP_{—}563447.1|(18311513); aldehyde-alcohol dehydrogenase E (Clostridium perfringens str. 13) gi|18146197|dbj|BAB82237.1|(18146197); Aldehyde-alcohol dehydrogenase, ADHE1 (Clostridium acetobutylicum ATCC 824) gi|15004739|ref|NP_{—}149199.1|(15004739); Aldehyde-alcohol dehydrogenase, ADHE1 (Clostridium acetobutylicum ATCC 824) gi|14994351|gb|AAK76781.1|AE001438_{—}34(14994351); Aldehyde-alcohol dehydrogenase 2 (Includes: Alcohol dehydrogenase (ADH); acetaldehydeacetyl-CoA dehydrogenase (ACDH)) gi|2492737|sp|Q24803.1|ADH2_ENTHI(2492737); alcohol dehydrogenase (Salmonella enterica subsp. enterica serovar Typhi str. CT18) gi|16760134|ref|NP_{—}455751.1|(16760134); and alcohol dehydrogenase (Salmonella enterica subsp. enterica serovar Typhi) gi|16502428|emb|CAD08384.1|(16502428)), each sequence associated with the accession number is incorporated herein by reference in its entirety.

Lactate Dehydrogenase (also referred to as D-lactate dehydrogenase and fermentive dehydrognase) is encoded in E. coli by ldhA and catalyzes the NADH-dependent conversion of pyruvate to D-lactate. Because this enzyme competes with metabolites needed for alcohol production, this enzymes activity is typically reduced or knocked out. ldhA homologs and variants are known. In fact there are currently 1664 bacterial lactate dehydrogenases available through NCBI. For example, such homologs and variants include, for example, D-lactate dehydrogenase (D-LDH) (Fermentative lactate dehydrogenase) gi|1730102|sp|P52643.1|LDHD_{—}ECOLI(1730102); D-lactate dehydrogenase gi|1049265|gb|AAB51772.1|(1049265); D-lactate dehydrogenase (Escherichia coli APEC 01) gi|117623655|ref|YP_{—}852568.1|(117623655); D-lactate dehydrogenase (Escherichia coli CFT073) gi|26247689|ref|NP_{—}753729.1|(26247689); D-lactate dehydrogenase (Escherichia coli O157:H7 EDL933) gi|15801748|ref|NP_{—}287766.1|(15801748); D-lactate dehydrogenase (Escherichia coli APEC O1) gi|115512779|gb|ABJ00854.1|(115512779); D-lactate dehydrogenase (Escherichia coli CFT073) gi|26108091|gb|AAN80291.1|AE016760_{—}150(26108091); fermentative D-lactate dehydrogenase, NAD-dependent (Escherichia coli K12) gi|16129341|ref|NP_{—}415898.1|(16129341); fermentative D-lactate dehydrogenase, NAD-dependent (Escherichia coli UTI89) gi|91210646|ref|YP_{—}540632.1|(91210646); fermentative D-lactate dehydrogenase, NAD-dependent (Escherichia coli K12) gi|1787645|gb|AAC74462.1|(1787645); fermentative D-lactate dehydrogenase, NAD-dependent (Escherichia coli W3110) gi|89108227|ref|AP_{—}002007.1|(89108227); fermentative D-lactate dehydrogenase, NAD-dependent (Escherichia coli W3110) gi|1742259|dbj|BAA14990.1|(1742259); fermentative D-lactate dehydrogenase, NAD-dependent (Escherichia coli UTI89) gi|91072220|gb|ABE07101.1|(91072220); fermentative D-lactate dehydrogenase, NAD-dependent (Escherichia coli O157:H7 EDL933) gi|12515320|gb|AAG56380.1|AE005366_{—}6(12515320); fermentative D-lactate dehydrogenase (Escherichia coli O157:H7 str. Sakai) gi|13361468|dbj|BAB35425.1|(13361468); COG1052: Lactate dehydrogenase and related dehydrogenases (Escherichia coli 101-1) gi|83588593|ref|ZP_{—}00927217.1|(83588593); COG1052: Lactate dehydrogenase and related dehydrogenases (Escherichia coli 53638) gi|75515985|ref|ZP_{—}00738103.1|(75515985); COG1052: Lactate dehydrogenase and related dehydrogenases (Escherichia coli E22) gi|75260157|ref|ZP_{—}00731425.1|(75260157); COG1052: Lactate dehydrogenase and related dehydrogenases (Escherichia coli F11) gi|75242656|ref|ZP_{—}00726400.1|(75242656); COG1052: Lactate dehydrogenase and related dehydrogenases (Escherichia coli E110019) gi|75237491|ref|ZP_{—}00721524.1|(75237491); COG1052: Lactate dehydrogenase and related dehydrogenases (Escherichia coli B7A) gi|75231601|ref|ZP_{—}00717959.1|(75231601); and COG1052: Lactate dehydrogenase and related dehydrogenases (Escherichia coli B171) gi|75211308|ref|ZP_{—}00711407.1|(75211308), each sequence associated with the accession number is incorporated herein by reference in its entirety.

Two membrane-bound, FAD-containing enzymes are responsible for the catalysis of fumarate and succinate interconversion; the fumarate reductase is used in anaerobic growth, and the succinate dehydrogenase is used in aerobic growth. Fumarate reductase comprises multiple subunits (e.g., frdA, B, and C in E. coli). Modification of any one of the subunits can result in the desired activity herein. For example, a knockout of frdB, frdC or frdBC is useful in the methods of the disclosure. Frd homologs and variants are known. For example, homologs and variants includes, for example, Fumarate reductase subunit D (Fumarate reductase 13 kDa hydrophobic protein) gi|67463543|sp|P0A8Q3.1|FRDD_{—}ECOLI(67463543); Fumarate reductase subunit C (Fumarate reductase 15 kDa hydrophobic protein) gi|1346037|sp|P20923.2|FRDC_PROVU(1346037); Fumarate reductase subunit D (Fumarate reductase 13 kDa hydrophobic protein) gi|120499|sp|P20924.1|FRDD_PROVU(120499); Fumarate reductase subunit C (Fumarate reductase 15 kDa hydrophobic protein) gi|67463538|sp|P0A8Q0.1|FRDC_{—}ECOLI(67463538); fumarate reductase iron-sulfur subunit (Escherichia coli) gi|145264|gb|AAA23438.1|(145264); fumarate reductase flavoprotein subunit (Escherichia coli) gi|145263|gb|AAA23437.1|(145263); Fumarate reductase flavoprotein subunit gi|37538290|sp|P17412.3|FRDA_WOLSU(37538290); Fumarate reductase flavoprotein subunit gi|120489|sp|P00363.3|FRDA_{—}ECOLI(120489); Fumarate reductase flavoprotein subunit gi|120490|sp|P20922.1|FRDA_PROVU(120490); Fumarate reductase flavoprotein subunit precursor (Flavocytochrome c) (Flavocytochrome c3) (Fcc3) gi|119370087|sp|Q07WU7.2|FRDA_SHEFN(119370087); Fumarate reductase iron-sulfur subunit gi|81175308|sp|P0AC47.2|FRDB_{—}ECOLI(81175308); Fumarate reductase flavoprotein subunit (Flavocytochrome c) (Flavocytochrome c3) (Fcc3) gi|119370088|sp|P0C278.1|FRDA_SHEFR(119370088); Frd operon uncharacterized protein C gi|140663|sp|P20927.1|YFRC_PROVU(140663); Frd operon probable iron-sulfur subunit A gi|140661|sp|P20925.1|YFRA_PROVU(140661); Fumarate reductase iron-sulfur subunit gi|120493|sp|P20921.2|FRDB_PROVU(120493); Fumarate reductase flavoprotein subunit gi|2494617|sp|O06913.2|FRDA_HELPY(2494617); Fumarate reductase flavoprotein subunit precursor (Iron(III)-induced flavocytochrome C3) (Ifc3) gi|13878499|sp|Q9Z4P0.1|FRD2_SHEFN(13878499); Fumarate reductase flavoprotein subunit gi|54041009|sp|P64174.1|FRDA_MYCTU(54041009); Fumarate reductase flavoprotein subunit gi|54037132|sp|P64175.1|FRDA_MYCBO(54037132); Fumarate reductase flavoprotein subunit gi|12230114|sp|Q9ZMP0.1|FRDA_HELPJ(12230114); Fumarate reductase flavoprotein subunit gi|1169737|sp|P44894.1|FRDA_HAEIN(1169737); fumarate reductase flavoprotein subunit (Wolinella succinogenes) gi|13160058|emb|CAA04214.2|(13160058); Fumarate reductase flavoprotein subunit precursor (Flavocytochrome c) (FL cyt) gi|25452947|sp|P83223.2|FRDA_SHEON(25452947); fumarate reductase iron-sulfur subunit (Wolinella succinogenes) gi|2282000|emb|CAA04215.1|(2282000); and fumarate reductase cytochrome b subunit (Wolinella succinogenes) gi|2281998|emb|CAA04213.1|(2281998), each sequence associated with the accession number is incorporated herein by reference in its entirety.

Acetate kinase is encoded in E. coli by ackA. AckA is involved in conversion of acetyl-coA to acetate. Specifically, ackA catalyzes the conversion of acetyl-phophate to acetate. AckA homologs and variants are known. The NCBI database list approximately 1450 polypeptides as bacterial acetate kinases. For example, such homologs and variants include acetate kinase (Streptomyces coelicolor A3(2)) gi|21223784|ref|NP_{—}629563.1|(21223784); acetate kinase (Streptomyces coelicolor A3(2)) gi|6808417|emb|CAB70654.1|(6808417); acetate kinase (Streptococcus pyogenes M1 GAS) gi|15674332|ref|NP_{—}268506.1|(15674332); acetate kinase (Campylobacter jejuni subsp. jejuni NCTC 11168) gi|15792038|ref|NP_{—}281861.1|(15792038); acetate kinase (Streptococcus pyogenes M1 GAS) gi|13621416|gb|AAK33227.1|(13621416); acetate kinase (Rhodopirellula baltica SH 1) gi|32476009|ref|NP_{—}869003.1|(32476009); acetate kinase (Rhodopirellula baltica SH 1) gi|32472045|ref|NP_{—}865039.1|(32472045); acetate kinase (Campylobacter jejuni subsp. jejuni NCTC 11168) gi|112360034|emb|CAL34826.1|(112360034); acetate kinase (Rhodopirellula baltica SH 1) gi|32446553|emb|CAD76388.1|(32446553); acetate kinase (Rhodopirellula baltica SH 1) gi|32397417|emb|CAD72723.1|(32397417); AckA (Clostridium kluyveri DSM 555) gi|153954016|ref|YP_{—}001394781.1|(153954016); acetate kinase (Bifidobacterium longum NCC2705) gi|23465540|ref|NP_{—}696143.1|(23465540); AckA (Clostridium kluyveri DSM 555) gi|146346897|gb|EDK33433.1|(146346897); Acetate kinase (Corynebacterium diphtheriae) gi|38200875|emb|CAE50580.1|(38200875); acetate kinase (Bifidobacterium longum NCC2705) gi|23326203|gb|AAN24779.1|(23326203); Acetate kinase (Acetokinase) gi|67462089|sp|P0A6A3.1|ACKA_{—}ECOLI(67462089); and AckA (Bacillus licheniformis DSM 13) gi|52349315|gb|AAU41949.1|(52349315), the sequences associated with such accession numbers are incorporated herein by reference.

Phosphate acetyltransferase is encoded in E. coli by pta. PTA is involved in conversion of acetate to acetyl-CoA. Specifically, PTA catalyzes the conversion of acetyl-coA to acetyl-phosphate. PTA homologs and variants are known. There are approximately 1075 bacterial phosphate acetyltransferases available on NCBI. For example, such homologs and variants include phosphate acetyltransferase Pta (Rickettsia fells URRWXCa12) gi|67004021|gb|AAY60947.1|(67004021); phosphate acetyltransferase (Buchnera aphidicola str. Cc (Cinara cedri)) gi|116256910|gb|ABJ90592.1|(116256910); pta (Buchnera aphidicola str. Cc (Cinara cedri)) gi|116515056|ref|YP_{—}802685.1|(116515056); pta (Wigglesworthia glossinidia endosymbiont of Glossina brevipalpis) gi|25166135|dbj|BAC24326.1|(25166135); Pta (Pasteurella multocida subsp. multocida str. Pm70) gi|12720993|gb|AAK02789.1|(12720993); Pta (Rhodospirillum rubrum) gi|25989720|gb|AAN75024.1|(25989720); pta (Listeria welshimeri serovar 6b str. SLCC5334) gi|116742418|emb|CAK21542.1|(116742418); Pta (Mycobacterium avium subsp. paratuberculosis K-10) gi|41398816|gb|AAS06435.1|(41398816); phosphate acetyltransferase (pta) (Borrelia burgdorferi B31) gi|15594934|ref|NP_{—}212723.1|(15594934); phosphate acetyltransferase (pta) (Borrelia burgdorferi B31) gi|2688508|gb|AAB91518.1|(2688508); phosphate acetyltransferase (pta) (Haemophilus influenzae Rd KW20) gi|1574131|gb|AAC22857.1|(1574131); Phosphate acetyltransferase Pta (Rickettsia bellii RML369-C) gi|91206026|ref|YP_{—}538381.1|(91206026); Phosphate acetyltransferase Pta (Rickettsia bellii RML369-C) gi|91206025|ref|YP_{—}538380.1|(91206025); phosphate acetyltransferase pta (Mycobacterium tuberculosis F11) gi|148720131|gb|ABR04756.1|(148720131); phosphate acetyltransferase pta (Mycobacterium tuberculosis str. Haarlem) gi|134148886|gb|EBA40931.1|(134148886); phosphate acetyltransferase pta (Mycobacterium tuberculosis C) gi|124599819|gb|EAY58829.1|(124599819); Phosphate acetyltransferase Pta (Rickettsia bellii RML369-C) gi|91069570|gb|ABE05292.1|(91069570); Phosphate acetyltransferase Pta (Rickettsia bellii RML369-C) gi|91069569|gb|ABE05291.1|(91069569); phosphate acetyltransferase (pta) (Treponema pallidum subsp. pallidum str. Nichols) gi|15639088|ref|NP_{—}218534.1|(15639088); and phosphate acetyltransferase (pta) (Treponema pallidum subsp. pallidum str. Nichols) gi|3322356|gb|AAC65090.1|(3322356), each sequence associated with the accession number is incorporated herein by reference in its entirety.

Pyruvate-formate lyase (Formate acetyltransferase) is an enzyme that catalyzes the conversion of pyruvate to acetyl-coA and formate. It is induced by pfl-activating enzyme under anaerobic conditions by generation of an organic free radical and decreases significantly during phosphate limitation. Formate acetyltransferase is encoded in E. coli by pfB. PFLB homologs and variants are known. For examples, such homologs and variants include, for example, Formate acetyltransferase 1 (Pyruvate formate-lyase 1) gi|129879|sp|P09373.2|PFLB_{—}ECOLI(129879); formate acetyltransferase 1 (Yersinia pestis CO92) gi|16121663|ref|NP_{—}404976.1|(16121663); formate acetyltransferase 1 (Yersinia pseudotuberculosis IP 32953) gi|51595748|ref|YP_{—}069939.1|(51595748); formate acetyltransferase 1 (Yersinia pestis biovar Microtus str. 91001) gi|45441037|ref|NP_{—}992576.1|(45441037); formate acetyltransferase 1 (Yersinia pestis CO92) gi|115347142|emb|CAL20035.1|(115347142); formate acetyltransferase 1 (Yersinia pestis biovar Microtus str. 91001) gi|45435896|gb|AAS61453.1|(45435896); formate acetyltransferase 1 (Yersinia pseudotuberculosis IP 32953) gi|51589030|emb|CAH20648.1|(51589030); formate acetyltransferase 1 (Salmonella enterica subsp. enterica serovar Typhi str. CT18) gi|16759843|ref|NP_{—}455460.1|(16759843); formate acetyltransferase 1 (Salmonella enterica subsp. enterica serovar Paratyphi A str. ATCC 9150) gi|56413977|ref|YP_{—}151052.1|(56413977); formate acetyltransferase 1 (Salmonella enterica subsp. enterica serovar Typhi) gi|16502136|emb|CAD05373.1|(16502136); formate acetyltransferase 1 (Salmonella enterica subsp. enterica serovar Paratyphi A str. ATCC 9150) gi|56128234|gb|AAV77740.1|(56128234); formate acetyltransferase 1 (Shigella dysenteriae Sd197) gi|82777577|ref|YP_{—}403926.1|(82777577); formate acetyltransferase 1 (Shigella flexneri 2a str. 2457T) gi|30062438|ref|NP_{—}836609.1|(30062438); formate acetyltransferase 1 (Shigella flexneri 2a str. 2457T) gi|30040684|gb|AAP16415.1|(30040684); formate acetyltransferase 1 (Shigella flexneri 5 str. 8401) gi|110614459|gb|ABF03126.1|(110614459); formate acetyltransferase 1 (Shigella dysenteriae Sd197) gi|81241725|gb|ABB62435.1|(81241725); formate acetyltransferase 1 (Escherichia coli O157:H7 EDL933) gi|12514066|gb|AAG55388.1|AE005279_{—}8(12514066); formate acetyltransferase 1 (Yersinia pestis KIM) gi|22126668|ref|NP_{—}670091.1|(22126668); formate acetyltransferase 1 (Streptococcus agalactiae A909) gi|76787667|ref|YP_{—}330335.1|(76787667); formate acetyltransferase 1 (Yersinia pestis KIM) gi|21959683|gb|AAM86342.1|AE013882_{—}3(21959683); formate acetyltransferase 1 (Streptococcus agalactiae A909) gi|76562724|gb|ABA45308.1|(76562724); formate acetyltransferase 1 (Yersinia enterocolitica subsp. enterocolitica 8081) gi|123441844|ref|YP_{—}001005827.1|(123441844); formate acetyltransferase 1 (Shigella flexneri 5 str. 8401) gi|110804911|ref|YP_{—}688431.1|(110804911); formate acetyltransferase 1 (Escherichia coli UTI89) gi|91210004|ref|YP_{—}539990.1|(91210004); formate acetyltransferase 1 (Shigella boydii Sb227) gi|82544641|ref|YP_{—}408588.1|(82544641); formate acetyltransferase 1 (Shigella sonnei Ss046) gi|74311459|ref|YP_{—}309878.1|(74311459); formate acetyltransferase 1 (Klebsiella pneumoniae subsp. pneumoniae MGH 78578) gi|152969488|ref|YP_{—}001334597.1|(152969488); formate acetyltransferase 1 (Salmonella enterica subsp. enterica serovar Typhi Ty2) gi|29142384|ref|NP_{—}805726.1|(29142384) formate acetyltransferase 1 (Shigella flexneri 2a str. 301) gi|24112311|ref|NP_{—}706821.1|(24112311); formate acetyltransferase 1 (Escherichia coli O157:H7 EDL933) gi|15800764|ref|NP_{—}286778.1|(15800764); formate acetyltransferase 1 (Klebsiella pneumoniae subsp. pneumoniae MGH 78578) gi|150954337|gb|ABR76367.1|(150954337); formate acetyltransferase 1 (Yersinia pestis CA88-4125) gi|149366640|ref|ZP_{—}01888674.1|(149366640); formate acetyltransferase 1 (Yersinia pestis CA88-4125) gi|149291014|gb|EDM41089.1|(149291014); formate acetyltransferase 1 (Yersinia enterocolitica subsp. enterocolitica 8081) gi|122088805|emb|CAL11611.1|(122088805); formate acetyltransferase 1 (Shigella sonnei Ss046) gi|73854936|gb|AAZ87643.1|(73854936); formate acetyltransferase 1 (Escherichia coli UTI89) gi|91071578|gb|ABE06459.1|(91071578); formate acetyltransferase 1 (Salmonella enterica subsp. enterica serovar Typhi Ty2) gi|29138014|gb|AAO69575.1|(29138014); formate acetyltransferase 1 (Shigella boydii Sb227) gi|81246052|gb|ABB66760.1|(81246052); formate acetyltransferase 1 (Shigella flexneri 2a str. 301) gi|24051169|gb|AAN42528.1|(24051169); formate acetyltransferase 1 (Escherichia coli O157:H7 str. Sakai) gi|13360445|dbj|BAB34409.1|(13360445); formate acetyltransferase 1 (Escherichia coli O157:H7 str. Sakai) gi|15830240|ref|NP_{—}309013.1|(15830240); formate acetyltransferase I (pyruvate formate-lyase 1) (Photorhabdus luminescens subsp. laumondii TTO1) gi|36784986|emb|CAE13906.1|(36784986); formate acetyltransferase I (pyruvate formate-lyase 1) (Photorhabdus luminescens subsp. laumondii TTO1) gi|37525558|ref|NP_{—}928902.1|(37525558); formate acetyltransferase (Staphylococcus aureus subsp. aureus Mu50) gi|14245993|dbj|BAB56388.1|(14245993); formate acetyltransferase (Staphylococcus aureus subsp. aureus Mu50) gi|15923216|ref|NP_{—}370750.1|(15923216); Formate acetyltransferase (Pyruvate formate-lyase) gi|81706366|sp|Q7A7X6.1|PFLB_STAAN(81706366); Formate acetyltransferase (Pyruvate formate-lyase) gi|81782287|sp|Q99WZ7.1|PFLB_STAAM(81782287); Formate acetyltransferase (Pyruvate formate-lyase) gi|81704726|sp|Q7A1W9.1|PFLB_STAAW(81704726); formate acetyltransferase (Staphylococcus aureus subsp. aureus Mu3) gi|156720691|dbj|BAF77108.1|(156720691); formate acetyltransferase (Erwinia carotovora subsp. atroseptica SCRI1043) gi|50121521|ref|YP_{—}050688.1|(50121521); formate acetyltransferase (Erwinia carotovora subsp. atroseptica SCRI1043) gi|49612047|emb|CAG75496.1|(49612047); formate acetyltransferase (Staphylococcus aureus subsp. aureus str. Newman) gi|150373174|dbj|BAF66434.1|(150373174); formate acetyltransferase (Shewanella oneidensis MR-1) gi|24374439|ref|NP_{—}718482.1|(24374439); formate acetyltransferase (Shewanella oneidensis MR-1) gi|24349015|gb|AAN55926.1|AE015730_{—}3(24349015); formate acetyltransferase (Actinobacillus pleuropneumoniae serovar 3 str. JL03) gi|165976461|ref|YP_{—}001652054.1|(165976461); formate acetyltransferase (Actinobacillus pleuropneumoniae serovar 3 str. JL03) gi|165876562|gb|ABY69610.1|(165876562); formate acetyltransferase (Staphylococcus aureus subsp. aureus MW2) gi|21203365|dbj|BAB94066.1|(21203365); formate acetyltransferase (Staphylococcus aureus subsp. aureus N315) gi|13700141|dbj|BAB41440.1|(13700141); formate acetyltransferase (Staphylococcus aureus subsp. aureus str. Newman) gi|151220374|ref|YP_{—}001331197.1|(151220374); formate acetyltransferase (Staphylococcus aureus subsp. aureus Mu3) gi|156978556|ref|YP_{—}001440815.1|(156978556); formate acetyltransferase (Synechococcus sp. JA-2-3B′a(2-13)) gi|86607744|ref|YP_{—}476506.1|(86607744); formate acetyltransferase (Synechococcus sp. JA-3-3Ab) gi|86605195|ref|YP_{—}473958.1|(86605195); formate acetyltransferase (Streptococcus pneumoniae D39) gi|116517188|ref|YP_{—}815928.1|(116517188); formate acetyltransferase (Synechococcus sp. JA-2-3B′a(2-13)) gi|86556286|gb|ABD01243.1|(86556286); formate acetyltransferase (Synechococcus sp. JA-3-3Ab) gi|86553737|gb|ABC98695.1|(86553737); formate acetyltransferase (Clostridium novyi NT) gi|118134908|gb|ABK61952.1|(118134908); formate acetyltransferase (Staphylococcus aureus subsp. aureus MRSA252) gi|49482458|ref|YP_{—}039682.1|(49482458); and formate acetyltransferase (Staphylococcus aureus subsp. aureus MRSA252) gi|49240587|emb|CAG39244.1|(49240587), each sequence associated with the accession number is incorporated herein by reference in its entirety.

Alpha isopropylmalate synthase (EC 2.3.3.13, sometimes referred to a 2-isopropylmalate synthase, alpha-IPM synthetase) catalyzes the condensation of the acetyl group of acetyl-CoA with 3-methyl-2-oxobutanoate (2-oxoisovalerate) to form 3-carboxy-3-hydroxy-4-methylpentanoate (2-isopropylmalate). Alpha isopropylmalate synthase is encoded in E. coli by leuA. LeuA homologs and variants are known. For example, such homologs and variants include, for example, 2-isopropylmalate synthase (Corynebacterium glutamicum) gi|452382|emb|CAA50295.1|(452382); 2-isopropylmalate synthase (Escherichia coli K12) gi|16128068|ref|NP_{—}414616.1|(16128068); 2-isopropylmalate synthase (Escherichia coli K12) gi|1786261|gb|AAC73185.1|(1786261); 2-isopropylmalate synthase (Arabidopsis thaliana) gi|15237194|ref|NP_{—}197692.1|(15237194); 2-isopropylmalate synthase (Arabidopsis thaliana) gi|42562149|ref|NP_{—}173285.2|(42562149); 2-isopropylmalate synthase (Arabidopsis thaliana) gi|15221125|ref|NP_{—}177544.1|(15221125); 2-isopropylmalate synthase (Streptomyces coelicolor A3(2)) gi|32141173|ref|NP_{—}733575.1|(32141173); 2-isopropylmalate synthase (Rhodopirellula baltica SH 1) gi|32477692|ref|NP_{—}870686.1|(32477692); 2-isopropylmalate synthase (Rhodopirellula baltica SH 1) gi|32448246|emb|CAD77763.1|(32448246); 2-isopropylmalate synthase (Akkermansia muciniphila ATCC BAA-835) gi|166241432|gb|EDR53404.1|(166241432); 2-isopropylmalate synthase (Herpetosiphon aurantiacus ATCC 23779) gi|159900959|ref|YP_{—}001547206.1|(159900959); 2-isopropylmalate synthase (Dinoroseobacter shibae DFL 12) gi|159043149|ref|YP_{—}001531943.1|(159043149); 2-isopropylmalate synthase (Salinispora arenicola CNS-205) gi|159035933|ref|YP_{—}001535186.1|(159035933); 2-isopropylmalate synthase (Clavibacter michiganensis subsp. michiganensis NCPPB 382) gi|148272757|ref|YP_{—}001222318.1|(148272757); 2-isopropylmalate synthase (Escherichia coli B) gi|124530643|ref|ZP_{—}01701227.1|(124530643); 2-isopropylmalate synthase (Escherichia coli C str. ATCC 8739) gi|124499067|gb|EAY46563.1|(124499067); 2-isopropylmalate synthase (Bordetella pertussis Tohama I) gi|33591386|refNP_{—}879030.1|(33591386); 2-isopropylmalate synthase (Polynucleobacter necessarius STIR1) gi|164564063|ref|ZP_{—}02209880.1|(164564063); 2-isopropylmalate synthase (Polynucleobacter necessarius STIR1) gi|164506789|gb|EDQ94990.1|(164506789); and 2-isopropylmalate synthase (Bacillus weihenstephanensis KBAB4) gi|163939313|ref|YP_{—}001644197.1|(163939313), any sequence associated with the accession number is incorporated herein by reference in its entirety.

BCAA aminotransferases catalyze the formation of branched chain amino acids (BCAA). A number of such aminotransferases are known and are exemplified by ilvE in E. coli. Exemplary homologs and variants include sequences designated by the following accession numbers: ilvE (Microcystis aeruginosa PCC 7806) gi|159026756|emb|CAO86637.1|(159026756); IlvE (Escherichia coli) gi|87117962|gb|ABD20288.1|(87117962); IlvE (Escherichia coli) gi|87117960|gb|ABD20287.1|(87117960); IlvE (Escherichia coli) gi|87117958|gb|ABD20286.1|(87117958); IlvE (Shigella flexneri) gi|87117956|gb|ABD20285.1|(87117956); IlvE (Shigella flexneri) gi|87117954|gb|ABD20284.1|(87117954); IlvE (Shigella flexneri) gi|87117952|gb|ABD20283.1|(87117952); IlvE (Shigella flexneri) gi|87117950|gb|ABD20282.1|(87117950); IlvE (Shigella flexneri) gi|87117948|gb|ABD20281.1|(87117948); IlvE (Shigella flexneri) gi|87117946|gb|ABD20280.1|(87117946); IlvE (Shigella flexneri) gi|87117944|gb|ABD20279.1|(87117944); IlvE (Shigella flexneri) gi|87117942|gb|ABD20278.1|(87117942); IlvE (Shigella flexneri) gi|87117940|gb|ABD20277.1|(87117940); IlvE (Shigella flexneri) gi|87117938|gb|ABD20276.1|(87117938); IlvE (Shigella dysenteriae) gi|87117936|gb|ABD20275.1|(87117936); IlvE (Shigella dysenteriae) gi|87117934|gb|ABD20274.1|(87117934); IlvE (Shigella dysenteriae) gi|87117932|gb|ABD20273.1|(87117932); IlvE (Shigella dysenteriae) gi|87117930|gb|ABD20272.1|(87117930); and IlvE (Shigella dysenteriae) gi|87117928|gb|ABD20271.1|(87117928), each sequence associated with the accession number is incorporated herein by reference.

Tyrosine aminotransferases catalyzes transamination for both dicarboxylic and aromatic amino-acid substrates. A tyrosine aminotransferase of E. coli is encoded by the gene tyrB. TyrB homologs and variants are known. For example, such homologs and variants include tyrB (Bordetella petrii) gi|163857093|ref|YP_{—}001631391.1|(163857093); tyrB (Bordetella petrii) gi|163260821|emb|CAP43123.1|(163260821); aminotransferase gi|551844|gb|AAA24704.1|(551844); aminotransferase (Bradyrhizobium sp. BTAi1) gi|146404387|gb|ABQ32893.1|(146404387); tyrosine aminotransferase TyrB (Salmonella enterica) gi|4775574|emb|CAB40973.2|(4775574); tyrosine aminotransferase (Salmonella typhimurium LT2) gi|16422806|gb|AAL23072.1|(16422806); and tyrosine aminotransferase gi|148085|gb|AAA24703.1|(148085), each sequence of which is incorporated herein by reference.

Pyruvate oxidase catalyzes the conversion of pyruvate to acetate and CO₂. In E. coli, pyruvate oxidase is encoded by poxB. PoxB and homologs and variants thereof include, for example, pyruvate oxidase; PoxB (Escherichia coli) gi|685128|gb|AAB31180.1∥bbm|348451|bbs|154716(685128); PoxB (Pseudomonas fluorescens) gi|32815820|gb|AAP88293.1|(32815820); poxB (Escherichia coli) gi|25269169|emb|CAD57486.1|(25269169); pyruvate dehydrogenase (Salmonella enterica subsp. enterica serovar Typhi) gi|16502101|emb|CAD05337.1|(16502101); pyruvate oxidase (Lactobacillus plantarum) gi|41691702|gb|AAS10156.1|(41691702); pyruvate dehydrogenase (Bradyrhizobium japonicum) gi|20257167|gb|AAM12352.1|(20257167); pyruvate dehydrogenase (Yersinia pestis KIM) gi|22126698|ref|NP_{—}670121.1|(22126698); pyruvate dehydrogenase (cytochrome) (Yersinia pestis biovar Antigua str. B42003004) gi|166211240|ref|ZP_{—}02237275.1|(166211240); pyruvate dehydrogenase (cytochrome) (Yersinia pestis biovar Antigua str. B42003004) gi|166207011|gb|EDR51491.1|(166207011); pyruvate dehydrogenase (Pseudomonas syringae pv. tomato str. DC3000) gi|28869703|ref|NP_{—}792322.1|(28869703); pyruvate dehydrogenase (Salmonella typhimurium LT2) gi|16764297|ref|NP_{—}459912.1|(16764297); pyruvate dehydrogenase (Salmonella enterica subsp. enterica serovar Typhi str. CT18) gi|16759808|ref|NP_{—}455425.1|(16759808); pyruvate dehydrogenase (cytochrome) (Coxiella burnetii Dugway 5J108-111) gi|154706110|ref|YP_{—}001424132.1|(154706110); pyruvate dehydrogenase (Clavibacter michiganensis subsp. michiganensis NCPPB 382) gi|148273312|ref|YP_{—}001222873.1|(148273312); pyruvate oxidase (Lactobacillus acidophilus NCFM) gi|58338213|ref|YP_{—}194798.1|(58338213); and pyruvate dehydrogenase (Yersinia pestis CO92) gi|16121638|ref|NP_{—}404951.1|(16121638), the sequences of each accession number are incorporated herein by reference.

L-threonine 3-dehydrogenase (EC 1.1.1.103) catalyzes the conversion of L-threonine to L-2-amino-3-oxobutanoate. The gene tdh encodes an L-threonine 3-dehydrogenase. There are approximately 700 L-threonine 3-dehydrogenases from bacterial organism recognized in NCBI. Various homologs and variants of tdh include, for example, L-threonine 3-dehydrogenase gi|135560|sp|P07913.1|TDH_{—}ECOLI(135560); L-threonine 3-dehydrogenase gi|166227854|sp|A4TSC6.1|TDH_YERPP(166227854); L-threonine 3-dehydrogenase gi|166227853|sp|A1JHX8.1|TDH_YERE8(166227853); L-threonine 3-dehydrogenase gi|166227852|sp|A6UBM6.1|TDH_SINMW(166227852); L-threonine 3-dehydrogenase gi|166227851|sp|A1RE07.1|TDH_SHESW(166227851); L-threonine 3-dehydrogenase gi|166227850|sp|A0L2Q3.1|TDH_SHESA(166227850); L-threonine 3-dehydrogenase gi|166227849|sp|A4YCC5.1|TDH_SHEPC(166227849); L-threonine 3-dehydrogenase gi|166227848|sp|A3QJC8.1|TDH_SHELP(166227848); L-threonine 3-dehydrogenase gi|166227847|sp|A6WUG6.1|TDH_SHEB8(166227847); L-threonine 3-dehydrogenase gi|166227846|sp|A3CYN0.1|TDH_SHEB5(166227846); L-threonine 3-dehydrogenase gi|166227845|sp|A1S1Q3.1|TDH_SHEAM(166227845); L-threonine 3-dehydrogenase gi|166227844|sp|A4FND4.1|TDH_SACEN(166227844); L-threonine 3-dehydrogenase gi|166227843|sp|A1SVW5.1|TDH_PSYIN(166227843); L-threonine 3-dehydrogenase gi|166227842|sp|A51GK7.1|TDH_LEGPC(166227842); L-threonine 3-dehydrogenase gi|166227841|sp|A6TFL2.1|TDH_KLEP7(166227841); L-threonine 3-dehydrogenase gi|166227840|sp|A4IZ92.1|TDH_FRATW(166227840); L-threonine 3-dehydrogenase gi|166227839|sp|A0Q5K3.1|TDH_FRATN(166227839); L-threonine 3-dehydrogenase gi|166227838|sp|A7NDM9.1|TDH_FRATF(166227838); L-threonine 3-dehydrogenase gi|166227837|sp|A7MID0.1|TDH_ENTS8(166227837); and L-threonine 3-dehydrogenase gi|166227836|sp|A1AHF3.1|TDH_ECOK1(166227836), the sequences associated with each accession number are incorporated herein by reference.

Acetohydroxy acid synthases (e.g. ilvH) and acetolactate synthases (e.g., alsS, ilvB, ilvI) catalyze the synthesis of the branched-chain amino acids (valine, leucine, and isoleucine). IlvH encodes an acetohydroxy acid synthase in E. coli (see, e.g., acetohydroxy acid synthase AHAS III (IlvH) (Escherichia coli) gi|40846|emb|CAA38855.1|(40846), incorporated herein by reference). Homologs and variants as well as operons comprising ilvH are known and include, for example, ilvH (Microcystis aeruginosa PCC 7806)gi|159026908|emb|CAO89159.1|(159026908); IlvH (Bacillus amyloliquefaciens FZB42) gi|154686966|ref|YP_{—}001422127.1|(154686966); IlvH (Bacillus amyloliquefaciens FZB42) gi|154352817|gb|ABS74896.1|(154352817); IlvH (Xenorhabdus nematophila) gi|131054140|gb|ABO32787.1|(131054140); IlvH (Salmonella typhimurium) gi|7631124|gb|AAF65177.1|AF117227_{—}2(7631124), ilvN (Listeria innocua) gi|16414606|emb|CAC97322.1|(16414606); ilvN (Listeria monocytogenes) gi|16411438|emb|CAD00063.1|(16411438); acetohydroxy acid synthase (Caulobacter crescentus) gi|408939|gb|AAA23048.1|(408939); acetohydroxy acid synthase I, small subunit (Salmonella enterica subsp. enterica serovar Typhi) gi|16504830|emb|CAD03199.1|(16504830); acetohydroxy acid synthase, small subunit (Tropheryma whipplei TW0827) gi|28572714|ref|NP_{—}789494.1|(28572714); acetohydroxy acid synthase, small subunit (Tropheryma whipplei TW0827) gi|28410846|emb|CAD67232.1|(28410846); acetohydroxy acid synthase I, small subunit (Salmonella enterica subsp. enterica serovar Paratyphi A str. ATCC 9150) gi|56129933|gb|AAV79439.1|(56129933); acetohydroxy acid synthase small subunit; acetohydroxy acid synthase, small subunit gi|551779|gb|AAA62430.1|(551779); acetohydroxy acid synthase I, small subunit (Salmonella enterica subsp. enterica serovar Typhi Ty2) gi|29139650|gb|AAO71216.1|(29139650); acetohydroxy acid synthase small subunit (Streptomyces cinnamonensis) gi|5733116|gb|AAD49432.1|AF175526_{—}1(5733116); acetohydroxy acid synthase large subunit; and acetohydroxy acid synthase, large subunit gi|400334|gb|AAA62429.1|(400334), the sequences associated with the accession numbers are incorporated herein by reference. Acetolactate synthase genes include alsS and ilvI. Homologs of ilvI and alsS are known and include, for example, acetolactate synthase small subunit (Bifidobacterium longum NCC2705) gi|23325489|gb|AAN24137.1|(23325489); acetolactate synthase small subunit (Geobacillus stearothermophilus) gi|19918933|gb|AAL99357.1|(19918933); acetolactate synthase (Azoarcus sp. BH72) gi|119671178|emb|CAL95091.1|(119671178); Acetolactate synthase small subunit (Corynebacterium diphtheriae) gi|38199954|emb|CAE49622.1|(38199954); acetolactate synthase (Azoarcus sp. BH72) gi|119669739|emb|CAL93652.1|(119669739); acetolactate synthase small subunit (Corynebacterium jeikeium K411) gi|68263981|emb|CAI37469.1|(68263981); acetolactate synthase small subunit (Bacillus subtilis) gi|1770067|emb|CAA99562.1|(1770067); Acetolactate synthase isozyme 1 small subunit (AHAS-I) (Acetohydroxy-acid synthase I small subunit) (ALS-I) gi|83309006|sp|P0ADF8.1|ILVN_{—}ECOLI(83309006); acetolactate synthase large subunit (Geobacillus stearothermophilus) gi|19918932|gb|AAL99356.1|(19918932); and Acetolactate synthase, small subunit (Thermoanaerobacter tengcongensis MB4) gi|20806556|ref|NP_{—}621727.1|(20806556), the sequences associated with the accession numbers are incorporated herein by reference. There are approximately 1120 ilvB homologs and variants listed in NCBI.

Acetohydroxy acid isomeroreductase is the second enzyme in parallel pathways for the biosynthesis of isoleucine and valine. IlvC encodes an acetohydroxy acid isomeroreductase in E. coli. Homologs and variants of ilvC are known and include, for example, acetohydroxyacid reductoisomerase (Schizosaccharomyces pombe 972h-) gi|162312317|ref|NP_{—}001018845.2|(162312317); acetohydroxyacid reductoisomerase (Schizosaccharomyces pombe) gi|3116142|emb|CAA18891.1|(3116142); acetohydroxyacid reductoisomerase (Saccharomyces cerevisiae YJM789) gi|151940879|gb|EDN59261.1|(151940879); Ilv5p: acetohydroxyacid reductoisomerase (Saccharomyces cerevisiae) gi|609403|gb|AAB67753.1|(609403); ACL198Wp (Ashbya gossypii ATCC 10895) gi|45185490|ref|NP_{—}983206.1|(45185490); ACL198Wp (Ashbya gossypii ATCC 10895) gi|44981208|gb|AAS51030.1|(44981208); acetohydroxy-acid isomeroreductase; Ilv5x (Saccharomyces cerevisiae) gi|957238|gb|AAB33579.1∥bbm|369068|bbs|165406(957238); acetohydroxy-acid isomeroreductase; Ilv5g (Saccharomyces cerevisiae) gi|957236|gb|AAB33578.1∥bbm|369064|bbs|165405(957236); and ketol-acid reductoisomerase (Schizosaccharomyces pombe) gi|2696654|dbj|BAA24000.1|(2696654), each sequence associated with the accession number is incorporated herein by reference.

Dihydroxy-acid dehydratases catalyzes the fourth step in the biosynthesis of isoleucine and valine, the dehydratation of 2,3-dihydroxy-isovaleic acid into alpha-ketoisovaleric acid. IlvD and ilv3 encode a dihydroxy-acid dehydratase. Homologs and variants of dihydroxy-acid dehydratases are known and include, for example, IlvD (Mycobacterium leprae) gi|2104594|emb|CAB08798.1|(2104594); dihydroxy-acid dehydratase (Tropheryma whipplei TW0827) gi|28410848|emb|CAD67234.1|(28410848); dihydroxy-acid dehydratase (Mycobacterium leprae) gi|13093837|emb|CAC32140.1|(13093837); dihydroxy-acid dehydratase (Rhodopirellula baltica SH 1) gi|32447871|emb|CAD77389.1|(32447871); and putative dihydroxy-acid dehydratase (Staphylococcus aureus subsp. aureus MRSA252) gi|49242408|emb|CAG41121.1|(49242408), each sequence associated with the accession numbers are incorporated herein by reference.

2-ketoacid decarboxylases catalyze the conversion of a 2-ketoacid to the respective aldehyde. For example, 2-ketoisovalerate decarboxylase catalyzes the conversion of 2-ketoisovalerate to isobutyraldehyde. A number of 2-ketoacid decarboxylases are known and are exemplified by the pdc, pdc1, pdc5, pdc6, aro10, thI3, kdcA and kivd genes. Exemplary homologs and variants useful for the conversion of a 2-ketoacid to the respective aldehyde comprise sequences designated by the following accession numbers and identified enzymatic activity: gi|44921617|gb|AAS49166.1|branched-chain alpha-keto acid decarboxylase (Lactococcus lactis); gi|15004729|ref|NP_{—}149189.1|Pyruvate decarboxylase (Clostridium acetobutylicum ATCC 824); gi|82749898|ref|YP_{—}415639.1|probable pyruvate decarboxylase (Staphylococcus aureus RF122); gi|77961217|ref|ZP_{—}00825060.1|COG3961: Pyruvate decarboxylase and related thiamine pyrophosphate-requiring enzymes (Yersinia mollaretii ATCC 43969); gi|71065418|ref|YP_{—}264145.1|putative pyruvate decarboxylase (Psychrobacter arcticus 273-4); gi|16761331|ref|NP_{—}456948.1|putative decarboxylase (Salmonella enterica subsp. enterica serovar Typhi str. CT18); gi|93005792|ref|YP_{—}580229.1|Pyruvate decarboxylase (Psychrobacter cryohalolentis K5); gi|23129016|ref|ZP_{—}00110850.1|COG3961: Pyruvate decarboxylase and related thiamine pyrophosphate-requiring enzymes (Nostoc punctiforme PCC 73102); gi|16417060|gb|AAL18557.1|AF354297_{—}1 pyruvate decarboxylase (Sarcina ventriculi); gi|15607993|ref|NP_{—}215368.1|PROBABLE PYRUVATE OR INDOLE-3-PYRUVATE DECARBOXYLASE PDC (Mycobacterium tuberculosis H37Rv); gi|41406881|ref|NP_{—}959717.1|Pdc (Mycobacterium avium subsp. paratuberculosis K-10); gi|91779968|ref|YP_{—}555176.1|putative pyruvate decarboxylase (Burkholderia xenovorans LB400); gi|15828161|ref|NP_{—}302424.1|pyruvate (or indolepyruvate) decarboxylase (Mycobacterium leprae TN); gi|118616174|ref|YP_{—}904506.1|pyruvate or indole-3-pyruvate decarboxylase Pdc (Mycobacterium ulcerans Agy99); gi|67989660|ref|NP_{—}001018185.1|hypothetical protein SPAC3H8.01 (Schizosaccharomyces pombe 972h-); gi|21666011|gb|AAM73540.1|AF282847_{—}1 pyruvate decarboxylase PdcB (Rhizopus oryzae); gi|69291130|ref|ZP_{—}00619161.1|Pyruvate decarboxylase:Pyruvate decarboxylase (Kineococcus radiotolerans SRS30216); gi|66363022|ref|XP_{—}628477.1|pyruvate decarboxylase (Cryptosporidium parvum Iowa II); gi|70981398|ref|XP_{—}731481.1|pyruvate decarboxylase (Aspergillus fumigatus Af293); gi|121704274|ref|XP_{—}001270401.1|pyruvate decarboxylase, putative (Aspergillus clavatus NRRL 1); gi|119467089|ref|XP_{—}001257351.1|pyruvate decarboxylase, putative (Neosartorya fischeri NRRL 181); gi|26554143|ref|NP_{—}758077.1|pyruvate decarboxylase (Mycoplasma penetrans HF-2); gi|21666009|gb|AAM73539.1|AF282846_{—}1 pyruvate decarboxylase PdcA (Rhizopus oryzae).

Alcohol dehydrogenases (adh) catalyze the final step of amino acid catabolism, conversion of an aldehyde to a long chain or complex alcohol. Various adh genes are known in the art. As indicated herein adh1 homologs and variants include, for example, adh2, adh3, adh4, adh5, adh 6 and sfa1 (see, e.g., SFA (Saccharomyces cerevisiae) gi|288591|emb|CAA48161.1|(288591); the sequence associated with the accession number is incorporated herein by reference).

Citramalate synthase catalyzes the condensation of pyruvate and acetate. CimA encodes a citramalate synthase. Homologs and variants are known and include, for example, citramalate synthase (Leptospira biflexa serovar Patoc) gi|116664687|gb|ABK13757.1|(116664687); citramalate synthase (Leptospira biflexa serovar Monteralerio) gi|116664685|gb|ABK13756.1|(116664685); citramalate synthase (Leptospira interrogans serovar Hebdomadis) gi|116664683|gb|ABK13755.1|(116664683); citramalate synthase (Leptospira interrogans serovar Pomona) gi|116664681|gb|ABK13754.1|(116664681); citramalate synthase (Leptospira interrogans serovar Australis) gi|116664679|gb|ABK13753.1|(116664679); citramalate synthase (Leptospira interrogans serovar Autumnalis) gi|116664677|gb|ABK13752.1|(116664677); citramalate synthase (Leptospira interrogans serovar Pyrogenes) gi|116664675|gb|ABK13751.1|(116664675); citramalate synthase (Leptospira interrogans serovar Canicola) gi|116664673|gb|ABK13750.1|(116664673); citramalate synthase (Leptospira interrogans serovar Lai) gi|116664671|gb|ABK13749.1|(116664671); CimA (Leptospira meyeri serovar Semaranga) gi|119720987|gb|ABL98031.1|(119720987); (R)-citramalate synthase gi|2492795|sp|Q58787.1|CIMA_METJA(2492795); (R)-citramalate synthase gi|22095547|sp|P58966.1|CIMA_METMA(22095547); (R)-citramalate synthase gi|22001554|sp|Q8TJJ1.1|CIMA_METAC(22001554); (R)-citramalate synthase gi|22001553|sp|O26819.1|CIMA_METTH(22001553); (R)-citramalate synthase gi|22001555|sp|Q8TYB1.1|CIMA_METKA(22001555); (R)-citramalate synthase (Methanococcus maripaludis S2) gi|45358581|ref|NP_{—}988138.1|(45358581); (R)-citramalate synthase (Methanococcus maripaludis S2) gi|44921339|emb|CAF30574.1|(44921339); and similar to (R)-citramalate synthase (Candidatus Kuenenia stuttgartiensis) gi|91203541|emb|CAJ71194.1|(91203541), each sequence associated with the foregoing accession numbers is incorporated herein by reference.

Several thousand Ribulose-1,5-bisphosphate carbxylaseoxygenase and other CO₂ fixation enzymes are known and their sequences are readily available in the art using various search criteria and web-sites. For example, the methods and compositions of the disclosure may utilize Ribulose-1,5-bisphosphate carboxylaseoxygenase (RubisCo)—small subunit—cbbS, Ribulose-1,5-bisphosphate carbyxlaseoxygenase (RubisCo)—large subunit cbbL, Rubisco activase, rbcL, rbcS and variants and homologs thereof in the disclosure. In yet other related embodiments, the engineered can further comprise engineered rbcL nucleic acid, engineered rbcS nucleic acid, and engineered phosphoribulokinase. Rubisco polypeptides of the useful in the disclosure include Rubisco large subunit polypeptides (“rbcL”), Rubisco small subunit polypeptides (“rbcS”), and Rubisco large/small polypeptides (“rbcLS”). Large and small subunits may be combined in different combinations with each other together in a single enzyme having Rubisco specific activity. Alternatively, the large and small subunits of the may be combined with the large and small subunits from a wild type Rubisco polypeptides to form a polypeptide having Rubisco activity. Exemplary ribulose-1,5-bisphosophate carboxylase/oxygenases include spinach form I Rubisco Spinacia oleracea; gi:7636117; CAB88737, Archaeoglobus fulgidus DSM 4304 rbcL-1 (gi:2648975; AAB86661); Sinorhizobium meliloti 1021 (gi:15140252; CAC48779); Mesorhizobium loti MAFF303099 (gi:14026595; BAB53192); Chlorobium limicola f. thiosulfatophilum (gi:13173182; AAK14332); C. tepidum TLS (gi:21647784; AAM72993); R. palustris (gi:78490428; ZP_{—}00842677); R. palustris (gi:77687805; ZP_{—}00802991); R. rubrum (gi:48764419; ZP_{—}00268971); Bordetella bronchiseptica RB50 (gi:33567621; CAE31534); Burkholderia fungorum LB400 (gi:48788861; ZP_{—}00284840); B. clausii KSM-K16 (gi:56909783; BAD64310); Bacillus thuringiensis serovar konkukian strain 97-27 (gi:49333072; AAT63718); Geobacillus kaustophilus HTA426 (gi:56379330; BAD75238); Bacillus licheniformis ATCC14580 (gi:52003120; AAU23062); Bacillus anthracis strain A2012 (gi:65321428; ZP_{—}00394387); Bacillus cereus E33L (gi:51974924; AAU16474); B. subtilis subsp. subtilis strain 168 (gi:2633730; CAB13232). Accession numbers are from GenBank and sequences associated with those accession numbers are incorporated herein by reference. In addition, variants comprising RuBisCo activity and having at least 85%, 90%, 95%, 98%, 99% identity to any of the foregoing sequences is also encompassed by the disclosure.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Q-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

### EXAMPLES

### Cloning Procedure

The genes kivd (Lactococcus lactis), adhA (Lactococcus lactis), adh2 (Saccharomyces cerevisiae), and yqhD (Escherichia. coli) were amplified using genomic DNA of appropriate organisms. The kivd-adhA, kivd-adh2, and kivd-yqhD artificial operons were then made by SOE (splicing by overlap extension) PCR with ribosome binding sites in front of each gene. The operons were inserted and digested with BspEI and NcoI and inserted into the broadhost-range vector pBHR 1(MoBiTec, Gottingen, Germany).

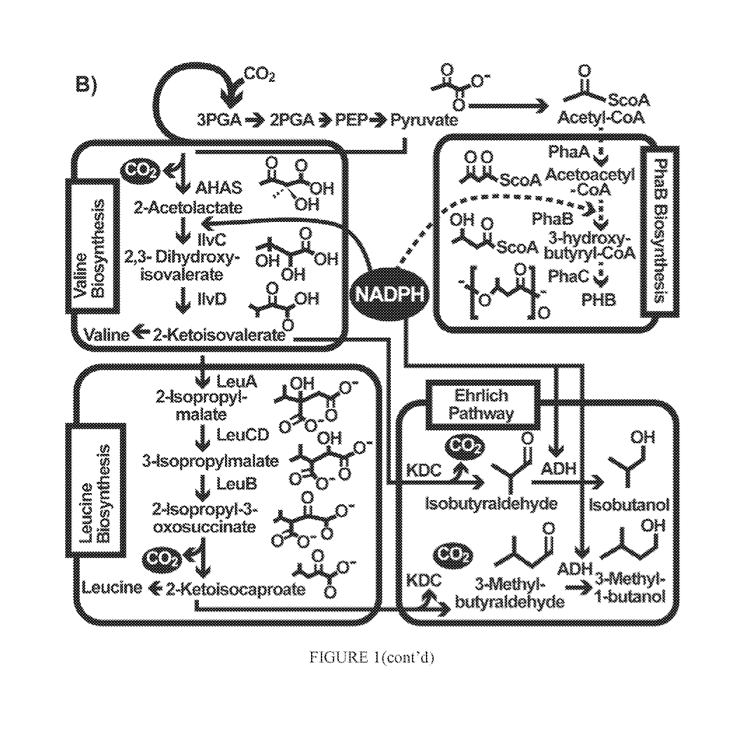

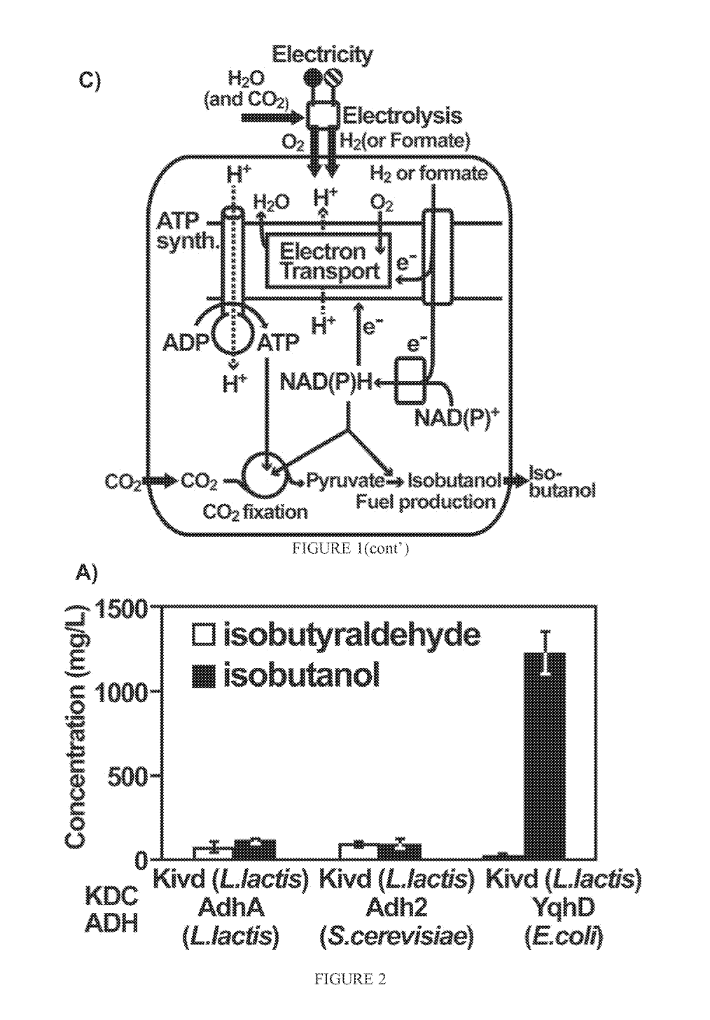

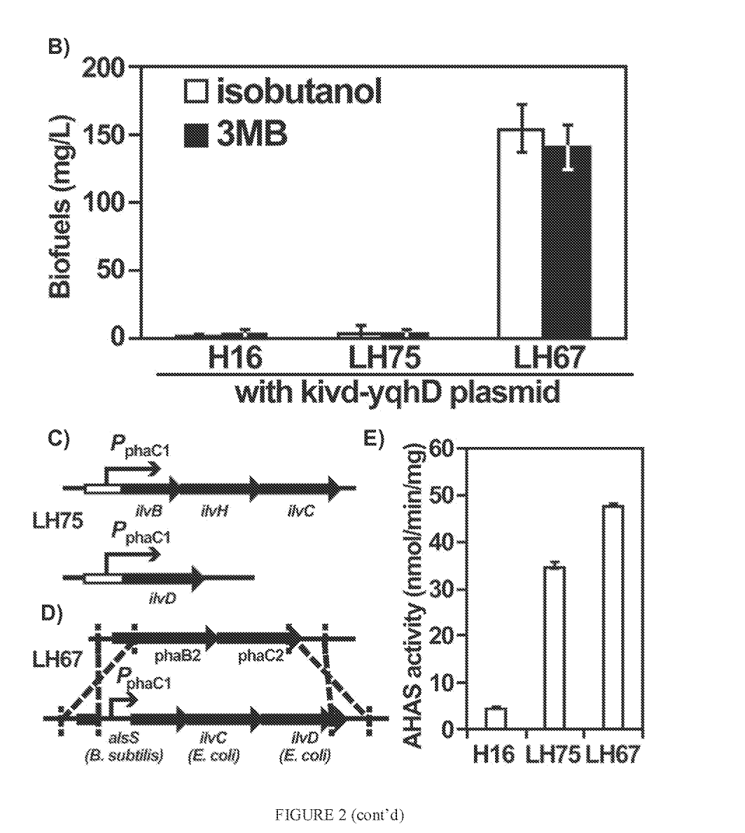

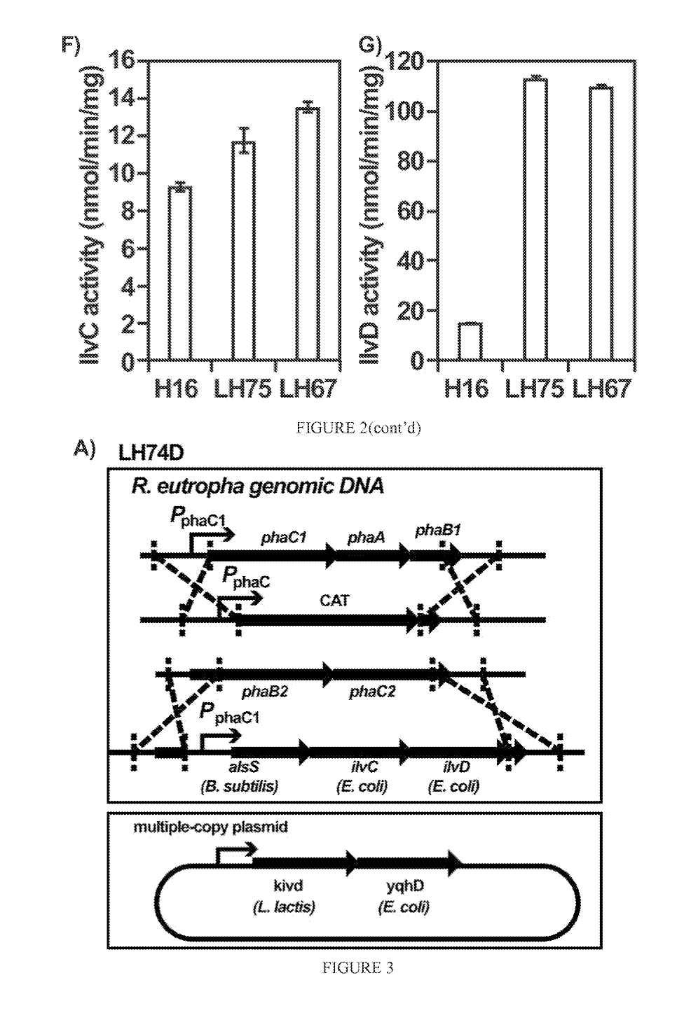

The 500 bp DNA fragments upstream of Ralstonia eutropha phaB2 gene and downstream of phaC2 gene were amplified from genomic DNA and assembled with SOE with a linker region containing NotI and NcoI enzyme sites in between. The assembly product was digested with MluI and XbaI and inserted into the conjugation vector pNHG 1 (34) to form pLH50. The artificial operon containing alsS (Bacillus subtilis), ilvC (E. coli), and ilvD (E. coli) was amplified from plasmid pSA69 (Atsumi et al., Nature 451, 86 (2008)) and assembled with the 836 bp phaC1 promoter region amplified from R. eutropha genomic DNA by SOE. This fragment was then inserted into the SacI site of pLH50 to form plasmid pLH63 (Table 6). The pLH63 was used to perform conjugation by the reported method (11). After double-crossover selection on sucrose, the strain with alsS, ilvC, and ilvD overexpression was confirmed by PCR of genomic DNA and enzyme assays using cell lysate (FIG. 2A-B).

The 1000 bp DNA fragments upstream of R. eutropha ilvB gene and from 1-1000 bp of ilvB gene open reading frame were amplified from genomic DNA and assembled with the phaC1 promoter region by SOE. The assembly product was inserted into NdeI and XbaI sites of pNHG 1. The phaC1 promoter knock-in plasmid for ilvD gene was constructed similarly.

The 1000 bp DNA fragments upstream of R. eutropha phaC1 gene and downstream of phaB1 gene were amplified from genomic DNA and assembled with the chloramphenicol acetyltransferase (CAT) gene by SOE. The assembly product was inserted into MluI and XbaI sites of pNHG 1. The DNA fragments from −500 bp to 150 bp relative to the katG, sodC, and NorA gene open reading frame of R. eutropha were amplified from the genomic DNA and assembled with the lacZ (β-galactosidase) gene using SOE. The resulting products were then inserted into the BspEI and NcoI sites of broad-host-range vector pBHR 1. The transcription direction of lacZ genes was the opposite of the CAT promoter in the plasmid.

The PHB biosynthesis genes were knocked out by chromosomal replacement with a chloramphenicol acetyltransferase (CAT) cassette. The −448 bp to +146 bp DNA fragment relative to R. eutropha phaC1 gene start codon and 500 bp downstream of phaB1 gene were amplified from genomic DNA. The PCR products were assembled by SOE with the chloramphenicol acetyltransferase (CAT) gene with an added ribosome binding site. The assembly product was inserted into MluI and XbaI sites of pNHG1, resulting plasmid pLH51 (Table 6). The plasmid was then introduced into the above-mentioned alsS, ilvC and ilvD overexpression strain by conjugation. After double-crossover selection, the resulting strain was confirmed by PCR and named LH67 (Table 6).

TABLE 6
Plasmids and Strains used:

| | | Reference |
|---|---|---|
| | Description | or Source |
| Plasmid | | |
| pSA69 | P_{L}LacO1: alS-ilvC-ilvD | Atsumi |
| pBHR1 | broad-host-range vector | MoBiTec, |
| | | Göttingen, |
| | | Germany |
| pNHG1 | suicide vector containing sucB | Jeffke et al |
| pLH50 | pNHG1 with homologous regions for making | this study |
| | knockout ΔphaB2C2 | |
| pLH51 | pNHG1 with ΔphaC1AB1::CAT | this study |
| pLH63 | pNHG1 with ΔphaB2C::PphaC1:alsS-ilvC-ilvD | this study |
| pYL22 | pBHR1 with ΔCAT::klvd-yqhD | |
| pLH129 | pBHR1 with P_{katG}:lacZ | this study |
| pLH130 | pBHR1 with P_{nor A}:lacZ | this study |
| pLH131 | pBHR1 with P_{sodC}:lacZ | this study |
| Strain | | |
| XL-1 Blue | Escherichia coli strain used in cloning and | Stratagene. |
| | growth study | La Jolla, |
| | | CA |
| S17-1 | E. coli strain used in conjugation | ATCC |
| H16 | Ralstonia eutopha wild type | A gift from |
| | | Dr. Botho |
| | | Bowien |
| LH67 | H16 with ΔphaB2C2::P_{phaCl}:alsS-ilvC-ilvD. | this study |
| | ΔphaC1AB1::CAT | |
| LH74D | LH67 transformed with pYL22 | this study |
| LH118 | H16 transformed with pLH129 | this study |
| LH119 | H16 transformed with pLH130 | this study |
| LH120 | H16 transformed with pLH131 | this study |

DNA polymerase KOD for PCR reactions can be purchased from EMD Chemicals (San Diego, Calif.). All restriction enzymes and Antarctic phosphatase can be obtain from New England Biolabs (Ipswich, Mass.). Rapid DNA ligation kit is available from Roche (Manheim, Germany). Oligonucleotides can be ordered from Operon (Huntsville, Ala.S. All antibiotics and reagents in media are available from either Sigma Aldrich (St. Louis, Mo.) or Fisher Scientifics (Houston, Tex.).

Bacterial Strains.

Escherichia coli BW25113 (rrnB_{T14} ΔlacZ_{WJ16} hsdR514 ΔaraBAD_{AH33}ΔrhaBAD_{LD78}) was designated as the wild-type (WT) (Datsenko and Wanner, Proc. Natl. Acad. Sci. USA 97, 6640-6645, 2000) for comparison. In some experiments for isobutanol, JCL16 (rrnB_{T14} ΔlacZ_{WJ16} hsdR514ΔaraBAD_{AH33}ΔrhaBAD_{LD78}/F′ (traD36, proAB+, lacIq ZΔM15)) was used as wild-type (WT). Host gene deletions of metA, tdh, ilvB, ilvI, adhE, pta, ldhA, and pflB were achieved with P1 transduction using the Keio collection strains (Baba et al., Mol. Systems Biol. 2, 2006) as donor. The kan^{R} inserted into the target gene region was removed with pCP20 (Datsenko and Wanner, supra) in between each consecutive knock out. Then, removal of the gene segment was verified by colony PCR using the appropriate primers. XL-1 Blue (Stratagene, La Jolla, Calif.) was used to propagate all plasmids.

Plasmid Construction.

pSA40, pSA55, and pSA62 were designed and constructed as described elsewhere herein. The lacI gene was amplified with primers lacI SacI f and lacI SacI r from E. coli MG 1655 genomic DNA. The PCR product was then digested with SacI and ligated into the pSA55 open vector cut with the same enzyme behind the promoter of the ampicillin resistance gene, creating pSA55I.

The gene tdcB was amplified with PCR using primers tdcB f Acc65 and tdcB r SalI from the genomic DNA of E. coli BW25113 WT. The resulting PCR product was gel purified and digested with Acc65 and SalI. The digested fragment was then ligated into the pSA40 open vector cut with the same pair of enzymes, creating pCS14.

To replace the replication origin of pCS14 from colE1 to p15A, pZA31-luc was digested with SacI and AvrII. The shorter fragment was gel purified and cloned into plasmid pCS14 cut with the same enzymes, creating pCS16.

The operon leuABCD was amplified using primers A106 and A109 and E. coli BW25113 genomic DNA as the template. The PCR product was cut with SalI and BglII and ligated into pCS16 digested with SalI and BamHI, creating pCS20.

To create an expression plasmid identical to pSA40 but with p15A origin, the p15A fragment obtained from digesting pZA31-luc with SacI and AvrII was cloned into pSA40 open vector cut with the same restriction enzymes, creating pCS27.

The leuA* G462D mutant was constructed using SOE (Splice Overlap extension) with primers G462Df and G462Dr and the E. coli BW25113 WT genomic DNA as a template to obtain leuA*BCD. Then the SOE product was digested and cloned into the restriction sites Acc65 and XbaI to create PZE_leuABCD. The resulting plasmid was next used as a template to PCR out the leuA*BCD using primers A106 and A109. The product was cut with SalI and BglII and ligated into pCS27 digested with SalI and BamHI, creating pCS48.

The gene ilvA was amplified from E. coli BW25113 WT genomic DNA with primers A110 and A112. Next, it was cut with Acc65 and XhoI and ligated into the pCS48 open vector digested with Acc65 and SalI, creating pCS51.

The gene tdcB from the genomic DNA of E. coli BW25113 WT was amplified with PCR using primers tdcB f Acc65 and tdcB r SalI. The resulting PCR product was gel purified, digested with Acc65 and SalI and then ligated into the pCS48 open vector cut with the same pair of enzymes, creating pCS50.

WT thrABC was amplified by PCR using primers thrA f Acc65 and thrC r HindIII. The resulting product was digested with Acc65 and HindIII and cloned into pSA40 cut with the same pair of enzymes, creating pCS41.

To replace the replication origin of pCS41 from colE1 to pSC101, pZS24-MCS1 was digested with SacI and AvrII. The shorter fragment was gel purified and cloned into plasmid pCS41 cut with the same enzymes, creating pCS59.

The feedback resistant mutant thrA* was amplified by PCR along with thrB and thrC from the genomic DNA isolated from the threonine over-producer ATCC 21277 using primers thrA f Acc65 and thrC r HindIII. The resulting product was digested with Acc65 and HindIII and cloned into pSA40 cut with the same pair of enzymes, creating pCS43.

To replace the replication origin of pCS43 from colE1 to pSC101, pZS24-MCS1 was digested with SacI and AvrII. The shorter fragment was gel purified and cloned into plasmid pCS43 cut with the same enzymes, creating pCS49.

Branched-chain amino-acid aminotransferase (encoded by ilvE) and tyrosine aminotransferase (encoded by tyrB) were deleted by P1 transduction from strains disclosed in Baba et al.

To clone the L-valine biosynthesis genes i) ilvIHCD (EC) and ii) als (BS) along with ilvCD (EC), the low copy origin of replication (ori) from pZS24-MCS1 was removed by digestion with SacI and AvrII, and ligated into the corresponding sites of i) pSA54 and ii) pSA69 to create plasmid pIAA1 and pIAA11, respectively.

To clone kivd from L. lactis and ADH2 from S. cerevisiae, the ColE1 on of pSA55 was removed by digestion with SacI and AvrII and replaced with the p15A on of pSA54 digested with the same restriction enzymes to create pIAA13. To better control the expression of these genes, lacI was amplified from E. coli MG1655 genomic DNA with KOD polymerase using primers lacISaclf and lacISaclr and ligated into the SacI site of pCS22 to be expressed along with the ampicillin resistance gene, bla, and create plasmid pIAA12.

In order to overexpress the leuABCD operon in BW25113/F′ from the chromosome, the native promoter and leader sequence was replaced with the P_{LlacO-1} promoter. The P_{LlacO-1} promoter was amplified from pZE12-luc with KOD polymerase using primers lacO1KanSOEf and lacO1LeuA1r. The gene encoding resistance to kanamycin, aph, was amplified from pKD13 using primers KanLeuO1f and KanlacO1SOEr. 1 μL of product from each reaction was added as template along with primers KanLeuO2f and lacO1LeuA2r, and was amplified with KOD polymerase using SOE. The new construct was amplified from the genomic DNA of kanamycin resistant clones using primers leuKOv1 and leuKOv2 and sent out for sequence verification to confirm the accuracy of cloning. To overexpress the leuABCD operon from plasmid, the p15A on from pSA54 was removed with SacI and AvrII and ligated into the corresponding sites of pCS22 (ColE1, Cm^{R}, P_{LlacO-1}: leuABCD) to create plasmid pIAA2. In order for tighter expression, lad was amplified and ligated as described previously for pIAA12 into pCS22 to be expressed along with the chloroamphenicol resistance gene, cat, and create plasmid pIAA15. Plasmid pIAA16 containing leuA(G1385A) encoding for IPMS (G462D) was created by ligating the 5.5 kb fragment of pIAA15 digested with XhoI and NdeI and ligating it with the 2.3 kb fragment of pZE12-leuABCD (ColE1, Amp^{R}, P_{LlacO-1}: leuA(G1385A)BCD) cut with the same restriction enzymes. To control for expression level, the RBS was replaced in pIAA15 to match that of pIAA16. To do this, the 5.6 kb fragment of pIAA16 from digestion with HindIII and NdeI was ligated with the 2.2 kb fragment of pIAA15 digested with the same enzymes to create pIAA17.

Media and Cultivation.

Certain strains were grown in a modified M9 medium (6 g Na₂HPO4, 3 g KH₂PO₄, 1 g NH₄Cl, 0.5 g NaCl, 1 mM MgSO₄, 1 mM CaCl₂, 10 mg Vitamin B1 per liter of water) containing 10 g/L of glucose, 5 g/L of yeast extract, and 1000× Trace Metals Mix A5 (2.86 g H₃BO₃, 1.81 g MnCl₂.4H₂O, 0.222 g ZnSO₄.7H₂O, 0.39 g Na₂MoO₄.2H₂O, 0.079 g CuSO₄.5H₂O, 49.4 mg Co(NO₃)₂.6H₂O per liter water) inoculated 1% from 3 mL overnight cultures in LB into 10 mL of fresh media in 125 mL screw cap flasks and grown at 37° C. in a rotary shaker for 4 hours. The culture was then induced with 1 mM IPTG and grown at 30° C. for 18 hours. Antibiotics were added as needed (ampicillin 100 μg/mL, chloroamphenicol 35 μg/mL, kanamycin 50 μg/mL).

For some alcohol fermentation experiments, single colonies were picked from LB plates and inoculated into 3 ml of LB media with the appropriate antibiotics (ampicillin 100 μg/ml, kanamycin 50 μg/ml, and spectinomycin 50 μg/ml). The overnight culture grown in LB at 37° C. in a rotary shaker (250 rpm) was then inoculated (1% vol/vol) into 20 ml of M9 medium (6 g Na₂HPO₄, 3 g KH₂PO₄, 0.5 g NaCl, 1 g NH₄Cl, 1 mM MgSO₄, 10 mg vitamin B1 and 0.1 mM CaCl₂ per liter of water) containing 30 g/L glucose, 5 g/L yeast extract, appropriate antibiotics, and 1000× Trace Metal Mix A5 (2.86 g H₃BO₃, 1.81 g MnCl₂.4H₂O, 0.222 g ZnSO₄.7H₂O, 0.39 g Na₂MoO₄.2H₂O, 0.079 g CuSO₄.5H₂O, 49.4 mg Co(NO₃)₂.6H₂O per liter water) in 250 ml conical flask. The culture was allowed to grow at 37° C. in a rotary shaker (250 rpm) to an OD₆₀₀ of 0.4˜0.6, then 12 ml of the culture was transferred to a 250 ml screw capped conical flask and induced with 1 mM IPTG. The induced cultures were grown at 30° C. in a rotary shaker (240 rpm). Samples were taken throughout the next three to four days by opening the screwed caps of the flasks, and culture broths were either centrifuged or filtered to retrieve the supernatant. In some experiments as indicated, 8 g/L of threonine was added directly into the cell culture at the same time of induction.

α-keto acid experiments were done under oxygen ‘rich’ conditions unless otherwise noted. For oxygen rich experiments, 10 mL cultures in 250 mL baffled shake flasks were inoculated 1% from 3 mL overnight cultures in LB. For oxygen poor experiments, 10 mL cultures were inoculated in 125 mL screw caps. All cultures were grown at 37° C. for 4 hours and induced with 1 mM IPTG and harvested after 18 hrs of growth at 30° C.

Metabolite Detections.

The produced alcohol compounds can be quantified by a gas chromatograph (GC) equipped with flame ionization detector. The system includes model 5890A GC (Hewlett-Packard, Avondale, Pa.) and a model 7673A automatic injector, sampler and controller (Hewlett-Packard). Supernatant of culture broth (0.1 ml) is injected in split injection mode (1:15 split ratio) using methanol as the internal standard.

The separation of alcohol compounds is carried out by A DB-WAX capillary column (30 m, 0.32 mm-i.d., 0.50 μm-film thickness) purchased from Agilent Technologies (Santa Clara, Calif.). GC oven temperature is initially held at 40° C. for 5 min and raised with a gradient of 15° C./min until 120° C. It is then raised with a gradient of 50° C./min until 230° C. and held for 4 min. Helium is used as the carrier gas with 9.3 psi inlet pressure. The injector and detector are maintained at 225° C. 0.5 ul supernatant of culture broth is injected in split injection mode with a 1:15 split ratio. Methanol is used as the internal standard.

For other secreted metabolites, filtered supernatant is applied (20 ul) to an Agilent 1100 HPLC equipped with an auto-sampler (Agilent Technologies) and a BioRad (Biorad Laboratories, Hercules, Calif.) Aminex HPX87 column (5 mM H₂SO₄, 0.6 ml/min, column temperature at 65° C.). Glucose is detected with a refractive index detector, while organic acids are detected using a photodiode array detector at 210 nm. Concentrations are determined by extrapolation from standard curves.

For other secreted metabolites, filtered supernatant is applied (0.02 ml) to an Agilent 1100 HPLC equipped with an auto-sampler (Agilent Technologies) and a BioRad (Biorad Laboratories, Hercules, Calif.) Aminex HPX87 column (0.5 mM H2SO4, 0.6 mL/min, column temperature at 65° C.). Glucose is detected with a refractive index detector while organic acids are detected using a photodiode array detector at 210 nm. Concentrations are determined by extrapolation from standard curves.

Cyanobacteria encompass a large group of photosynthetic microorganisms that vary widely in morphology, habitat, and physiology. Included in this group is the unicellular Synechococcus sp. strain PCC 7942 (previously Anacystis nidulans R2), which is one of the few cyanobacterial strains which have been well-characterized in terms of physiology, biochemistry, and genetics. As stated previously, S. elongatus PCC7942 has been engineered to produce up to 1.1 g/L of isobutryaldehyde from CO₂ (see, e.g., Atsumi et al., 2009) by utilizing the microorganism's photosynthesis and CBB cycle. In addition to S. elongatus PCC7942, other cyanobacterial strains can be used. For example, S. elongatus PCC7002 has the ability to grow heterotrophically on glycerol and has a shorter generation time of 4 hr compared to 6.4 hr for S. elongatus PCC7942.

In order to engineer S. elongatus to utilize H₂ as an electron donor, strains that express hydrogenase genes from Ra. eutropha, B. japonicum, R. capsulatus, and Rh. palustris are constructed by chromosomal insertion of the expression cassettes into neutral site 1 (NSI). An expression cassette is thus created by cloning the individual genes into the NSI-targeting vector, pAM2991 under the IPTG-inducible Ptrc promoter. Methods for measuring in vitro and in vivo hydrogenase activity have been well-established (Vignais and Billoud, 2007) and can be used to determine the best hydrogenase for a particular system.

To improve the H₂ uptake rate of the hydrogenases error prone PCR can be used on one of the oxygen-tolerant hydrogenases (e.g., from Ra. eutropha). Under conditions where the photosynthetic activity of Synechococcus is relatively low (i.e., low light conditions), the fastest growing transformants can be analyzed for improvements in H₂ uptake (Vignais and Billoud, 2007). Other approaches can be used to capitalize on the loss of autotrophic growth, but maintenance of heterotrophic growth of a Ra. eutropha ΔhoxFUYG hydrogenase mutant (Massanz, 1998). An expression library of mutant, oxygen-tolerant hydrogenases created by error-prone PCR from Ra. eutropha and/or other species will be transformed into the Ra. eutropha ΔhoxFUYG hydrogenase mutant. Grown under lithoautotrophic conditions, the fastest growing transformants express mutant hydrogenases with improved H₂ uptake and/or activity, which can be ascertained by H₂ uptake assays (Vignais and Billoud, 2007). The genes that express these mutant hydrogenases with improved H₂ uptake activity can be cloned into the NSI-targeting vector and introduced into S. elongatus for expression.

In order to engineer S. elongatus to oxidize formate for the production of reducing equivalents, formate dehydrogenases (FDHs) are heterologously expressed in this microorganism. FDHs have been proven to be the most promising candidate for the development of NAD+ regeneration systems in organic synthesis for production of high-added-value products largely due to their wide pH-optimum (pH 6.0-9.0) and to the nonreversibility of enzymes (Burton, 2003; Hummel and Kula, 1989; Shaked et al., 1980; Wichmann and Vasic-Racki, 2005). Of the FDHs that have been studied, the one from Candida boidinii is the most commonly used for the development of NAD+ regeneration systems (Ohshima et al., 1985). Studies on C. boidinii FDH have identified mutations that confer altered cofactor specificity (Rozzell, 2004), improved catalytic activity (Slusarczyk, 2003), and enhanced chemical stability (Slusarczyk, 2003; Felber, 2001). Using various optimized FDH, the activity in S. elongates can be optimized, especially in altering the cofactor specificity from NAD(H) to NADP(H) because S. elongatus has a preference for NADP(H) (Tamoi et al., 2005).

Several FDHs have been integrated into the NSI site of S. elongatus PCC7942. The genes that encode the wild type and D195S/Y196H double mutant FDH from C. boidinii and the FDH from M. thermoacetica were each cloned into the NSI-targeting vector, under the IPTG-inducible Ptrc promoter. The D195S/Y196H double mutation was utilized because it results in a FDH with altered cofactor specificity from NAD(H) to NADP(H). The FDH gene from Moorella thermoacetica, encoded by Moth_{—}2314, has been indicated to encode for an enzyme with formate:NADP+ oxidoreductase activity. This enzyme was chosen because of its cofactor preference.

In addition to the FDHs, other genes were also heterologously expressed to optimize formate utilization. To ensure efficient formate uptake, a formate transporter encoded by focA from E. coli was also overexpressed. Furthermore, to specifically generate NADPH from formate oxidization, several transhydrogenases including pntAB and udhA from E. coli have been introduced in combination with wild type NAD+-dependent C. boidinii FDH. By using enzymatic assays of crude cyanobacterial cell lysates, as well as HPLC measurements of formate consumption in flask culture, the co-expression of E. coli focA, C. boidinii wild type FDH, and E. coli pntAB enable S. elongatus to consume formate at a significant rate.

To improve CO₂ fixation, an additional copy of the CBB cycle genes, rbcLS, were integrated into the chromosome of the isobutyraldehyde S. elongatus PCC7942 production strain, resulting in a 2-fold increase in isobutyraldehyde (Atsumi et al., 2009). This example, along with successful examples of fructose-1,6/sedoheptulose-1,7-bisphosphatase overexpression (Miyagawa et al. 2001; Ma et al. 2005), illustrate that overexpression of CBB enzymes can enhance photosynthesis efficiency, growth characteristics, and biofuel production. Additional copies of many of the CBB cycle genes have been integrated into the NSI and NSII sites of S. elongatus PCC7942. Genes that have been integrated include those that encode for fructose-1-6-bisphosphatase 1 (Synpcc7942_{—}2335), ribulose-phosphate 3-epimerase (Synpcc7942_{—}0604), sedoheptulose bisphosphatase (Synpcc7942_{—}0505), ribose 5-phosphate isomerase (Synpcc7942_{—}0584), phosphoribulokinase (Synpcc7942_{—}0977), and the E. coli transketolase, tktA.

In cyanobacteria and higher plants, CO₂ fixation is regulated by various regulation pathways, which can be divided into two major categories: transcriptional and posttranslational. In both cases, the redox status of the photosynthetic electron transportation chain has been proposed to play an important role in light sensing as the signaling input pathway (Buchanan and Balmer, 2005; Golden, 1995). Once received, the light signal is then relayed from the photosynthetic machinery to other cellular mediators, including various proteins in the ferredoxin/thioredoxin system and KaiABC oscillator system (Buchanan and Balmer, 2005; Ivleva et al., 2006; Lindahl and Florencio, 2003; Schmitz et al., 2000).

Transcription of most of the CBB cycle genes are significantly suppressed in the dark cycle (Ito et al., 2009; Nakahira et al., 2004). One of the most extensively studied regulation systems in S. elongatus PCC7942 is the KaiABC circadian rhythm oscillator system, which governs the global transcription profile in a diurnal cyclic fashion (Ishiura et al., 1998; Johnson et al., 2008). Recent studies have shown that transcriptional activity from most of the promoters in S. elongatus displayed substantial fluctuation over a day/night cycle (Ito et al., 2009; Liu et al., 1995; Smith and Williams, 2006). Moreover, the overall organization of the S. elongatus chromosome undergoes cyclic change (Nakahira et al., 2004; Smith and Williams, 2006), which may affect the expression level of both endogenous and genome-integrated heterogeneous production pathways. Previous studies have shown that disruption of the kaiABC gene cluster delivered the arrhythmia phenotype in S. elongatus PCC7942, although the average expression level of each individual gene in the genome was not dramatically altered (Ito et al., 2009). This and similar arrhythmic strains may be favored for CO₂ fixation in the dark, due to their steady global gene expression levels regardless of changing light condition. In addition, to maintain CBB gene expression at a high level, enzymes such as RuBisCO, phosphoribulokinase (PRK), and glyceraldehyde-3-phosphate dehydrogenase (GAPDH) can be constitutively overexpressed.

Posttranslational level (or protein level) regulation represents another layer of light/dark regulation of CO₂ fixation on top of transcriptional regulation. The exchange of dithiol/disulfide status controlled by the ferredoxin/thioredoxin system is one of these conserved posttranslational regulation mechanism utilized by chloroplasts of plants, algae, as well as photosynthetic microorganisms, to adjust enzyme activities according to light condition (Buchanan et al., 1980; Pfannschmidt et al., 2000; Buchanan et al., 2002; Lindahl et al., 2003). In light conditions, ferredoxin receives electrons from Photosystem I (PS I) and transfers them to thioredoxin (Trx), mediated by the enzyme ferredoxin-Trx reductase (FTR). Thioredoxin can then reduce disulfide bonds formed between cysteine residues within target enzymes and thus modulate their activities.

In contrast to higher plants, most enzymes in the CBB cycle of cyanobacterium Synechocystis sp. PCC 6803 are not directly regulated by the ferredoxin/thioredoxin system (Lindahl and Florencio, 2003). Specifically, although fructose-1,6-bisphosphatase (FBPase), NADP+-glycerolaldehyde-3-phosphate dehydrogenase (NADP+-GAPDH), and phosphoribulokinase (PRK) are greatly suppressed in the dark condition by redox regulation in higher plants (Buchanan, 1980), similar redox regulation of these three enzymes have been suggested to be absent in cyanobacteria Synechocystis sp. PCC 6803 and Synechococcus elongatus PCC7942 by biochemical studies (Tamoi et al., 1996; Tamoi et al., 1998). Consistently, it has also been indicated from amino acid sequence alignment that the potential regulatory cysteine residues are missing in cyanobacterial NADP+-GAPDH and FBPase (Tamoi et al., 1996; Tamoi et al., 1998).

Thus, removing ferredoxin/thioredoxin-mediated redox regulation of the CBB enzymes in cyanobacteria can be performed. RuBisCO has been suggested to be a conserved ferredoxin/thioredoxin target (Lindahl and Florencio, 2003). Fortunately, with a C172A mutation in the RuBisCO of Synechocystis sp. strain PCC6803, the inhibitory effect of oxidants that react with the vicinal thiols in RuBisCO is alleviated (Marcus et al., 2003). Since the regulatory cysteines are conserved among cyanobacteria species, these observations provided useful information for protein engineering in the construction of a redox-resistant RuBisCO in S. elongatus PCC7942.

Besides the universal redox regulation system shared by all photosynthetic organisms, cyanobacterial cells also possess other unique posttranslational mechanisms to regulate CO₂ fixation. For example, protein CP12 in S. elongatus PCC7942 has been found to form a complex with RuBisCO and GAPDH to inhibit their activities in the dark (Wedel and Soll, 1998). Furthermore, the formation of this complex is dynamically regulated by CP12, which is able to sense the NAD(H)/NADP(H) ratio under light/dark conditions (Tamoi et al., 2005). In cyanobacteria, mutations that prevent CP12 expression had no effect during conditions of continuous light, but resulted in inhibited growth in light/dark diurnal conditions presumably due to a carbon metabolism disorder related to leaky CBB cycle activity in the dark (Tamoi et al., 2005). By inactivating CP12 using genetic or protein engineering approaches, formation of the inhibitory complex could be eliminated, releasing the CBB cycle from light/dark regulation.

As a chemolithoautotroph, Ra. eutropha is able to derive its energy and reducing power from inorganic compounds or elements, such as H₂ or formate, to drive CO₂ fixation through the CBB cycle.

Ra. eutropha employs native hydrogen utilization pathways when it undergoes chemoautotrophic growth. Two types of hydrogen utilization pathways run in parallel to fuel the CO₂-fixing CBB cycle with ATP and NADPH: A membrane-bound hydrogenase (MBH), which oxidizes H₂ and feeds electrons into the respiratory chain to generate ATP; and also a soluble hydrogenase (SH), which directly uses NAD(P)+ as an electron acceptor to produce NAD(P)H at the expense of H₂. In addition, several transhydrogenases convert NADH into NADPH in order to meet the NADPH needs required by the CBB cycle (Cramm, 2009; Pohlmann et al., 2006). Ra. eutropha hydrogenases belong to a family of (NiFe) bidirectional hydrogenases. However, unlike most of the members in the family, which are sensitive to very low oxygen concentrations, Ra. eutropha hydrogenases are relatively oxygen tolerant, consistent with the aerobic physiological nature of this organism.

Similarly, formate can serve as both an electron donor and carbon source to sustain autotrophic growth of Ra. eutropha. A membrane-bound formate dehydrogenase oxidizes formate and transports the electrons into respiratory chain; and a soluble formate dehydrogenase uses NAD+ as the electron acceptor. The CO₂ produced from formate oxidization is then assimilated (Cramm, 2009; Pohlmann et al., 2006).

CO₂ is fixed through the CBB cycle in Ra. eutropha to pyruvate. By engineering alsS from B. subtilis, ilvCD and yqhD from E. coli, and kivd from L. lactis into Ra. eutropha autotrophic isobutanol synthesis can be obtained.

To enhance isobutanol production efficiency, competing pathways that dissipate reducing equivalence or drain carbon flux can be eliminated. In Ra. eutropha, a prominent example would be the PHA production pathway. The cells can naturally accumulate up to about 70% PHA (of the cell mass), even in autotrophic conditions with CO₂ and H₂ as substrates (Tanaka et al., 1995), which utilizes a large portion of carbon source and NADPH pools. Fortunately, the PHA production pathway is very well known and genetic manipulation tools to perform knock-out studies are available.

To achieve high titer levels of isobutanol production, it is beneficial to isolate a mutant that has a higher tolerance to isobutanol. The gram-negative Ra. eutropha appears to have comparable solvent tolerance to that of E. coli. Given the success in developing and characterizing E. coli strains that can tolerate up to 8 g/L isobutanol, similar mutagenesis approaches can be utilized in addition to solvent challenging selection. Furthermore, based on high-throughput genomic DNA sequencing of the solvent tolerant strains generated by our group as well as others, rational strain engineering approaches may also become available.

Purple bacteria, such as Rhodopsudomonas and Rhodobacter, demonstrate lithoautotrophic and chemoautotrophic growth with many organic and inorganic electron donors, including hydrogen and formate. These microorganisms are able to grow in a mineral medium in the dark at the expense of hydrogen, oxygen, and CO₂. Although their growth is sensitive to O₂, the presence of methanol in the medium can improve oxygen tolerance (Siefert and Pfennig, 1979). Given these factorable characteristics Rh. palustris can be a host for isobutanol synthesis from CO₂ and H₂ or formate.

Either co-replicated plasmids or chromosome integration is used to express enzymes of the isobutanol pathway. Specifically, alsS from B. subtilis, ilvCD and yqhD from E. coli, and kivd and yqhD from L. lactis can be engineered into the microorganism. Functional expression of the pathway can be examined by enzyme assays and by measuring the production of isobutanol under chemoheterotrophic growth conditions. Isobutanol production in Rh. palustris can be investigated in electron-autotrophic conditions with hydrogen or formate as the electron donor. Electron-autotrophic biofuel production is performed in the dark under either aerobic or microaerobic conditions.

Rh. palustris is able to sense redox status and ATP levels, and is thus able to change metabolic modes according to changes in culture conditions (Larimer et al., 2004). Experimental evidence has shown that single-gene deletions of cbbRRS results in a significant reduction in total RuBisCO activity, which indicates that the cbbRRS is essential for RuBisCO expression (Romagnoli and Tabita, 2006). Therefore, in order to improve or maintain CBB cycle activity during different metabolic conditions, upregulation of cbbRRS by overexpression or modify the PAS domains of cbbR can be performed to make it more efficient in catalyzing the phosphorylation cascade.

To select host organisms for further development the host strain will be exposed to mutagens, and then the surviving culture will be enriched for chemoautotrophic growth. Through several generation of metabolic evolution, the fast-growing mutants will dominate the culture. Since fast growth indicates high carbon fixation rates, these mutants most likely will demonstrate improved CBB pathway efficiency and will be subject to further engineering, such as deregulation and overexpression of CBB pathway enzymes.

In addition, the metabolite profile of electron-autotrophic production conditions is analyzed with HPLC-DAD and GC-FID. Once the major by-products are confirmed, the critical genes that are responsible for their formation are identified for inactivation. The isobutanol production efficiency is also controlled by the reducing power supply. Overexpression of NAD(P)H-generating hydrogenases and formate hydrogenases can improve energy input and biofuel production efficiency in the system.

H₂ can be produced by the electrolysis of water. In conventional electrolyzers, 25˜30% potassium hydroxide is added to facilitate the dissociation of water into H⁺ and OH⁻. It is however corrosive to operate electrolysis in a basic environment. As a result, solid polymer electrolyte membranes (SPE) or proton exchange membranes (PEM) were developed to aid in the splitting of water in a neutral environment. The SPE or PEM electrolyzer, as the name implies, contains a polymer as a membrane separating the cathode side from the anode side. The formation of O₂ and H₂ is separated into two compartments by a solid electrolyte membrane. One of the most commonly used solid electrolytes is nafion. The solvated SO³⁻ ions act as the proton carriers, which carries protons from the anode to the cathode, which is later reduced to H₂. The efficiency of the SPE membrane electrolyzer is estimated to be about 80˜94%.

The electro-autotrophic fermentation system uses gas-phase substrates to supply for carbon and reducing power needs. When the gases are fed into the bioreactor, the solubility of the gases will normally be very low. Fortunately, the electro-autotrophic organisms of the disclosure have lower metabolic activities compared to conventional sugar-based fermentations. In order to minimize energy consumption, impellers are avoided which are energy intensive. Instead, mass transfer and cell suspension will be used to optimize the gas circulation rate. The gas stream is replenished and recycled to complete a closed system with no H₂ outlet. In addition, the ratio of the three components (H₂, O₂, and CO₂) is optimized for growth and productivity. Optimization of pH, temperature, medium components (among others) is also performed and is within the skill in the art.

For isobutanol purification, several conventional n-butanol separation technologies are known (e.g., gas-stripping and adsorption).

To develop Ralstonia eutropha as an isobutanol producer the valine biosynthetic pathway was strengthened to make enough 2-KIV (2-ketoisovalerate), which is the precursor for isobutanol. The synthetic pathway genes to convert 2-KIV into isobutanol were then engineered into the microorganism.

Since isobutanol is produced by decarboxylation and subsequent reduction of 2-Ketoisovalerate (2-KIV), an intermediate in valine biosynthesis, it is essential to enhance metabolic flux through valine biosynthesis pathway in the host. One approach taken was to strengthen natural valine biosynthetic pathway in Ralstonia, while a second approach taken was to introduce heterologous genes for valine biosynthesis pathway. In the genome of Ralstonia eutropha, the naturally existing 2-KIV biosynthesis pathway genes include ilvBHC and ilvD genes at separate loci. These natural genes were overexpressed within Ralstonia eutropha by chromosomal knocking-in of a strong phaC promoter in front of the corresponding operons. Another approach introduced foreign genes for valine biosynthesis pathway. In the second method the artificial operon of alsS from B. subtilis and ilvCD from Escherichia coli was used under the phaC promoter of Ralstonia eutropha. This artificial operon was introduced into chromosomal phaB2-phaC2 loci by conjugational double-crossover integration.

To verify the enhanced activities of 2-KIV production enzymes, the enzyme activities of these 3 enzymes was analyzed. Compared to wild type Ralstonia eutropha strain H16, cells (LH66) with modifications in natural valine biosynthesis genes using the phaC promoter showed around 9 fold, 3 fold, and 4 fold increase of ilvBH, ilvC, ilvD activities, respectively. The alsS gene from Bacillus subtilis have higher catalytic activity and affinity to pyruvate and were expected to be more productive. As expected the strain (LH67), which has an integrated artificial operon of alsS from B. subtilis and ilvCD from Escherichia coli driven by phaC promoter in the genome, showed much better enzyme activities in all three enzymes. Therefore, this LH67 strain was used for the construction of isobutanol production strain in Ralstonia eutropha.

For the efficient conversion of 2-KIV into isobutanol, two more enzymatic reactions catalyzed by a 2-keto acid decarboxylase (KDC) and an alcohol dehydrogenase (ADH) were used. kivd from Lactococcus lactis was selected as the KDC for its high specificity towards 2-KIV and Adh2 from Saccharomyces cerevisiae and yqhD from E. coli were both tested as the ADH candidates for their different preference to cofactors NADH and NADPH, respectively. A plasmid containing kivd and either Adh2 or yqhD was transformed into Ralstonia cells and tested for activity to convert 2-KIV into isobutanol. Although the cells with kivd and Adh2 produced isobutanol from 2-KIV, the yqhD was a better alcohol dehydrogenase in Ralstonia to produce isobutanol efficiently. Based on these result, yqhD was shown to be more active for reducing isobutyaldehyde to isobutanol, because of the higher intracellular NADPH level than NADH in the Ralstonia eutropha.

Using these two genes (kivd, yqhD), 5 different configurations were constructed for the expression of kivd and yqhD, either chromosomal or plasmid. After construction of strains, the efficiency of these enzymes expressed in Ralstonia were measured by feeding experiment of 2-KIV. After 24 hr, the isobutanol production from 2-KIV was measured from these strains. The kivd-yqhD operons driven by CAT gene promoter and phaP promoter were successful in converting 2-KIV into isobutanol. The plasmid harboring Pcat promoter version of kivd-yqhD operon was used for the construction of isobutanol production strain.

After construction of all the functionally expressed 5 genes needed for the production of isobutanol from pyruvate, the various enzymes and operons were engineered into one organism to construct an isobutanol producing Ralstonia eutropha strain. LH67, which showed the strongest enzyme activities for alsS and ilvCD, was transformed with the plasmid harboring the most efficient kivd-yqhD operon with Pcat promoter. The final strain, LH74, was tested for the production of isobutanol. In 5 L fermentor operation, this strain was found to produce 120 mg/L of isobutanol from fructose as carbon source in 40 hours. Interestingly, this strain also produced 180 mg/L of 3-Methyl-1-butanol, which is also good higher alcohol biofuel.

To test the electro-autotrophic production of isobutanol by R. eutropha strain LH74, the strain was cultured in minimal media using 5 L fermentor with autotrophic gas mixing condition (hydrogen, carbon dioxide, and oxygen=10:1:1). Carbon dioxide is the only carbon source provided in this fermentation. All gases were bubbled into the fermentor under atmospheric pressure and the pH of the culture was held constant at 7.0. The produced higher alcohols were collected using chilled condensing system from vent-gas line of fermentor. This fermentation was run over a 5.8 day period and produced a total 67.7 mg/L of isobutanol with a final OD₆₀₀ₙₘ of 12.72 (OD₄₃₆ₙₘ higher than 20). Both the OD and the isobutanol production continued to climb over the duration of the 5.8 day fermentation. The isobutanol production showed no signs of a plateau after 5.8 days. However, under these conditions, major carbon flow from CO₂ fixation via CBB pathway is still directed toward cell mass production rather than biofuel production. This experiment demonstrates isobutanol production in autotrophic conditions using R. eutropha indicating successful electro-autotrophic production of higher alcohol.

From the intermediate 2-Ketoisovalerate (2-KIV) feeding experiment, the data suggested that the activity of the keto acid decarboxylation and reduction part of the pathway (catalyzed by kivd and yqhD) may not be the limiting factor of the production rate in vivo. Therefore, one of the hypotheses could be that the part of the pathway upstream of kivd and yqhD may be the bottleneck of isobutanol production in this strain. This part of the pathway overlaps with the native valine biosynthesis pathway and was enhanced by overexpressing alsS (Bacillus subtilis), ilvC (Escherichia coli), and ilvD (Escherichia coli). Although the activities of alsS, ilvC, and ilvD were measured in enzymatic assays and shown significant increased compared to wildtype strain, the absolute value of the enzymatic activity was lower than E. coli isobutanol production strains in other research. And because the alsS, ilvC, and ilvD operon was integrated into the Ralstonia chromosome with only one copy (LH74), it was reasoned that the relatively low activity of this part of the pathway may be due to the low gene dosage in the strain.

To explore this possibility, alsS, ilvC, and ilvD were also put into a multiple copy plasmid in addition to kivd and yqhD. The whole operon containing all five genes of the pathway was driven by the pPhaP promoter. After transforming this plasmid into wildtype Ralstonia cells, the resulted strain was able to produce around 200 mg/L isobutanol in one day in minimal medium with fructose as the substrate, which is over two fold of the amount produced by the previous strain in the same condition. The final titer of isobutanol can reach around 500 mg/L in minimal medium with fructose, although in these experiments the cell growth was retarded and the production limited after two days, indicating toxicity of the production pathway caused by the high level overexpression from the multiple copy plasmid.

To overcome the toxicity effect while still maintaining the high gene dosage conveyed by the plasmid system, the alsS from Bacillus subtilis is replaced by several acetohydroxy acid synthase (AHAS) genes from different organisms in the multiple copy plasmids and tested for the activity and toxicity. The genes tested include ilvBN (E. coli), ilvIH (E. coli), and alsS (Klebsiella pneumoniae). The results showed that different AHAS proteins may have a broad range of activity in vivo, resulting in different isobutanol production rate and titer. For example, when alsS from Klebsiella pneumoniae is overexpressed, the cells were able to produce around 1.2 g/L isobutanol in minimal medium with fructose in one day. However, although the AHASs tested vary in protein sequences and structures, all of them resulted in toxicity, indicating the toxicity of the pathway may not be due to the protein expression or folding problem related to one specific AHAS protein.

For electro-produced formate as a single carbon source, conditions for autotrophic growth on formate were developed. Under standard minimal medium (German medium) with formate, Ralstonia showed very poor growth. To overcome this buffered medium with HEPES was used to control pH during growth. Using this growth condition, more than OD₄₃₆ₙₘ 1 was grown in 2 days.

The genes kivd (Lactococcus lactis) and yqhD (E. coli) were introduced by a multiple-copy plasmid. The genes were amplified using genomic DNA of appropriate organisms. The yqhD gene was chosen as the alcohol dehydrogenase because it is NADPH dependent. The highly active polyhydroxyalkanoate (PHA) production pathway in this organism uses NADPH as the reducing cofactor, suggesting that there is an abundant NADPH supply in the cell. In the lithoautotrophic biofuel production scenario, the oxidation of H₂ or formate directly yields NADH. But R. eutropha is equipped with an unusually high number of transhydrogenase isoenzymes that convert NADH to NADPH. Indeed, previous studies have shown that NADPH/NADP+ ratio is much higher than that of NADH/NAD+ under autotrophic condition, suggesting that the NADPH-dependent aldehyde reduction catalyzed by YqhD may also be favorable for biofuel production from CO₂. The kivd-yqhD artificial operon was then made by SOE PCR with the ribosome binding site sequence in front of each gene. The operon was assembled with the backbone of the broad-host-range vector pBHR1 (MoBiTec, Göttingen, Germany) using isothermal DNA assembly methods to form plasmid YL22 (Table 6). The kivd-yqhD operon was placed between the BspEI and NcoI restriction sites to disrupt the CAT gene in the plasmid. The promoter of the original CAT gene drives the expression of kivd-yqhD operon. The plasmid was then used to transform LH67 strain by electroporation. Briefly, overnight culture of R. eutropha in rich medium (16 g/L nutrient broth, 10 g/L Yeast extract, 5 g/L (NH4)SO4) was inoculated into 20 ml rich medium and allowed to grow to OD600=0.8 in 30° C.

The cells were harvested by centrifugation, washed twice with ice-cold 0.3M sucrose solution, and then resuspended in 2 ml of ice-cold 0.3M sucrose solution. 0.1 ml of this resuspended cells were mixed with ˜50 ng plasmid DNA and electroporated with 11.5 kV/cm, 5.0 ms, followed by rescuing with 0.2 ml rich medium in 30° C. for 2 hours and plated on rich medium plates containing 200 mg/l kanamycin. Colonies from the transformation were confirmed by PCR. The strain was named LH74D (Table 6).

### Construction of the lacZ Bearing Ralstonia Strains

The DNA fragments from −500 bp to +150 bp relative to the katG, sodC, and norA gene open reading-frame start codon of R. eutropha were amplified from the genomic DNA and assembled with the lacZ gene (encoding the β-galactosidase) using SOE-PCR. The resulting products were then inserted between the BspEI and NcoI sites of broad-host-range vector pBHR1 using the isothermal DNA assembly method. The lacZ-gene cassette was placed in the opposite direction of the original CAT gene of the plasmid, to prevent the original CAT promoter from affecting lacZ transcription. The plasmids containing PkatG::lacZ, PnorA::lacZ, PsodC::lacZ were named pLH129, pLH130, pLH131, respectively (Table 6). R. eutropha H16 strain transformed with plasmids pLH129, pLH130, pLH131 by electroporation were named LH118, LH119, LH120, respectively (Table 6).

### Enzyme Assays

R. eutropha cells were cultured under autotrophic condition with H2:CO2:O2=8:1:1 in minimal medium for 48 hours in 30° C. 20 ml of culture was harvested by centrifugation, washed twice with ice-cold lysis buffer (5 mM MgSO4, 50 mM Tris-Cl, pH 8.0), and resuspended with 1 ml lysis buffer. After bead beating, the lysate was then centrifuged at 13,200 rpm for 20 minutes at 4° C. The supernatant was then retrieved for subsequent enzyme assays. Acetohydroxy-acid synthase (AHAS), ilvC, and ilvD assays were performed.

The β-galactosidase assays were performed as follows: After incubated overnight in rich medium (10 g/l peptone, 10 g/l yeast extract, 5 g/l beef extract, and 5 g/l (NH₄)₂SO₄), Ralstonia cells were harvest and inoculate into the electro-microbial bioreactors with 300 mL German minimal medium supplemented with 10 g/L Na₂SO₄ and 4 g/L fructose. Gas flow rate for the bioreactors was 200 mL/min for air and 30-40 mL/min for CO₂. Electrolysis was performed using a platinum mesh as the anode and an Indium foil as the cathode. Electricity was provided by the DC power supply. The voltage between two electrodes was around 4V and current was around 250 mA. For the control, no electrolysis was performed. After 3 hours, cells were harvested and concentrated by 100 fold. The reactions were started by adding appropriate amount of cells into a reaction mixture containing 100 ul chloroform, 50 ul 0.1% SDS, 200 ul ONPG (4 μg/ml), 950 ul Z buffer (Z buffer per 50 mL: 0.80 g Na2HPO4.7H2O, 0.28 g NaH₂PO₄.H₂O, 0.5 mL 1M KCl, 0.05 mL 1M MgSO₄, 0.135 mL β-mercaptoethanol). Vortex tubes for 10-15 sec. The assay proceeded for appropriate time. The assay was stopped by addition of 500 ul Na₂CO₃. Tubes were centrifuged at max speed for 1 min to separate chloroform. The aqueous layer was removed and the sample measured at A₄₂₀ (or A₄₀₅ for non-ideal case) and A₅₅₀. The amount of B-gal was calculated as follows:

B-gal units (Miller)=1000*(A₄₂₀−1.75*A₅₅₀)/(time*vol*OD600)
Miller units are in ΔA420 min-1 ml⁻¹.

### Conditions of Keto-Acid Feeding Experiments

R. eutropha cells were cultured in 20 ml minimal medium{containing 5 g/L fructose in 250 ml screw-cap shake flasks. When cell density reached OD₆₀₀₌0.3, 3 g/L 2-ketoisovalerate (KIV) was added. After 48 hours of incubation at 30° C., isobutanol and isobutyraldehyde were quantified using gas chromatography (GC).

### Heterotrophic Production Conditions

R. eutropha cells were cultured in German minimal medium containing 4 g/L fructose for 48 hours. Appropriate amount of cells were then washed and inoculated in 20 ml of the same medium in 250 ml screw-cap shake flasks to obtain initial OD₆₀₀ of 0.3. After 48 hours of incubation at 30° C., alcohols were quantified using gas chromatography (GC). Autotrophic production conditions R. eutropha cells were cultured in German minimal medium with the volume of 1.8 L in a 5 L fermentor with the gas flow rates were as follows: H₂ 200 mL/min, O₂/CO₂ mixture (1:1 ratio) 50 mL/min. The initial OD₆₀₀ was around 1.0. H₂ was provided by an electricity-powered hydrogen generator (No-Maintenance H₂ Generator 500, PerkinElmer Inc., CA) and fed directly to the fermentor without purification or compression. Evaporated alcohols in venting gas were condensed with a Graham condenser and collected. Daily, samples of culture broth and condensation liquid were taken and alcohols were quantified using gas chromatography (GC).

For the formate-based fermentation, R. eutropha LH74D cells were cultured in J minimal medium with the volume of 1.8 L in a 5 L fermentor. J minimal medium was prepared by autoclaving 1 g/L (NH₄)₂SO₄, 0.5 g/L KH₂PO₄, and 6.8 g/L NaHPO₄ in MilliQ ddH₂O and aseptically adding 0.2 g/L MgSO₄-7H₂O, 20 mg/L FeSO₄-7H₂O, 4 mg/L CaSO4-2H2O, 100 ug/L thiamine hydrochloride, and 1 ml/L SL7 metals solution (1% v/v 5M HCl (aq), 1.5 g/L FeCl₂-4H₂O, 0.19 g/L CoCl₂-6H₂O, 0.1 g/L MnCl₂-4H₂O, 0.07 g/L ZnCl₂, 0.062 g/L H₃BO₃, 0.036 g/L Na₂MoO₄-2H₂O, 0.025 g/L NiCl₂-6H₂O, and 0.017 g/L CuCl₂-2H₂O). Control set points for agitation, temperature, pH, DO, air flow % and O₂ flow % were 300 rpm, 300 C, 7.2, 5%, 100%, and 0%, respectively. Gas flow was controlled by a dynamic-control cascade driven by DO with a gas flow of 0.5 SLPM at 0% out and 2.5 SLPM at 100% out. To control pH, 50% v/v formic acid with 2 g/l KH₂PO₄ was fed in following a pH-driven control cascade set to no flow with 0% out and 1 second pulses every 10 seconds at −100% out by the controller. This feed thereby serves to lower the pH and replenish the carbon supply as formate is consumed by the cells. Evaporated alcohols in venting gas were condensed with a Graham condenser and collected. Samples of culture broth and condensation liquid were taken and alcohols were quantified using gas chromatography (GC). Under these conditions, the final titer was over 1.4 g/l (˜846 mg/l isobutanol and ˜570 mg/l 3MB) (FIG. 3C) and the peak productivity was around 25 mg/l/h.

Integrated electro-microbial fuel production was performed as follows: Ralstonia cells were inoculate into the electro-microbial bioreactors with 350 mL German minimal medium supplemented with 10 g/L Na₂SO₄. Gas flow rate for the bioreactors was 200 mL/min for air and 30-40 mL/min for CO₂. Electrolysis was performed using a platinum mesh as the anode and an Indium foil as the cathode. A porous ceramic cup was used to separate the cathode and the anode. Electricity was provided by the DC power supply. The voltage between two electrodes was around 4V and current was around 250 mA. Evaporated alcohols in venting gas were condensed with a Graham condenser and collected. Samples of culture broth and condensation liquid were taken and alcohols were quantified using gas chromatography (GC).

### Calculation of Formate or H₂-to-Isobutanol Energy Efficiency

The maximum efficiencies for the production of isobutanol and 3-methyl-butanol while using hydrogen as sole source of energy were calculated. Each problem was defined as the optimization of the respective product flux while constrained to a mass balance and a given input flux; this is described by:

Min(f^{T}ᵥ) such that Sᵥ=0 and v^{H}₂=1

Here S is the stoichiometric matrix of the system, is the vector of fluxes through each reaction in the system, and v is a vector such that fTv is the objective function. In the calculation, the system is defined by the reactions in the Calvin-Benson cycle, the reactions involved in glycolysis, the reactions in the valine and leucine biosynthesis pathway, the alcohol production reactions (KDC and ADH), H2 and CO2 import reactions, the alcohol outlet reactions and a reaction through which ATP is obtained through the oxidation of NAD(P)H (this reaction can have varying stoichiometry or P/O ratio ranging from 1.5 to 3.0). Additionally, the elements of vector f were set to zero for all elements except those corresponding to the flux of the alcohol being optimized (set to −1). Performing the optimization as described maximizes the amount of product obtained from 1 mole of formate or H2; the amount of formate or H2 needed to obtain 1 mole of product is therefore given by v-1alcohol.

The results were summarized as follows:

TABLE 7
Summary of theoretical yeild for higher alcohol production form formate
or H₂

Formate or H₂ needed to
produce 1 mole alcohol (mole)

| P/O ratio (ATP/NAD(P)H) | isobutanol | 3-methy-1-butanol |
|---|---|---|
| 1.5 | 21.33 | 28.99 |
| 3.0 | 16.66 | 21.98 |

According to the calculation above, we assumed that 18-19 mole H2 are needed to form 1 mole isobutanol. Given that the energy densities of H2 and isobutanol are 143 MJ/kg and 36.1 MJ/kg, respectively, the energy efficiency from H2 to isobutanol is 51.9-49.1%. The efficiency of 50% is used.

The disclosure exemplified, in certain embodiment, the isobutanol and 3MB production pathway. The isobutanol and 3MB production pathway converts the keto acid intermediates of amino acid biosynthesis, 2-ketoisovalerate (KIV) and 2-Ketoisocaproate (KIC), into biofuels through two non-native steps borrowed from the Ehrlich pathway: decarboxylation and reduction (FIG. 1B). A multiple-copy plasmid was used to overexpress the keto acid decarboxylase (KDC) kivd along with one of the three different alcohol dehydrogenases (ADH): adhA from Lactococcus lactis, ADH2 from Saccharomyces cerevisiae, and yqhD from Escherichia. coli. Among these ADH's, YqhD is NADPH-dependent, while the others are NADH-dependent. The wildtype Ralstonia eutropha H16 with kivd and yqhD overexpression produced the highest amount of isobutanol from 2-keto isovalerate (KIV) with the lowest amount isobutyraldehyde accumulated (FIG. 2A). This result is consistent with the fact that the highly efficient polyhydroxyalkanoate (PHA) production pathway in this organism uses NADPH as the reducing cofactor, suggesting that there is an abundant NADPH supply in the cell.

These results pinpointed the availability of different reducing cofactors in the cell under heterotrophic growth on fructose. In the lithoautotrophic biofuel production scenario, the oxidation of H2 or formate directly yields NADH. But R. eutropha is equipped with an unusually high number of transhydrogenase isoenzymes that convert NADH to NADPH (FIG. 1A). Indeed, previous studies have shown that NADPH/NADP+ ratio is much higher than that of NADH/NAD+ under autotrophic condition, suggesting that the NADPH dependent aldehyde reduction catalyzed by YqhD may also be favorable for biofuel production from CO2.

Without keto acids added to the medium, biofuel production from fructose by the wildtype strain H16 with kivd and yqhD overexpression reached only ˜1.7 mg/L of isobutanol and ˜3.8 mg/L of 3MB (FIG. 2B). These data suggest the necessity for the enhancement of the native keto acid chain elongation pathway. To do so, the strong phaC1 promoter that drives the expression of the host's PHA synthesis operon (phaC1AB1) was knocked-in in front of the ilvBHC operon and the ilvD gene in R. eutropha H16 genome, which encode the enzymes responsible for the branched chain amino acid biosynthesis (FIG. 2C). The resulting strain LH75 showed significantly higher levels of acetohydroxy-acid synthase (AHAS), IlvC, and IlvD enzyme activities compared to the wildtype when assayed in vitro using cell lysate (FIG. 2E,F,G). Unfortunately, when the kivd and yqhD cassette was introduced to LH75 to form strain LH106, the isobutanol and 3MB productivities on fructose were similar to the wildtype strain H16 transformed with the same Ehrlich cassette but without enhancement of the amino acid pathway (FIG. 2B).

The high enzymatic activity in vitro and low productivity in vivo suggests that post-translational regulations on the native enzymes may control the flux. In fact, the anabolic AHAS enzymes that catalyzed the first-committed step of the keto acid chain elongation are well-known for their strict feedback inhibition by pathway end products and intermediates. To disrupt the post-translational regulation, a catabolic AHAS encoded by alsS from Bacillus subtilis was used (22), which has high specificity to pyruvate and is not subjected to feedback inhibition. The alsS gene together with ilvC and ilvD genes from E. coli were cloned to form a synthetic operon driven by the Ralstonia phaC1 promoter, which was then integrated into the R. eutropha H16 genome to replace the native phaB2C2 operon (FIG. 2D). The resulting strain LH67, although only showing marginally elevated enzymatic activities in vitro (FIG. 2E,F,G) compared to LH75, did provide more keto acid intermediates for biofuel production in vivo: when kivd and yqhD were introduced to LH67, the resulting strain LH74 produced ˜155 mg/L isobutanol and ˜142 mg/L 3MB under the same conditions as described above (FIG. 2B). The isobutanol and 3MB titer was about 30-fold higher than that of LH106 (described above). To integrate the fuel production pathways with host metabolism, the PHB biosynthesis genes phaC1AB1 in strain LH74 were disrupted by a chloramphenicol acetyltransferase (CAT) cassette to give rise to the production strain LH74D (FIG. 3A), which produced isobutanol and 3MB to ˜176 mg/L and ˜160 mg/L from fructose.

After demonstrating its isobutanol and 3MB productivity heterotrophically, LH74D was tested for autotrophic biofuel production on CO₂ and H2. The O₂CO₂ flow rate was adjusted accordingly to keep the ratio of H₂:CO₂:O₂=8:1:1. Under these conditions, the strain LH74D was able to produce a final titer exceeding 1 g/L of fuels (˜536 mg/L isobutanol and ˜520 mg/L 3MB) in 5 days in the J minimal medium (FIG. 3B). Notably, the maximal production rate was reached at ˜380 mg L-1 day-1 and ˜400 mg L-1 day-1 for isobutanol and 3MB, respectively, when the cells entered the stationary phase, indicating high metabolic flux through the engineered biofuel production pathway. This result demonstrates the feasibility of using hydrogen to drive CO₂ reduction to isobutanol and 3MB. However, the low solubility and mass transfer of hydrogen limits the efficiency of its utilization by the cells.

The feasibility of using formic acid as the diffusible and soluble reducing power was then tested. Formic acid, or formate, is toxic to microbial cells at high concentrations because the protonated acid molecules penetrate the cell membrane and acidify the cytoplasm upon proton dissociation. As a result, the proton motive force across the membrane is reduced. To keep a constant low formate concentration in cell culture, pH-coupled formic acid feeding was used to add formic acid in small increments. These conditions enabled normal cell growth and relatively high biofuel productivity (FIG. 3C) in the J minimal medium. The final titer of fuels was over 1.4 g/L (˜846 mg/L isobutanol and ˜570 mg/L 3MB) in around 5 days. Also, the specific productivity of fuels from formate (87.9 mg L-1/day/OD) was much higher than that from hydrogen and CO₂ (9.2 mg L-1/day/OD). Although the peak productivity from formate to fuels (25 mg/L/h) is about 10-fold less than that demonstrated from glucose to isobutanol using E. coli in un-optimized shake flasks, further improvement in productivity can be expected using existing technologies.

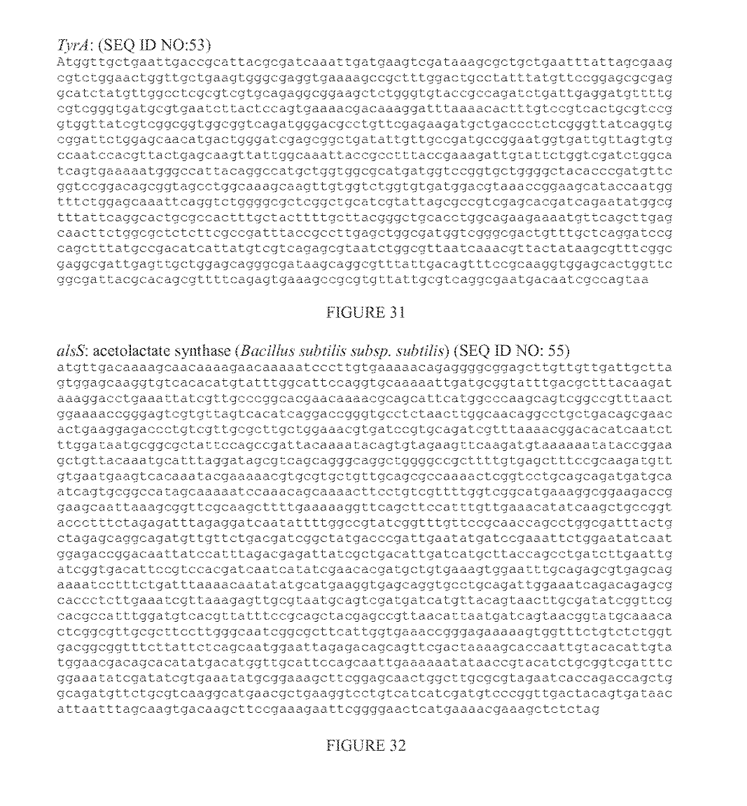

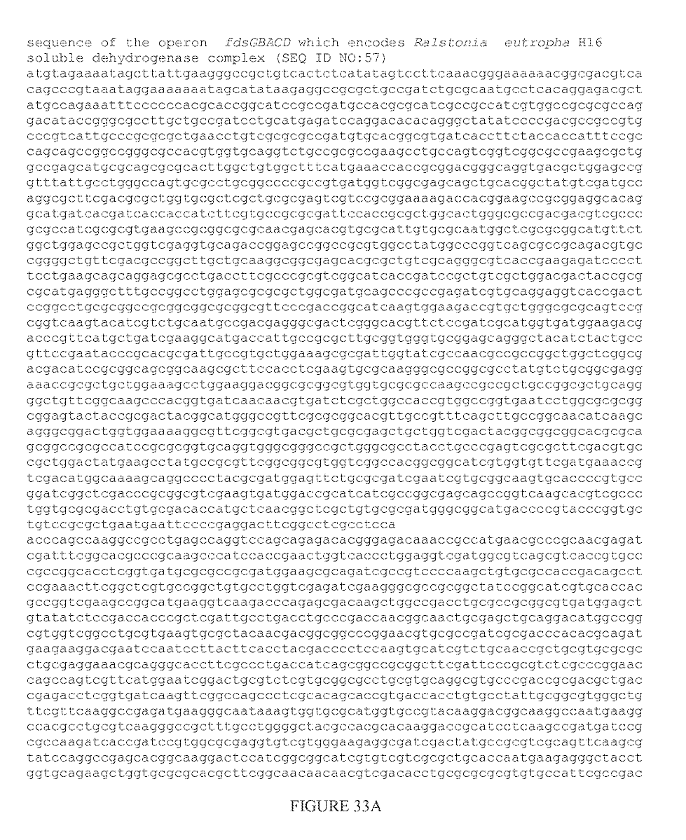

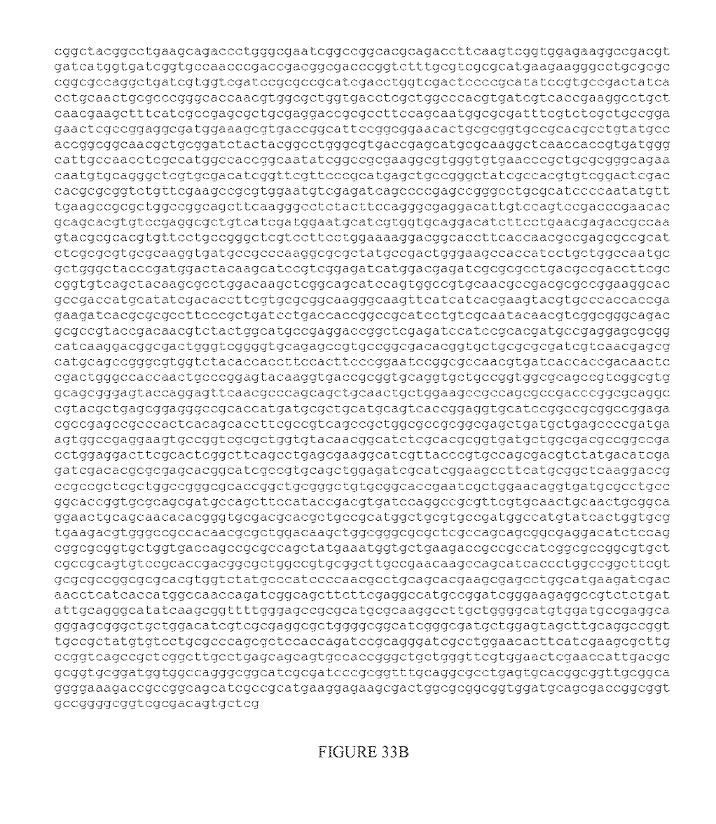

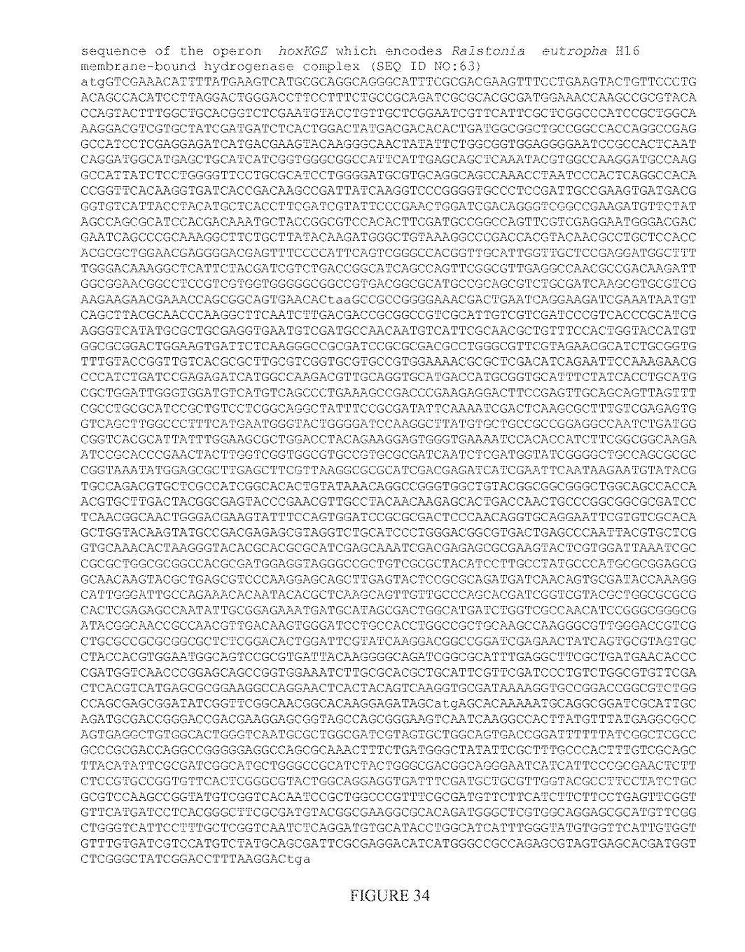

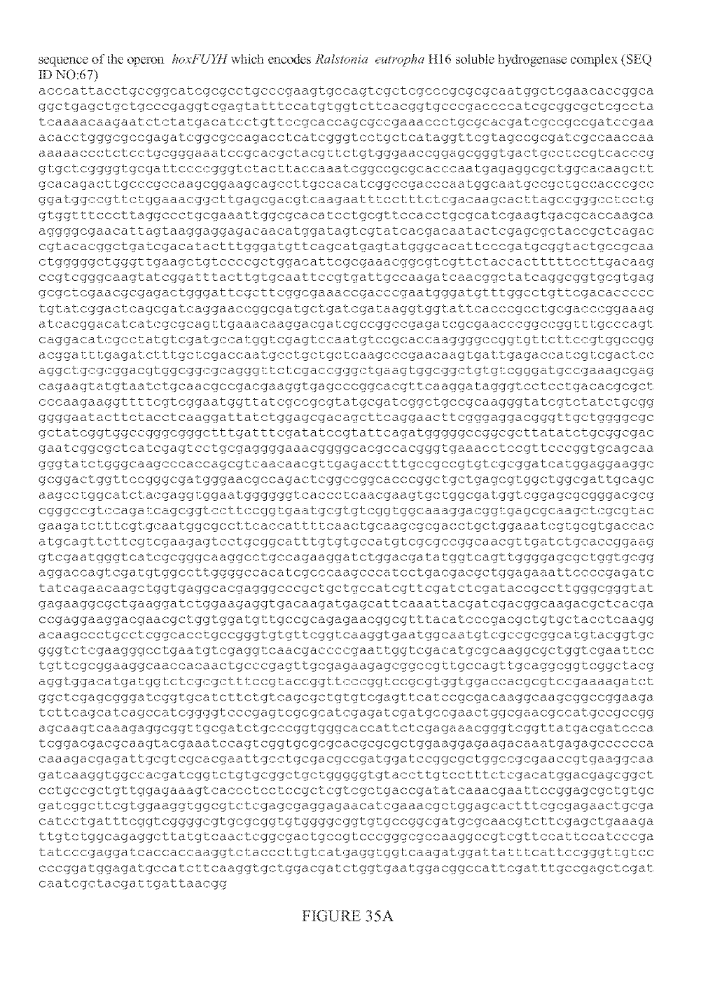

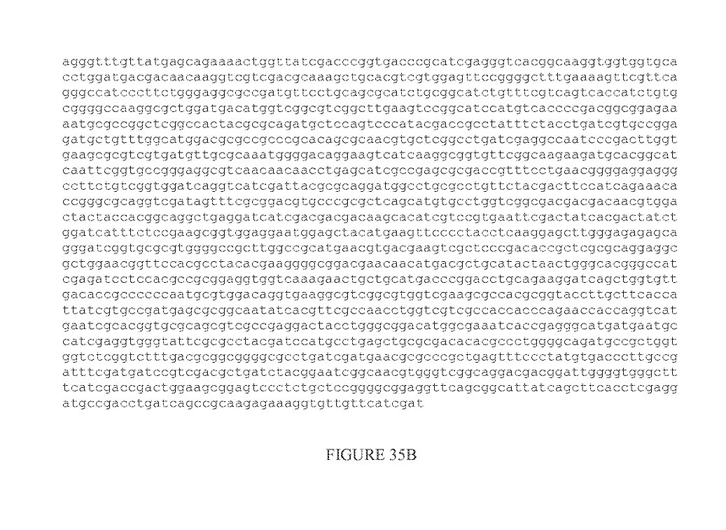

As discussed previously, supplying formate by in-situ electrochemical CO₂ reduction in culture medium may eventually increase efficiency and avoid product purification (FIG. 4A). To test the feasibility of an integrated electro-microbial process, we tested Pb, In, Zn and other metals (10) as a cathode to reduce CO₂ to formic acid with H₂O as the proton source. At the anode (Pt mesh), O₂ is produced from H2O, and is conveniently utilized by Ralstonia in the integrated process. By voltammetry study and the Faradaic yield measurement, we determined that the optimal potential is around −1.6V against the Ag/AgCl reference electrode for the formate production reaction using an In plate cathode in the German minimal medium bubbled with air containing 15% CO₂. Under these conditions, formate can be generated at a relatively high rate, with hydrogen generated as a by-product. Both formate and hydrogen can serve as the energy source to support cell growth and biofuel production (FIGS. 31B, C). Since electrolysis produces fine H₂ bubbles, mass transfer rate can be increased without mechanically dispersing large volume of gas substrate, which is a significant energy cost in the conventional fermentation processes. Thus, hydrogen by-product will not be wasted.

However, when Ralstonia cells were inoculated in the electrochemical reactor, no growth was observed. A growth study using the fast-growing microorganism E. coli showed transient inhibition of electrolysis on cell growth (FIG. 4B). One possibility is the unstable toxic compounds might be produced in the electrolysis reaction. When electricity is turned off, the inhibitory compounds decay quickly and the cell growth is resumed. It was hypothesized that reactive oxygen species and reactive nitrogen species may be generated by the anode, thus causing growth inhibition. To test this hypothesis, three plasmid-based reporter constructs were assembled. Each of the plasmids contain a lacZ gene driven by the promoter of the Ralstonia gene katG (encoding a catalase), sodC (encoding a copper-zinc superoxide dismutase), or norA (encoding an iron-sulfur cluster repair di-iron protein). The promoters of katG, sodC and norA have been shown to be activated by hydrogen peroxide (H₂O₂), superoxide free-radicals (O₂⁻) and nitric oxide (NO), respectively. The plasmids were then transformed into the wild type Ralstonia strain H16. When the plasmid-bearing strains were exposed to electrolysis, expression of β-galactosidase form both sodC and norA promoters where greatly induced, but not for katG promoter (FIG. 4C). These results were consistent with the arguments that O₂⁻ and NO might be generated on the Pt anode and suggested that these unstable reactive compounds trigger stress responses in Ralstonia cells and may be responsible for the transient growth inhibition.

To circumvent this toxicity problem, a porous ceramic cup was used to separate the cathodes and the anode (FIG. 4D). The porous ceramic material provides a tortuous diffusion path for chemicals. Therefore, the reactive compounds produced on the anode inside the cup may be decomposed before reaching the cells growing outside the cup. This strategy is more economical compared to the use of ion-exchange membranes to separate the electrodes. Using this approach healthy growth of Ralstonia biofuel production strain LH74D on electricity and CO₂ was achieved. Over 140 mg/L biofuels were produced in 4 days (FIG. 4E). Further optimization of the culture condition is needed to achieve high productivity over a prolonged time period.

The disclosure demonstrates the feasibility of conversion of electricity to high-energy-density liquid fuels in an integrated process using an engineered R. eutropha strain as the biocatalyst and CO₂ as the carbon source. The electro-microbial process first generates formate or hydrogen as the diffusible reducing intermediates, which then drive the microbial reduction of CO₂ to isobutanol and 3MB. This process does not depend on the biological “light reaction”; and the electricity generated from photovoltaic cells or wind turbines, or off-peak grid power can be used to drive CO₂ fixation and fuel production. Thus, it provides a way to store intermittent renewable energy in liquid transportation fuel with high energy density.

The separation of the “light” and “dark” reactions avoids the simultaneous demand of light exposing surface area and culture containing volume in typical photo-bioreactors. Electricity can be generated and transmitted to remotely power fuel synthesis in the vicinity of a CO₂ source. The use of diffusible reducing intermediates minimizes the dependence on electrode surface area. The use of formate provides further advantages in large scale operations. Upon entering the cell, formate is converted to CO₂ and NADH by formate dehydrogenase, thus providing an inexpensive way to deliver both CO2 and reducing power into the cell. The high solubility of formate and its safety features are highly attractive. Furthermore, since formate is the major byproduct of biomass processing, transformation of formate into liquid fuel compatible with transportation needs using this technology will also play an important role in the biomass refinery process. The approach demonstrated here can also be applicable to produce other chemicals, thus opening the possibility of electricity-driven bioconversion of CO₂ to a variety of chemicals.

To realize the potential of this process, both the electrochemical production of formate and the microbial production of higher alcohols needs to be optimized. The theoretical energy efficiency from H₂ or formate to isobutanol is about 50% (supplementary information). In mature microbial processes, 40-90% of theoretical efficiency can be achieved. If the energy efficiency of electrochemical production of formate or H₂ can be as high as 50-80%, then the overall energy efficiency of electricity to higher alcohols can be 10-36%. Currently, the photovoltaic solar cells commonly achieve 10-20% of energy efficiency. Taken together, the overall solar-to-fuel efficiency by coupling photovoltaic-energy generation to the integrated electro-microbial fuel production can be 1-7.2%. If such efficiencies are achieved, the electro-microbial process compares favorably to the biological photosynthesis-derived fuels or chemicals.

The examples set forth above are provided to give those of ordinary skill in the art a complete disclosure and description of how to make and use the embodiments of the devices, systems and methods of the disclosure, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

A number of embodiments of the invention have been described. Nevertheless, it will be understood that various modifications may be made without departing from the spirit and scope of the invention. Accordingly, other embodiments are within the scope of the following claims.

1. An integrated bioreactor comprising
(a) an anode;
(b) a cathode;
(c) a container comprising at least one wall and having at least one opening, wherein the anode and cathode are disposed within the container;
(d) a liquid permeable separator, wherein the separator surrounds the anode defining an anode space, wherein the separator substantially confines free-radicals produced at the anode within the anode space;
(e) at least one fluid inlet extending through the opening of the container into the container; and
(f) a recombinant microorganism within the container, the recombinant microorganism comprising:
(1) a formate dehydrogenase capable of oxidizing formate and producing NADH or NADPH, or a membrane and/or soluble hydrogenase capable of oxidizing formate and producing NADH or NADPH; and
(2) a heterologous enzyme selected from a ketoacid decarboxylase, an NADPH dependent aldehyde/alcohol dehydrogenase and a combination thereof,
wherein the recombinant microorganism produces an alcohol selected from the group consisting of isobutanol, 1-butanol, 1-propanol, 2-methyl-1-butanol, 3-methyl-1-butanol and 2-phenylethanol from a 2-keto acid intermediate.

2. The integrated bioreactor of claim 1, wherein the at least one fluid inlet comprises at least 2 inlets.

3. The integrated bioreactor of claim 2, wherein the at least one fluid inlet is fluidly connected to a CO₂ sparger.

4. The integrated bioreactor of claim 2, wherein the separator comprises porous ceramic.

5. The integrated bioreactor of claim 1, further comprising an aqueous media suitable for growth of a microorganism.

6. (canceled)

7. The integrated bioreactor of claim 1, wherein the formate dehydrogenase is heterologous.

8. The integrated bioreactor of claim 7, wherein the recombinant microorganism comprises a trans-hydrogenase.

9. The integrated bioreactor of claim 1, wherein the recombinant microorganism is a chemoautotrophic microorganism.

10. The integrated bioreactor of claim 1, wherein the recombinant microorganism is a lithoautotrophic microorganism.

11. (canceled)

12. The integrated bioreactor of claim 1, wherein the membrane and/or soluble hydrogenase is heterologous.

13. The integrated bioreactor of claim 12, wherein the recombinant microorganism comprises a trans-hydrogenase.

14-15. (canceled)

16. A integrated bioreactor of claim 1, wherein the microorganism comprises a carbon fixing enzyme.

17. The integrated bioreactor of claim 16, wherein the carbon fixing enzyme is heterologous to the organism.

18. The integrated bioreactor of claim 1, wherein the biosynthetic pathway for the production of an amino acid in the organism is modified for production of the alcohol.

19. The integrated bioreactor of claim 1, wherein the 2-keto acid intermediate is selected from the group consisting of 2-ketobutyrate, 2-ketoisovalerate, 2-ketovalerate, 2-keto 3-methylvalerate, 2-keto 4-methyl-pentanoate, and phenylpyruvate.

20. The integrated bioreactor of claim 1, wherein the microorganism comprises reduced ethanol production capability compared to a parental microorganism.

21. The integrated bioreactor of claim 20, wherein the microorganism comprises a reduction or inhibition in the conversion of acetyl-coA to ethanol.

22-24. (canceled)

25. The integrated bioreactor of claim 1, wherein the microorganism comprises expression or elevated expression of an enzyme in a biochemical pathway that converts pyruvate to alpha-keto-isovalerate.

26. The integrated bioreactor of claim 1, comprising elevated expression or activity of a 2-keto-acid decarboxylase and an alcohol dehydrogenase, as compared to a parental microorganism.

27. The integrated bioreactor of claim 26, wherein the 2-keto-acid decarboxylase is selected from the group consisting of Pdc, Pdc1, Pdc5, Pdc6, Aro10, Thi3, Kivd, and KdcA, a homolog or variant of any of the foregoing, and a polypeptide having at least 60% identity to any one of the foregoing and having 2-keto-acid decarboxylase activity.

28. (canceled)

29. The integrated bioreactor of claim 1, wherein the alcohol dehydrogenase is selected from the group consisting of Adh1, Adh2, Adh3, Adh4, Adh5, Adh6, Sfa1, a homolog or variant of any of the foregoing, and a polypeptide having at least 60% identity to any one of the foregoing and having alcohol dehydrogenase activity.

30. (canceled)

31. The integrated bioreactor of claim 1, wherein the recombinant microorganism comprises one or more deletions or knockouts in a gene encoding an enzyme that catalyzes the conversion of acetyl-coA to ethanol, catalyzes the conversion of pyruvate to lactate, catalyzes the conversion of fumarate to succinate, catalyzes the conversion of acetyl-coA and phosphate to coA and acetyl phosphate, catalyzes the conversion of acetyl-coA and formate to coA and pyruvate, condensation of the acetyl group of acetyl-CoA with 3-methyl-2-oxobutanoate (2-oxoisovalerate), isomerization between 2-isopropylmalate and 3-isopropylmalate, catalyzes the conversion of alpha-keto acid to branched chain amino acids, synthesis of Phe, Tyr, Asp or Leu, catalyzes the conversion of pyruvate to acetyl-coA, catalyzes the formation of branched chain amino acids, catalyzes the formation of alpha-ketobutyrate from threonine, catalyzes the first step in methionine biosynthesis, catalyzes the conversion of acetoacetyl-CoA to 3-hydroxy-butyryl-Coa, catalyzes the conversion of 3-hydroxy-butyryl-CoA to PHB, and catalyzes the catabolism of threonine.

32. The integrated bioreactor of claim 31, wherein the recombinant microorganism comprises one or more gene deletions selected from the group consisting of adhE, IdhA, frdBC, fnr, pta, pflB, leuA, leuB, leuC, leuD, ilvE, tyrB, poxB, ilvB, ilvI, ivA, metA, tdh, phaA, phaB, phaC, homologs of any of the foregoing and naturally occurring variants of any of the foregoing.

33. The integrated bioreactor of claim 1, comprising a genotype selected from the group consisting of:
(a) a deletion or knockout selected from the group consisting of ΔadhE, ΔldhA, ΔfrdB, ΔfrdC, Δfnr, Δpta, ΔpflB, ΔleuA, ΔilvE, ΔpoxB, ΔilvA, ΔphaA, ΔphaB, ΔphaC and any combination thereof and comprising an expression or increased expression of kdc, ilvC, ilvD and adh2 wherein the microorganism produces isobutanol; and
(b) a deletion or knockout selected from the group consisting of ΔadhE, ΔldhA, ΔfrdB, ΔfrdC, Δfnr, Δpta, ΔpflB, ΔilvE, ΔtyrB, ΔphaA, ΔphaB, ΔphaC and any combination thereof and comprising an expression or increased expression of kdc, LeuABCD, and adh2 wherein the microorganism produces 3-methyl 1-butanol.

34-35. (canceled)

36. The integrated bioreactor of claim 1, wherein the recombinant microorganism has elevated expression or activity of:
a) an acetohydroxy acid synthase;
b) an acetohydroxy acid isomeroreductase;
c) a dihydroxy-acid dehydratase;
d) a 2-keto-acid decarboxylase; and
e) an alcohol dehydrogenase;
as compared to a parental microorganism, and wherein the recombinant microorganism comprises at least one enzyme that can oxidize H₂ or formate to provide free electrons to reduce NAD to NADH or NADP to NADPH, and wherein the organism comprises a carbon fixing pathway that utilizes CO₂ as a carbon source and wherein the organism comprises at least one gene knockout or disruption encoding an enzyme selected from the group consisting of an ethanol dehydrogenase, a lactate dehydrogenase, a fumarate reductase, a phosphate acetyltransferase, a formate acetyltransferase, beta-ketothiolase (phaA), NADPH-linked acetoacetyl coenzyme A (acetyl-CoA) reductase (phaB), and PHB synthase (phaC) and any combination thereof,
wherein the recombinant microorganism produces isobutanol.

37. The integrated bioreactor of claim 1, wherein the recombinant microorganism has elevated expression or activity of:
a) an acetolactate synthase;
b) an acetohydroxy acid isomeroreductase;
c) a dihydroxy-acid dehydratase;
d) a 2-keto-acid decarboxylase; and
e) an alcohol dehydrogenase;
as compared to a parental microorganism, and wherein the recombinant microorganism comprises at least one enzyme that can oxidize H₂ or formate to provide free electrons to reduce NAD to NADH or NADP to NADPH, and wherein the organism comprises a carbon fixing pathway that utilizes CO₂ as a carbon source and wherein the organism comprises at least one gene knockout or disruption encoding an enzyme selected from the group consisting of an ethanol dehydrogenase, a lactate dehydrogenase, a fumarate reductase, a phosphate acetyltransferase, a formate acetyltransferase, beta-ketothiolase (phaA), NADPH-linked acetoacetyl coenzyme A (acetyl-CoA) reductase (phaB), and PHB synthase (phaC) and any combination thereof,
wherein the recombinant microorganism produces isobutanol.

38. The integrated bioreactor of claim 1, wherein the recombinant microorganism has elevated expression or activity of:
a) acetohydroxy acid synthase or acetolactate synthase;
b) acetohydroxy acid isomeroreductase;
c) dihydroxy-acid dehydratase;
d) 2-isopropylmalate synthase;
e) isopropylmalate isomerase
f) beta-isopropylmalate dehydrogenase
g) 2-keto-acid decarboxylase; and
h) alcohol dehydrogenase;
as compared to a parental microorganism, and wherein the recombinant microorganism comprises at least one enzyme that can oxidize H2 or formate to provide free electrons to reduce NAD to NADH or NADP to NADPH, and wherein the organism comprises a carbon fixing pathway that utilizes CO2 as a carbon source and wherein the organism comprises at least one gene knockout or disruption encoding an enzyme selected from the group consisting of an ethanol dehydrogenase, a lactate dehydrogenase, a fumarate reductase, a phosphate acetyltransferase, a formate acetyltransferase, beta-ketothiolase (phaA), NADPH-linked acetoacetyl coenzyme A (acetyl-CoA) reductase (phaB), and PHB synthase (phaC) and any combination thereof.

39. (canceled)

40. The integrated bioreactor of claim 1, wherein the recombinant microorganism is engineered from a parental is Ralstonia sp.

41. The integrated bioreactor of claim 1, wherein the bioreactor produces biofuels from the recombinant microorganism using H₂ or formate for reduction of CO₂, the bioreactor comprising a porous divider that provides a tortuous diffusion path for a growth inhibitor chemical, wherein the divider isolates the anode from a recombinant microorganism.

42. The integrated bioreactor of claim 41, wherein the growth inhibitor chemical is a reactive oxygen species and/or nitric oxide.

43-46. (canceled)
